(19) United States
(12) Patent Application Publication
(10) Pub. No.: US 2024/0254093 A1
(43) Pub. Date: Aug. 1, 2024

(54) GPR35 Agonist Compounds

(71) Applicant: Heptares Therapeutics Limited, Cambridge, Cambridgeshire (GB)

(72) Inventors: Miles Stuart CONGREVE, Cambridgeshire (GB); Nigel Alan SWAIN, Cambridgeshire (GB); Giles Albert BROWN, Cambridgeshire (GB); Benjamin WHITEHURST, Cambridgeshire (GB); Neil John FLANGAN, Cambridgeshire (GB)

(21) Appl. No.: 18/556,178

(22) PCT Filed: Apr. 22, 2022
(86) PCT No.: PCT/IB2022/053772

§ 371 Date: Oct. 19, 2023

(30) Foreign Application Priority Data
Apr. 23, 2021 (GB) 2105846.6

(51) Int. Cl. C07D 257/04; C07D 401/04; C07D 401/10; C07D 403/10

(52) U.S. Cl. CPC C07D 257/04; C07D 401/04; C07D 401/10; C07D 403/10

(57) ABSTRACT

The disclosures herein relate to novel compounds of formula (1):

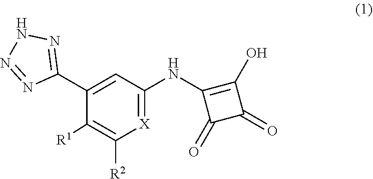

and salts and any tautomers thereof, wherein X, R¹ and R² are defined herein, and their use in treating, preventing, ameliorating, controlling or reducing the risk of disorders associated with GPR35 receptors.

### FIELD OF THE INVENTION

This application relates to compounds and their use as G protein-coupled receptor 35 (GPR35) receptor agonists. Compounds described herein may be useful in the treatment or prevention of diseases in which GPR35 receptors are involved.

### BACKGROUND OF THE INVENTION

GPR35 is an orphan receptor belonging to the family of seven transmembrane domain G protein-coupled receptors (GPCRs). The GPCR superfamily represents a large family of signal transducers which play a key role in regulating various aspects of human physiology. Owing to their pharmacological tractability, these receptors have been intensively studied as potential drug targets. A recent analysis has shown that over 475 drugs act at 108 unique GPCRs, representing ˜34% of FDA approved drugs. There still remains opportunity for the discovery and development of novel drugs for orphan GPCRs for which endogenous ligands have not yet been identified.

GPR35 was originally discovered as an open reading frame encoding a protein of 309 amino acids and localised to human chromosome 2q37.3 (O'Dowd et al. Genomics, 47, 310-3, 1998). The receptor was shown to be expressed in a range of tissues, with high expression reported in gastrointestinal tissues, lung and dorsal root ganglion. The receptor is also found expressed in immune cells, as well as in tissues such as the spleen, skeletal muscle and spinal cord.

Consistent with its expression pattern, there is now accumulating evidence highlighting the therapeutic role for GPR35 across various indications including airway disease, metabolic syndromes (e.g. diabetes), cardiovascular disease (e.g. hypertension) and pain. Ligands that target GPR35 may offer utility for the treatment of a wide range of human disease conditions.

The identity of the endogenous ligand for GPR35 remains a debate and to date a number of putative ligands have been described including 2-acyl lysophosphatidic acid, CXCL17 and kynurenic acid. Many of the reported ligands only show weak activity at the receptor or display lack of biological specificity, raising questions on the true identity of the endogenous ligand.

A wide range of synthetic agonists have been reported to act at GPR35 including zaprinast, pamoic acid, cromolyn, loop diuretic drugs (bumetanide, furosemide), aspirin metabolites, quercetin and dicumarol. In addition, weak agonist activity has also been reported with anti-inflammatory agents sulfasalazine and 5-aminosalicylic acid, which are widely used in the treatment of inflammatory bowel disease (EC50 ˜3 uM) (US20130316985).

Compounds of the tyrosine kinase class, tyrophstin (tyrophstin-51) as well as a catechol-O-methyltransferase (COMT) inhibitor, entacapone have also been reported to act at the receptor highlighting the diversity of the GPR35 ligand class. Owing to the potential involvement of GPR35 in human diseases, there has been an increasing interest in the development of ligands that are highly potent and show selectivity for GPR35. Elucidation of GPR35 biology has somewhat been hampered by the availability of adequate pharmacological tools. Indeed, many of the documented GPR35 agonists (endogenous and synthetic) demonstrate only weak or partial activity at GPR35 and lack target specificity, making the dissection of the pathway difficult. Furthermore, it has become clear that some compounds display species selectivity and possibly ligand bias; the putative endogenous ligand kynurenic acid is one such example where potency at the human receptor is reported to be at least 100 fold lower compared to the rat ortholog (Jenkins et al. Br J Pharmacol 162, 733-748, 2011). Potent, selective GPR35 agonists and antagonists are therefore needed to unravel the physiological role of this receptor.

More recently, genome wide association studies (GWAS) have identified single nucleotide polymorphisms (SNPs) for GPR35 which have been associated with inflammatory bowel disease (IBD). Two SNPs have been described, one of which is a non-synonymous SNP (rs3749171) encoding a T108M substitution in the 3^{rd} transmembrane domain (Ellinghaus et al. Hepatology 58, 1074-1083, 2013). This residue is not conserved throughout mammals and the impact of the polymorphism on signal transduction remains to be elucidated. A second polymorphism at the GPR35 locus (rs4676410) encodes an upstream intron variant of GPR35. A phenome wide association study using 4 large real world data cohorts demonstrated the association of this genetic variant (rs4676410) with an IBD phenotype confirming the target-disease links reported previously in earlier studies (Diogo et al. Nat Commun 9, 4285, 2018). The reported association of GPR35 polymorphism in IBD has raised interest in the potential therapeutic role of GPR35 for the treatment of gastrointestinal diseases.

Inflammatory bowel disease is a chronic relapsing inflammatory gastrointestinal disorder that commonly involves the ileum and/or colon. The pathophysiology is thought to involve an abnormal intestinal immune response, resulting in mucosal inflammation, defective intestinal barrier and increased GI permeability. Treatment strategy largely involves a stepwise approach through the combined use of agents such as aminosalicylates, corticosteroids, immunosuppressants, biologics (e.g. anti-TNF) and antibiotics, however many patients experience incomplete disease control, highlighting the high unmet need. In particular, one aspect which remains poorly treated and underrecognized is abdominal pain. Pain is a common symptom experienced by the vast majority of IBD patients during the disease course and can arise from a direct or indirect consequence of intestinal inflammation. Whatever the cause, pain negatively impacts the quality of life of IBD patients. To date, the management of IBD associated abdominal pain remains challenging. Commonly used analgesics such as non-steroidal anti-inflammatory agents (NSAIDs) can often exacerbate the condition, leaving patients with limited treatment options. There is therefore a high unmet need for agents that can provide fast onset pain relief and development of novel drugs is urgently needed.

In preclinical studies, GPR35 mutant mice display greater degrees of colonic epithelial damage following chemical injury, compared to wildtype mice. GPR35 knockout mice display elevated expression of inflammatory and remodelling cytokines, although numbers of inflammatory cell influx in the mucosa, show no overall difference (Farooq et al. Digestive Diseases and Sciences, 63, 2910-22, 2018). A role of GPR35 in barrier homeostasis has also been reported, with agents such as sodium cromoglycate demonstrating the ability to reduce GI permeability in a number of gut sensitisation models (Forbes et al. J Exp Med 205: 897-913, 2008; Yokooji et al. Int Arch Allergy Immunol. 167:193-202, 2015). Consistent with these findings, GPR35 has been shown to play a role in the regulation of tight junction proteins and promoting epithelial cell migration in in vitro studies. Finally, GPR35 is richly expressed in dorsal root ganglion (DRG) neurones where it has been shown to colocalise with nociceptive ion channels and play a role in pain processing (Ohshiro et al. Biochem. Biophys. Res. Commun. 365, 344-8, 2008). In addition to the reported effects on barrier protection, cromolyn reduces visceral hypersensitivity in a stress sensitive rat strain (Carroll et al. PLoS One.8:e84718, 2013), highlighting its potential utility in the treatment of pain.

Sodium cromoglycate is a mast cell stabiliser approved for a range of indications including systemic mastocytosis, prophylaxis of allergic rhinitis and asthma, allergic conjunctivitis and food allergy (in conjunction with dietary restriction). Use of cromoglycate in systemic mastocytosis is reported to result in improvement of diarrhoea, flushing, headaches, vomiting, urticaria and abdominal pain. Trials evaluating the effectiveness of sodium cromoglicate in food allergy have reported mixed results, with high doses generally required to offer protection. Doses of up to 2 g/day were shown to be effective in attenuating the severity of GI symptoms in patients with irritable bowel syndrome due to food allergy (Lunardi et al. Clin Exp Allergy. 21:569-72, 1991). Similar findings have been reported in children with milk allergy on gastrointestinal permeability endpoint.

In addition to gastrointestinal disorders, GPR35 has received interest as a target for the treatment of allergic disorders including asthma. In the lung, cromolyn has long been used as an effective asthma therapy with good safety and tolerability profile, but with suboptimal pharmacokinetics. It is estimated that approximately 5-12% of the drug is absorbed following deposition in the airways and more recently, an improved formulation of cromolyn has been developed (PA101) which achieves significantly higher drug deposition in the lung.

Clinically, cromolyn shows efficacy in suppressing the immediate and late onset asthmatic response following allergen challenge. In a phase II proof of concept trial, PA101 demonstrated efficacy in reducing the cough frequency in patients with idiopathic pulmonary fibrosis. GPR35 mRNA is upregulated in response to challenge with IgE antibodies and cromolyn has been reported to block inflammatory mediator release in human lung slices passively sensitised with IgE antibodies. These effects are likely to involve a number of mechanisms including regulation of mast cell stabilisation and reflex induced bronchoconstriction.

The emerging data therefore highlights a broad therapeutic potential for GPR35 agonists, ranging from mast cell disorders, treatment of acute and chronic pain conditions and diseases associated with allergic or inflammatory diseases in both the gastrointestinal system and the lung.

### BRIEF SUMMARY OF THE INVENTION

The present invention relates to compounds having activity as G protein-coupled receptor 35 (GPR35) receptor agonists.

Briefly, in one aspect, the invention provides compounds of the formula (1):

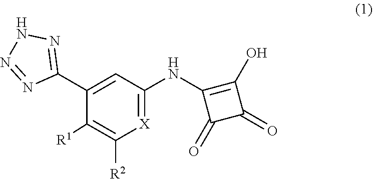

or a salt or tautomer thereof, wherein;
X is N or CH;
R¹ is H or halo;
R² is H, halo, optionally substituted C₁₋₆ alkyl, optionally substituted C₃₋₆ cycloalkyl, optionally substituted C₁₋₆ alkoxy, optionally substituted aryl, optionally substituted heteroaryl or optionally substituted O-aryl.

The compounds may be used as GPR35 receptor agonists. The compounds may be used in the manufacture of medicaments. The compounds may be for use in treating, preventing, ameliorating, controlling or reducing the risk of disorders associated with GPR35. The compounds may be used in the treatment of mast cell disorders, acute and chronic pain conditions and diseases associated with allergic or inflammatory diseases in both the gastrointestinal system and the lung.

### BRIEF DESCRIPTION OF THE FIGURES

FIG. 1 is an X-Ray Powder Diffraction pattern of a crystalline form of Compound A tromethamine salt (Hydrate I).

FIG. 2 is a Differential Scanning Calorimetry curve of a crystalline form of Compound A tromethamine salt (Hydrate I).

FIG. 3 is a Thermal Gravimetric Analysis curve of a crystalline form of Compound A tromethamine salt (Hydrate I).

FIG. 4 is an X-Ray Powder Diffraction pattern of a crystalline form of the tromethamine salt (Hydrate II).

FIG. 5 is a Differential Scanning Calorimetry curve of a crystalline form of Compound A tromethamine salt (Hydrate II).

FIG. 6 is a Thermal Gravimetric Analysis curve of a crystalline form of Compound A tromethamine salt (Hydrate II).

FIG. 7 is an X-Ray Powder Diffraction pattern of a crystalline form of Compound A free acid (Pattern 3).

FIG. 8 is a Thermal Gravimetric Analysis curve of a crystalline form of Compound A free acid (Pattern 3).

FIG. 9 is an X-Ray Powder Diffraction pattern of a crystalline form of Compound A free acid (Pattern 1).

FIG. 10 is a Thermal Gravimetric Analysis curve of a crystalline form of Compound A free acid (Pattern 1).

### DETAILED DESCRIPTION OF THE INVENTION

The invention relates to novel compounds. The invention relates to the use of novel compounds as agonists of the GPR35 receptor. The invention also relates to the use of novel compounds in the treatment or prevention of diseases in which GPR35 receptors are involved. The invention further relates to the use of novel compounds in the manufacture of medicaments for use as GPR35 receptor agonists.

The invention provides compounds of the formula (1):

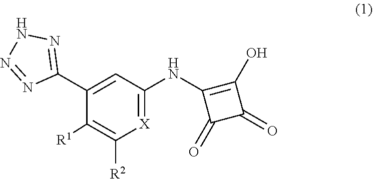

or a salt or tautomer thereof, wherein;
X is N or CH;
R¹ is H or halo;
R² is H, halo, optionally substituted C₁₋₆ alkyl, optionally substituted C₃₋₆ cycloalkyl, optionally substituted C₁₋₆ alkoxy, optionally substituted aryl, optionally substituted heteroaryl or optionally substituted O-aryl.

In the compounds herein, X can be N. X can be CH.

In the compounds herein, R¹ can be H. R¹ can be halo. R¹ can be Cl or F. R¹ can be Cl. R¹ can be F. R¹ can be Br.

In the compounds herein, R² can be H. R² can be halo. R² can be optionally substituted C₁₋₆ alkyl. R² can be optionally substituted C₃₋₆ cycloalkyl. R² can be optionally substituted C₁₋₆ alkoxy. R² can be optionally substituted aryl. R² can be optionally substituted heteroaryl. R² can be optionally substituted monocyclic heteroaryl. R² can be optionally substituted bicyclic heteroaryl. R² can be optionally substituted O-aryl.

R² can be H, C₁₋₆ alkyl optionally substituted with 1 to 6 fluorine atoms, C₃₋₆ cycloalkyl optionally substituted with 1 to 6 fluorine atoms or C₁₋₆ alkoxy optionally substituted with 1 to 6 fluorine atoms. R² can be H, trifluoromethyl, ethyl, cyclopropyl, cyclohexyl or methoxy.

R² can be phenyl optionally substituted with R³, pyridyl optionally substituted with R³, 0-phenyl optionally substituted with R³, indazolyl optionally substituted with R³ or pyridazinyl optionally substituted with R³, wherein R³ is H, halo, C₁₋₆ alkyl optionally substituted with 1 to 6 fluorine atoms, C₃₋₆ cycloalkyl optionally substituted with 1 to 6 fluorine atoms, C₁₋₆ alkoxy optionally substituted with 1 to 6 fluorine atoms, —CO₂R⁴, —CONHCH₂R⁴, —CONHCH₂CH₂OR⁴, —OR⁴, —OCH₂R⁴, —CH₂R⁴, —OCH₂R⁴, —CH₂CH₂OR⁴, —OCH₂CH₂OR⁴, —CONHR⁴ or —CON(CH₃)R⁴; where R⁴ is H, C₁₋₆ alkyl optionally substituted with 1 to 6 fluorine atoms, or a group:

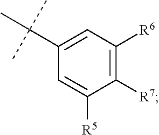

R⁵, R⁶ and R⁷ are independently H, halo, CO₂R⁸, CONR⁸R⁹ or C₁₋₆ alkyl optionally substituted with 1 to 6 fluorine atoms, wherein one or two carbon atoms of the C₁₋₆ alkyl group may be optionally replaced by O;
R⁸ and R⁹ are independently H or C₁₋₆ alkyl optionally substituted with 1 to 6 fluorine atoms.
R² can be optionally substituted aryl, optionally substituted heteroaryl or optionally substituted O-aryl, wherein the optional substituent is R³.
R² can be optionally substituted aryl, optionally substituted O-aryl, optionally substituted heteroaryl, optionally substituted monocyclic heteroaryl or optionally substituted bicyclic heteroaryl, wherein the optional substituent is R³.
R² can be phenyl optionally substituted with R³, pyridyl optionally substituted with R³, O-phenyl optionally substituted with R³, indazolyl optionally substituted with R³ or pyridazinyl optionally substituted with R³.
R³ can be H, halo, C₁₋₆ alkyl optionally substituted with 1 to 6 fluorine atoms, C₃₋₆ cycloalkyl optionally substituted with 1 to 6 fluorine atoms, C₁₋₆ alkoxy optionally substituted with 1 to 6 fluorine atoms, —CO₂R⁴, —CONHCH₂R⁴, —CONHCH₂CH₂OR⁴, —OR⁴, —OCH₂R⁴, —CH₂R⁴, —OCH₂R⁴, —CH₂CH₂OR⁴, —OCH₂CH₂OR⁴, —CONHR⁴ or —CON(CH₃)R⁴;
where R⁴ is H, C₁₋₆ alkyl optionally substituted with 1 to 6 fluorine atoms, or a group:

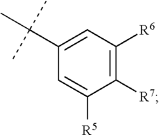

R⁵, R⁶ and R⁷ are independently H, halo, CO₂R⁸, CONR⁸R⁹ or C₁₋₆ alkyl optionally substituted with 1 to 6 fluorine atoms, wherein one or two carbon atoms of the C₁₋₆ alkyl group may be optionally replaced by O;
R⁸ and R⁹ are independently H or C₁₋₆ alkyl optionally substituted with 1 to 6 fluorine atoms.

In the compounds herein, R⁴ can be H, methyl, or selected from the group consisting of:

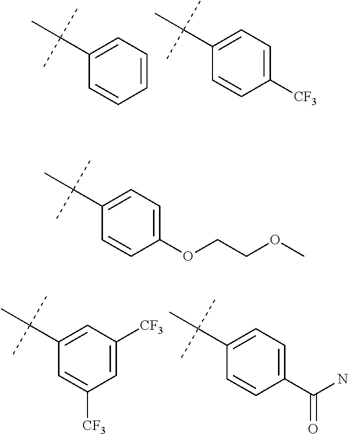

In the compounds herein, R⁵, R⁶ and R⁷ can independently be H, CF₃, CONH₂ or —OCH₂CH₂OCH₃.

In the compounds herein, R⁸ and R⁹ can independently be H or methyl. R³ can be OMe, CO₂H, CO₂Et, CON(CH₃)₂, CONHCH₂CH₂OCH₃, or selected from the group consisting of:

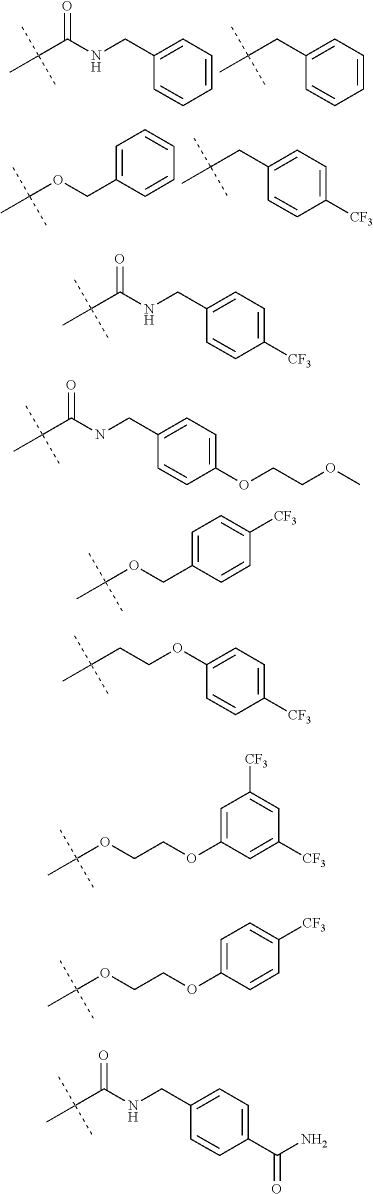

In the compounds herein R² can be selected from the group consisting of:

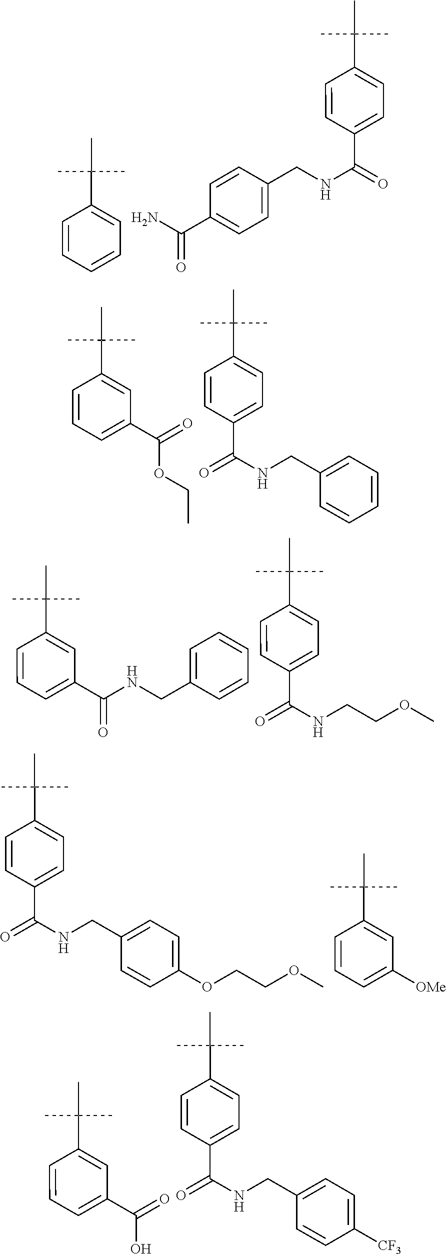

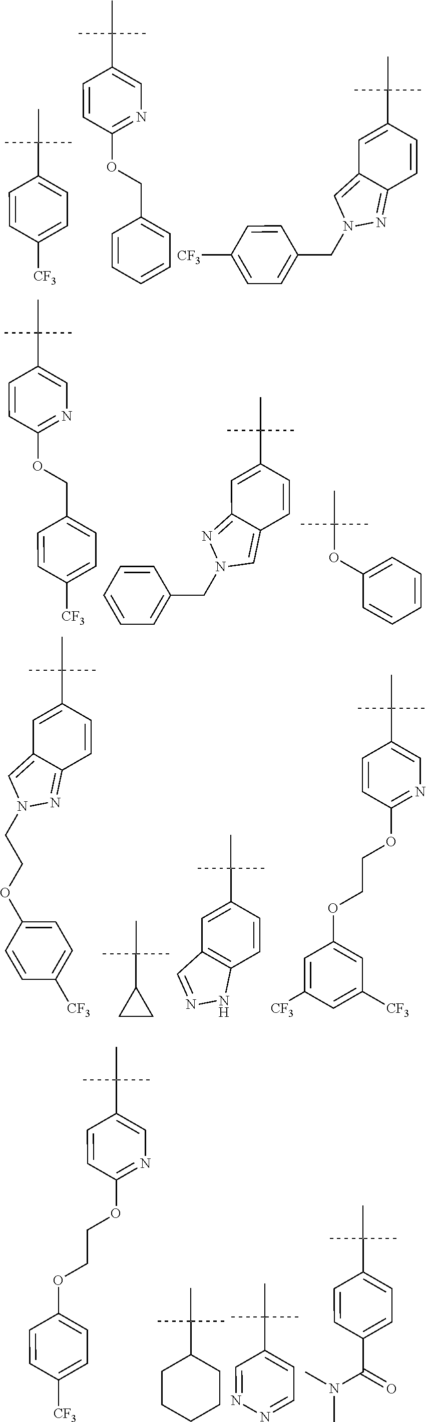

The compound can be a compound of formula (1a) or (1b):

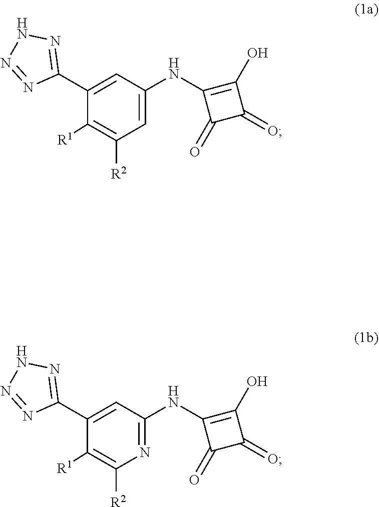

or a salt or tautomer thereof, wherein R¹ and R² are as defined above.

In some embodiments, the compound can be a compound of formula (1a):

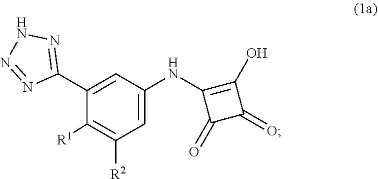

or a salt or tautomer thereof, wherein R¹ and R² are as defined above.

The compound can be a compound of formula (2a) or (2b):

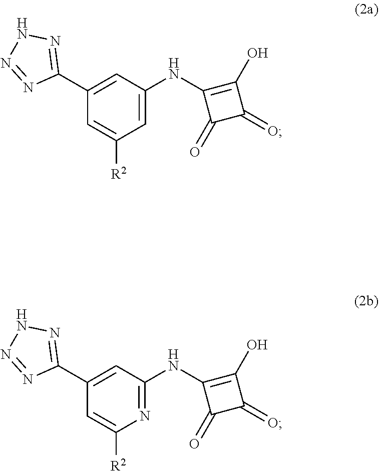

or a salt or tautomer thereof, wherein R² is as defined above.

In some embodiments, the compound can be a compound of formula (2a):

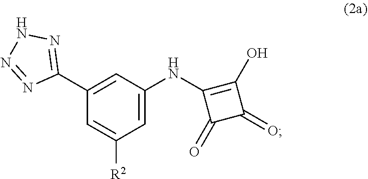

or a salt or tautomer thereof, wherein R² is as defined above.

The compound can be a compound of formula (3a), (3b), (3c), (3d) or (3e):

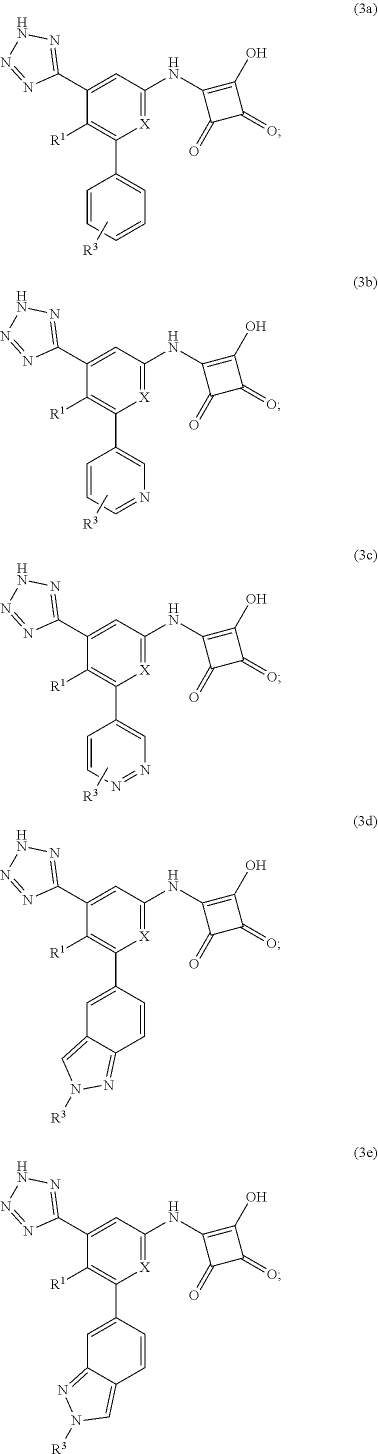

or a salt or tautomer thereof; wherein X, R¹ and R³ are as defined above.

In some embodiments, the compound can be a compound of formula (3a):

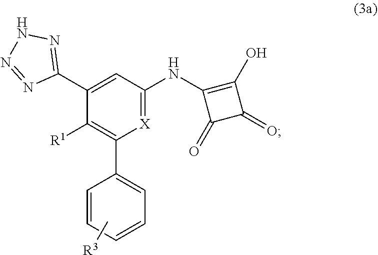

or a salt or tautomer thereof; wherein X, R¹ and R³ are as defined above.

The compound can be a compound of formula (4a), (4b), (4c), (4d) or (4e):

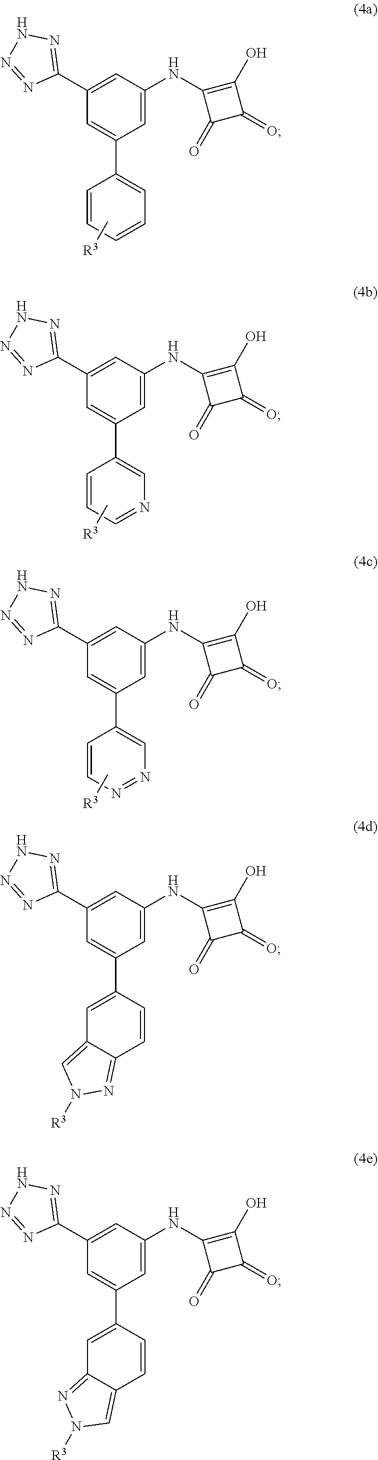

or a salt or tautomer thereof; wherein R³ is as defined above.

In some embodiments, the compound can be a compound of formula (4a):

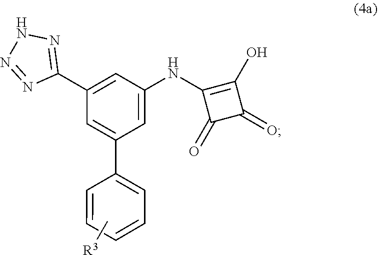

or a salt or tautomer thereof; wherein R³ is as defined above.

The compound can be selected from the group consisting of:

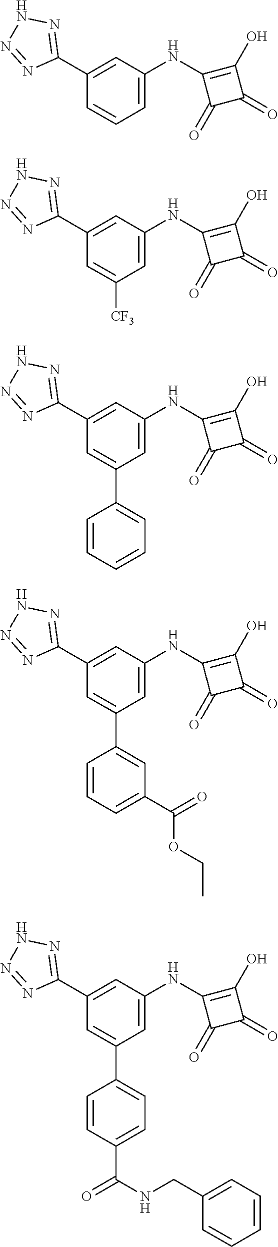 [SMILES: O=c1c(O)c(Nc2cccc(-c3nn[nH]n3)c2)c1=O ; O=c1c(O)c(Nc2cc(-c3nn[nH]n3)cc(C(F)(F)F)c2)c1=O ; O=c1c(O)c(Nc2cc(-c3ccccc3)cc(-c3nn[nH]n3)c2)c1=O ; CCOC(=O)c1cccc(-c2cc(Nc3c(O)c(=O)c3=O)cc(-c3nn[nH]n3)c2)c1 ; O=C(NCc1ccccc1)c1ccc(-c2cc(Nc3c(O)c(=O)c3=O)cc(-c3nn[nH]n3)c2)cc1]

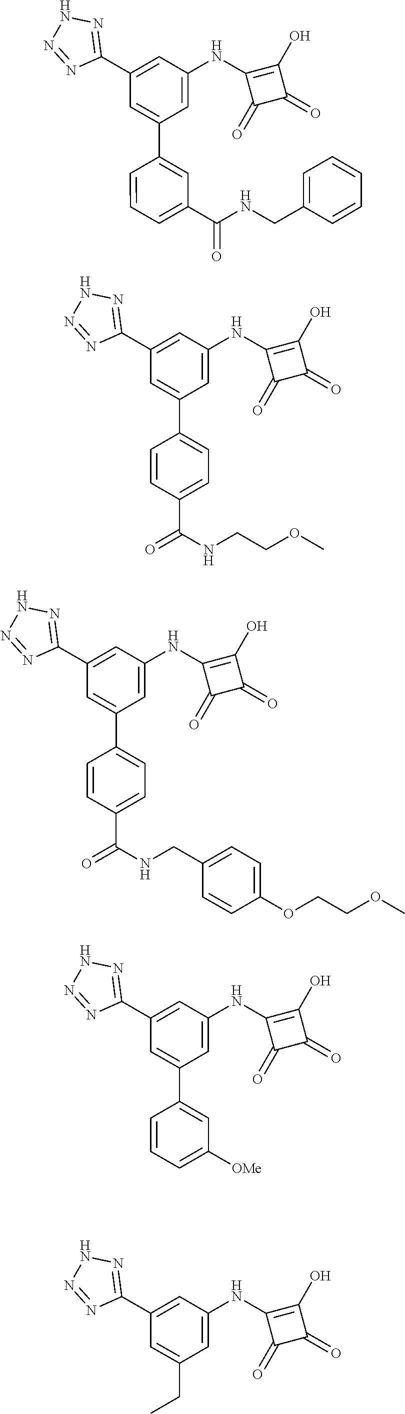 [SMILES: O=C(NCc1ccccc1)c1cccc(-c2cc(Nc3c(O)c(=O)c3=O)cc(-c3nn[nH]n3)c2)c1 ; COCCNC(=O)c1ccc(-c2cc(Nc3c(O)c(=O)c3=O)cc(-c3nn[nH]n3)c2)cc1 ; COCCOc1ccc(CNC(=O)c2ccc(-c3cc(Nc4c(O)c(=O)c4=O)cc(-c4nn[nH]n4)c3)cc2)cc1 ; COc1cccc(-c2cc(Nc3c(O)c(=O)c3=O)cc(-c3nn[nH]n3)c2)c1 ; CCc1cc(Nc2c(O)c(=O)c2=O)cc(-c2nn[nH]n2)c1]

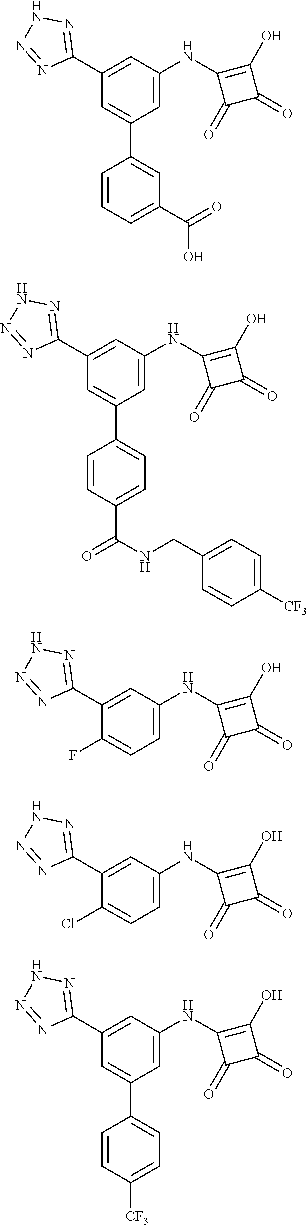 [SMILES: O=C(O)c1cccc(-c2cc(Nc3c(O)c(=O)c3=O)cc(-c3nn[nH]n3)c2)c1 ; O=C(NCc1ccc(C(F)(F)F)cc1)c1ccc(-c2cc(Nc3c(O)c(=O)c3=O)cc(-c3nn[nH]n3)c2)cc1 ; O=c1c(O)c(Nc2ccc(F)c(-c3nn[nH]n3)c2)c1=O ; O=c1c(O)c(Nc2ccc(Cl)c(-c3nn[nH]n3)c2)c1=O ; O=c1c(O)c(Nc2cc(-c3ccc(C(F)(F)F)cc3)cc(-c3nn[nH]n3)c2)c1=O]

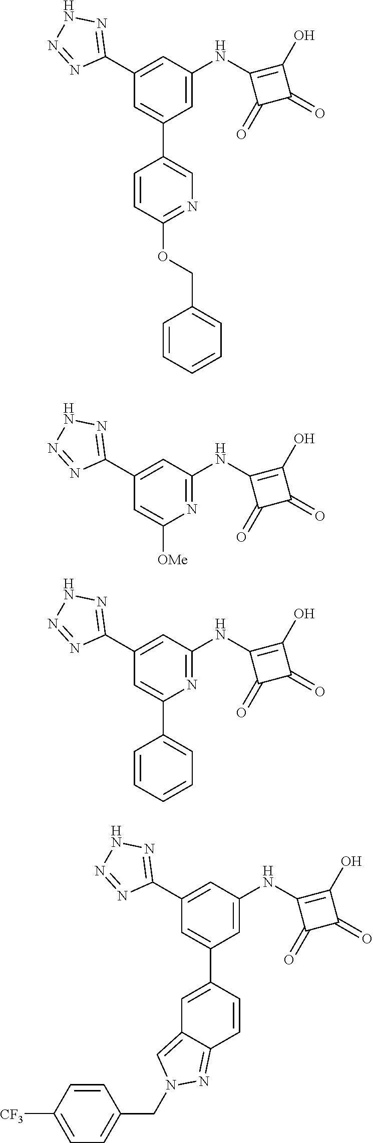 [SMILES: O=c1c(O)c(Nc2cc(-c3ccc(OCc4ccccc4)nc3)cc(-c3nn[nH]n3)c2)c1=O ; COc1cc(-c2nn[nH]n2)cc(Nc2c(O)c(=O)c2=O)n1 ; O=c1c(O)c(Nc2cc(-c3nn[nH]n3)cc(-c3ccccc3)n2)c1=O ; O=c1c(O)c(Nc2cc(-c3ccc4nn(Cc5ccc(C(F)(F)F)cc5)cc4c3)cc(-c3nn[nH]n3)c2)c1=O]

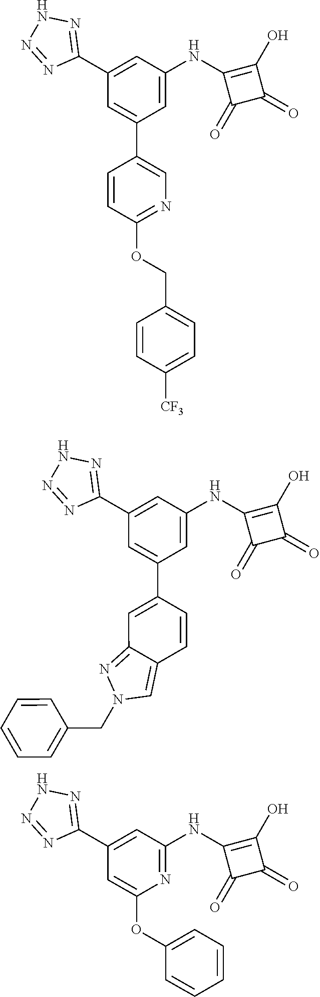

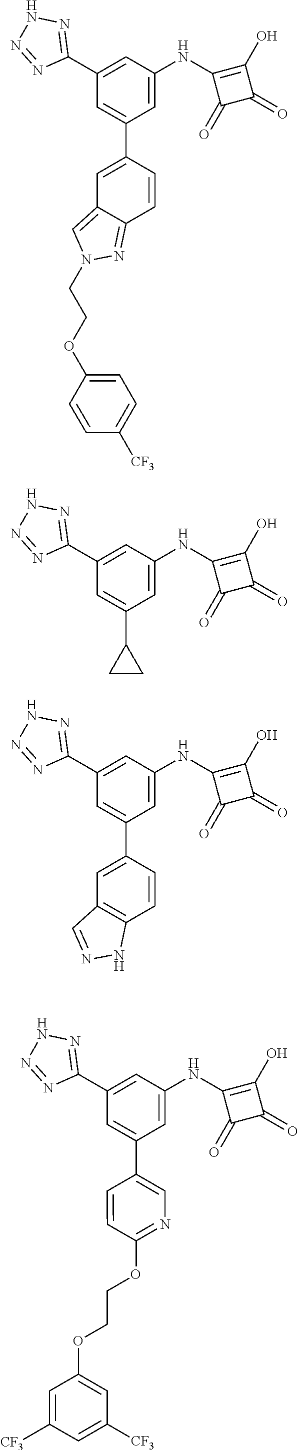 [SMILES: O=c1c(O)c(Nc2cc(-c3ccc4nn(CCOc5ccc(C(F)(F)F)cc5)cc4c3)cc(-c3nn[nH]n3)c2)c1=O ; O=c1c(O)c(Nc2cc(-c3nn[nH]n3)cc(C3CC3)c2)c1=O ; O=c1c(O)c(Nc2cc(-c3ccc4[nH]ncc4c3)cc(-c3nn[nH]n3)c2)c1=O ; O=c1c(O)c(Nc2cc(-c3ccc(OCCOc4cc(C(F)(F)F)cc(C(F)(F)F)c4)nc3)cc(-c3nn[nH]n3)c2)c1=O]

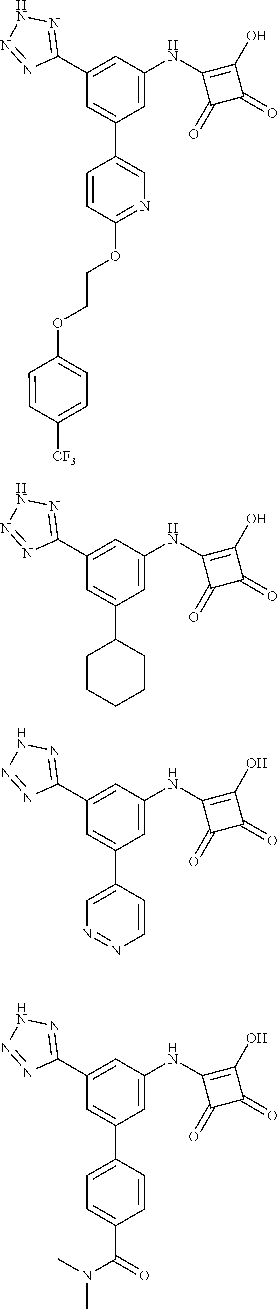 [SMILES: O=c1c(O)c(Nc2cc(-c3ccc(OCCOc4ccc(C(F)(F)F)cc4)nc3)cc(-c3nn[nH]n3)c2)c1=O ; O=c1c(O)c(Nc2cc(-c3nn[nH]n3)cc(C3CCCCC3)c2)c1=O ; O=c1c(O)c(Nc2cc(-c3ccnnc3)cc(-c3nn[nH]n3)c2)c1=O ; CN(C)C(=O)c1ccc(-c2cc(Nc3c(O)c(=O)c3=O)cc(-c3nn[nH]n3)c2)cc1]

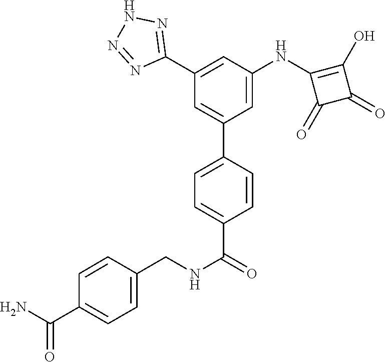 [SMILES: NC(=O)c1ccc(CNC(=O)c2ccc(-c3cc(Nc4c(O)c(=O)c4=O)cc(-c4nn[nH]n4)c3)cc2)cc1]

or a salt or tautomer thereof.

The compound can be selected from the group consisting of:
3-((3-(1H-tetrazol-5-yl)phenyl)amino)-4-hydroxycyclobut-3-ene-1,2-dione;
3-((3-(1H-tetrazol-5-yl)-5-(trifluoromethyl)phenyl)amino)-4-hydroxycyclobut-3-ene-1,2-dione;
3-((5-(1H-tetrazol-5-yl)-[1,1′-biphenyl]-3-yl)amino)-4-hydroxycyclobut-3-ene-1,2-dione;
ethyl 3′-((2-hydroxy-3,4-dioxocyclobut-1-en-1-yl)amino)-5′-(1H-tetrazol-5-yl)-[1,1′-biphenyl]-3-carboxylate;
N-benzyl-3′-((2-hydroxy-3,4-dioxocyclobut-1-en-1-yl)amino)-5′-(1H-tetrazol-5-yl)-[1,1′-biphenyl]-4-carboxamide;
N-benzyl-3′-((2-hydroxy-3,4-dioxocyclobut-1-en-1-yl)amino)-5′-(1H-tetrazol-5-yl)-[1,1′-biphenyl]-3-carboxamide;
3′-((2-hydroxy-3,4-dioxocyclobut-1-en-1-yl)amino)-N-(2-methoxyethyl)-5′-(1H-tetrazol-5-yl)-[1,1′-biphenyl]-4-carboxamide;
3′-((2-hydroxy-3,4-dioxocyclobut-1-en-1-yl)amino)-N-(4-(2-methoxyethoxy)benzyl)-5′-(1H-tetrazol-5-yl)-[1,1′-biphenyl]-4-carboxamide;
3-hydroxy-4-((3′-methoxy-5-(1H-tetrazol-5-yl)-[1,1′-biphenyl]-3-yl)amino)cyclobut-3-ene-1,2-dione;
3-((3-ethyl-5-(1H-tetrazol-5-yl)phenyl)amino)-4-hydroxycyclobut-3-ene-1,2-dione;
3′-((2-hydroxy-3,4-dioxocyclobut-1-en-1-yl)amino)-5′-(1H-tetrazol-5-yl)-[1,1′-biphenyl]-3-carboxylic acid;
3′-((2-hydroxy-3,4-dioxocyclobut-1-en-1-yl)amino)-5′-(1H-tetrazol-5-yl)-N-(4-(trifluoromethyl)benzyl)-[1,1′-biphenyl]-4-carboxamide;
3-((4-fluoro-3-(1H-tetrazol-5-yl)phenyl)amino)-4-hydroxycyclobut-3-ene-1,2-dione;
3-((4-chloro-3-(1H-tetrazol-5-yl)phenyl)amino)-4-hydroxycyclobut-3-ene-1,2-dione;
3-((5-(1H-tetrazol-5-yl)-4′-(trifluoromethyl)-[1,1′-biphenyl]-3-yl)amino)-4-hydroxycyclobut-3-ene-1,2-dione;
3-((3-(6-(benzyloxy)pyridin-3-yl)-5-(1H-tetrazol-5-yl)phenyl)amino)-4-hydroxycyclobut-3-ene-1,2-dione;
3-hydroxy-4-((6-methoxy-4-(1H-tetrazol-5-yl)pyridin-2-yl)amino)cyclobut-3-ene-1,2-dione;
3-hydroxy-4-((6-phenyl-4-(1H-tetrazol-5-yl)pyridin-2-yl)amino)cyclobut-3-ene-1,2-dione;
3-((3-(1H-tetrazol-5-yl)-5-(2-(4-(trifluoromethyl)benzyl)-2H-indazol-5-yl)phenyl)amino)-4-hydroxycyclobut-3-ene-1,2-dione;
3-((3-(1H-tetrazol-5-yl)-5-(6-((4-(trifluoromethyl)benzyl)oxy)pyridin-3-yl)phenyl)amino)-4-hydroxycyclobut-3-ene-1,2-dione;
3-((3-(2-benzyl-2H-indazol-5-yl)-5-(1H-tetrazol-5-yl)phenyl)amino)-4-hydroxycyclobut-3-ene-1,2-dione;
3-hydroxy-4-((6-phenoxy-4-(1H-tetrazol-5-yl)pyridin-2-yl)amino)cyclobut-3-ene-1,2-dione;
3-((3-(1H-tetrazol-5-yl)-5-(2-(2-(4-(trifluoromethyl)phenoxy)ethyl)-2H-indazol-6-yl)phenyl)amino)-4-hydroxycyclobut-3-ene-1,2-dione;
3-((3-cyclopropyl-5-(1H-tetrazol-5-yl)phenyl)amino)-4-hydroxycyclobut-3-ene-1,2-dione;
3-((3-(1H-indazol-5-yl)-5-(1H-tetrazol-5-yl)phenyl)amino)-4-hydroxycyclobut-3-ene-1,2-dione;
3-((3-(6-(2-(3,5-bis(trifluoromethyl)phenoxy)ethoxy)pyridin-3-yl)-5-(1H-tetrazol-5-yl)phenyl)amino)-4-hydroxycyclobut-3-ene-1,2-dione;
3-((3-(1H-tetrazol-5-yl)-5-(6-(2-(4-(trifluoromethyl)phenoxy)ethoxy)pyridin-3-yl)phenyl)amino)-4-hydroxycyclobut-3-ene-1,2-dione;
3-((3-cyclohexyl-5-(1H-tetrazol-5-yl)phenyl)amino)-4-hydroxycyclobut-3-ene-1,2-dione;
3-hydroxy-4-((3-(pyridazin-4-yl)-5-(1H-tetrazol-5-yl)phenyl)amino)cyclobut-3-ene-1,2-dione;
3′-((2-hydroxy-3,4-dioxocyclobut-1-en-1-yl)amino)-N,N-dimethyl-5′-(1H-tetrazol-5-yl)-[1,1′-biphenyl]-4-carboxamide;
N-(4-carbamoylbenzyl)-3′-((2-hydroxy-3,4-dioxocyclobut-1-en-1-yl)amino)-5′-(1H-tetrazol-5-yl)-[1,1′-biphenyl]-4-carboxamide;
   or a salt or tautomer thereof.

In some embodiments, the salt of the compound of the formula (1) is a pharmaceutically acceptable salt.

In some embodiments, the compound is a compound having the structure:

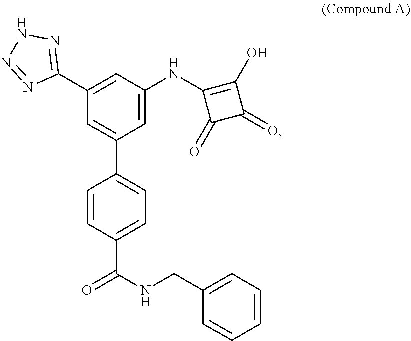 [SMILES: O=C(NCc1ccccc1)c1ccc(-c2cc(Nc3c(O)c(=O)c3=O)cc(-c3nn[nH]n3)c2)cc1]

or a pharmaceutically acceptable salt thereof. In some embodiments, the compound is a compound having the structure:

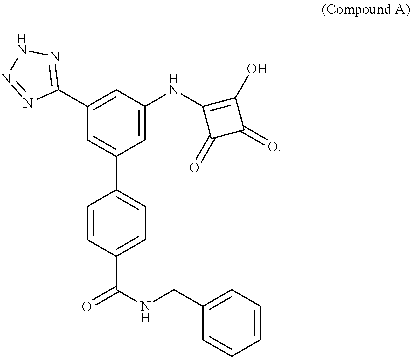 [SMILES: O=C(NCc1ccccc1)c1ccc(-c2cc(Nc3c(O)c(=O)c3=O)cc(-c3nn[nH]n3)c2)cc1]

The present disclosure also provides a tromethamine salt having the structure:

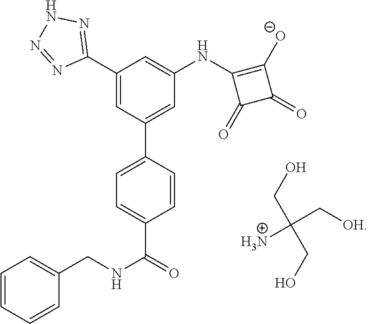 [SMILES: O=C(NCc1ccccc1)c1ccc(-c2cc(Nc3c([O-])c(=O)c3=O)cc(-c3nn[nH]n3)c2)cc1 ; [NH3+]C(CO)(CO)CO]

The present disclosure also provides crystalline forms of Compound A or a pharmaceutically acceptable salt thereof. In some embodiments, the crystalline form comprises Compound A free acid or Compound A tromethamine salt.

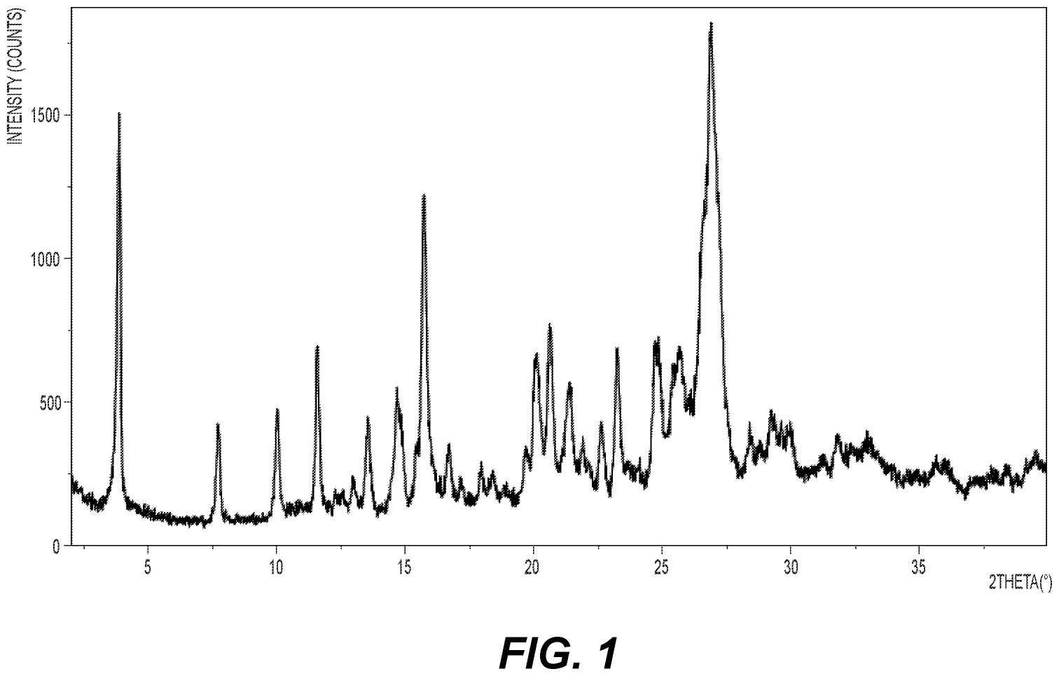

In some embodiments, the crystalline form comprises Compound A tromethamine salt. In some embodiments, the crystalline Compound A tromethamine salt is a hydrate. In one embodiment, the crystalline Compound A tromethamine salt is Hydrate I. In one embodiment, the crystalline Compound A tromethamine salt is characterized by an XRPD pattern substantially in accordance with FIG. 1. In one embodiment, the crystalline Compound A tromethamine salt is characterized by an XRPD pattern comprising diffraction angles at 3.9±0.2, 7.7±0.2, 10.0±0.2, and 15.8±0.2°2θ, when measured using Cu Kα radiation.

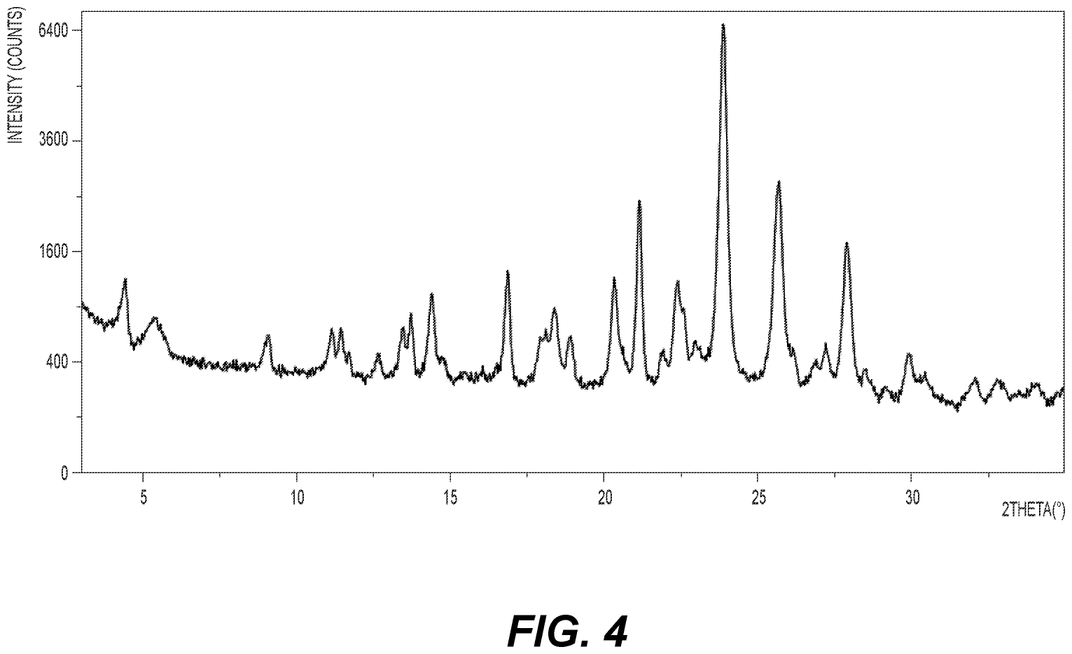

In one embodiment, the crystalline Compound A tromethamine salt is Hydrate II. In one embodiment, the crystalline Compound A tromethamine salt is characterized by an XRPD pattern substantially in accordance with FIG. 4. In one embodiment, the crystalline Compound A tromethamine salt is characterized by an XRPD pattern comprising diffraction angles at 4.4±0.2, 14.4±0.2, and 23.9±0.2°2θ, when measured using Cu Kα radiation.

In some embodiments, the crystalline form comprises Compound A (free acid). In some embodiments, the crystalline Compound A (free acid) is a hydrate. In some embodiments, the crystalline Compound A (free acid) is Pattern 1. In some embodiments, the crystalline Compound A (free acid) is Pattern 3.

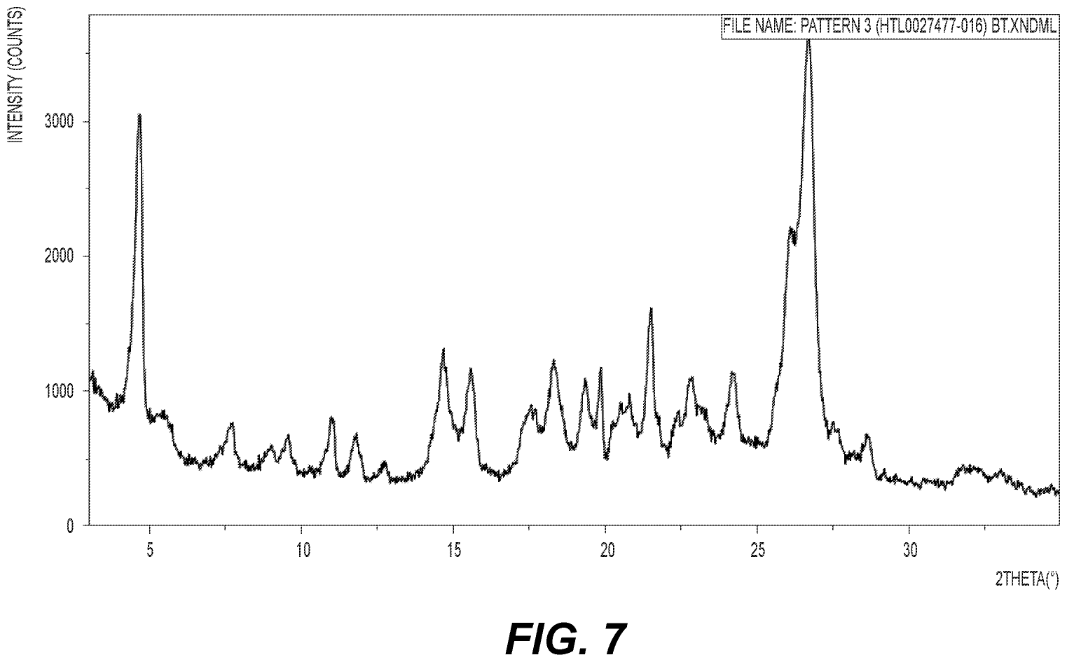

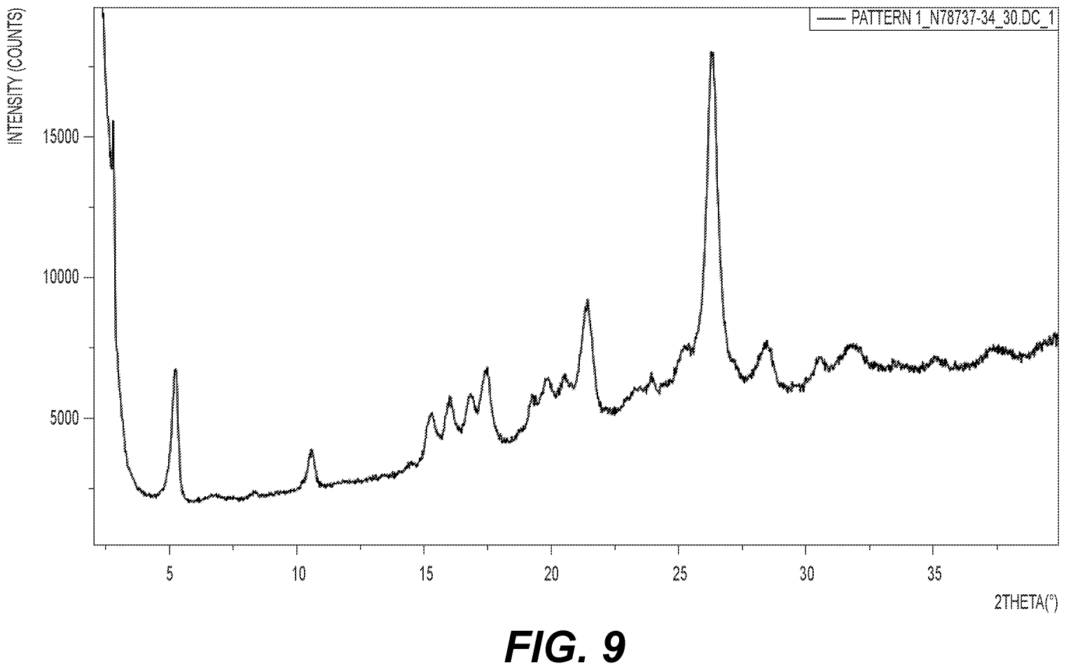

In one embodiment, the crystalline Compound A free acid is characterized by an XRPD pattern substantially in accordance with FIG. 7. In one embodiment, the crystalline Compound A free acid is characterized by an XRPD pattern substantially in accordance with FIG. 9.

The compounds disclosed herein can be used in therapy. The compounds disclosed herein can be used in medicine.

The compounds may be used as GPR35 receptor agonists. The compounds may be used in the manufacture of medicaments. The compounds may be for use in treating, preventing, ameliorating, controlling or reducing the risk of disorders associated with GPR35. The compounds may be used in the treatment or prevention of mast cell disorders, acute and chronic pain conditions and diseases associated with allergic or inflammatory diseases in both the gastrointestinal system and the lung.

The compounds may be used for treating gastrointestinal disorders and conditions, using agents that selectively act at GPR35 receptor. These include but are not limited to: food allergy, food intolerance and allergic disorders, celiac disease, gastrointestinal symptoms associated with systemic mastocytosis and other mast cell related disorders (mast cell activation syndrome, clonal mast cell disorder, monoclonal mast cell activation syndrome, idiopathic urticaria, idiopathic anaphylaxis), mastocytic colitis, irritable bowel syndrome (IBS), gastrointestinal motility disorders, functional gastrointestinal disorders, gastroesophageal reflux disease (GERD), duodenogastric reflux, diarrhoeal diseases, eosinophilic gastroenteritis, eosinophilic esophagitis, infectious diarrhea (such as Clostridium difficile, Salmonella, Shigella toxin), microscopic colitis, immune mediated gastrointestinal diseases, Crohn's disease, ulcerative colitis, inflammatory bowel disease, visceral abdominal pain. In some embodiments, the compounds may be used for treating irritable bowel syndrome (IBS), including IBS with constipation (IBS-C), IBS with diarrhea (IBS-D), and IBS with mixed bowel habits (IBS-M). In some embodiments, the compounds may be used for treating IBS-D.

In some embodiments, the compounds may be used for treating inflammatory bowel disease (IBD). In some embodiments, the compounds may be used for treating Crohn's disease. In some embodiments, the compounds may be used for treating ulcerative colitis.

The compounds may be used for treating the symptoms of pain associated with gastrointestinal disease and other visceral conditions including Crohn's disease, ulcerative colitis, inflammatory bowel disease, radiation colitis, radiation cystitis, celiac disease, gluten enteropathy, radiation cystitis, interstitial cystitis, painful bladder syndrome; cancer, gastroesophageal reflux disease, chemotherapy and radiotherapy mucositis, pancreatitis, prostatitis, pelvic pain, endometriosis, hepatitis; hepatic fibrosis and cirrhosis.

The compounds may be used for treating pulmonary diseases and conditions. These include but are not limited to chronic obstructive pulmonary diseases, asthma, chronic bronchitis, cystic fibrosis, emphysema, chronic idiopathic cough, hyperactive airway disorder and idiopathic pulmonary fibrosis.

The present application also provides a method of treatment according to any use of the compound of formula (1) described herein. In some embodiments, there is provided a method of treating disorders associated with GPR35 in a subject in need thereof, comprising administering to the subject a therapeutically effective amount of a compound of formula (1) (e.g., Compound A) or a pharmaceutically acceptable salt thereof. In some embodiments, the subject is a human.

In some embodiments, the disorder is inflammatory bowel disease (IBD). In some embodiments, the disorder is Crohn's disease. In some embodiments, the disorder is ulcerative colitis.

In some embodiments, the disorder is irritable bowel syndrome (IBS), including IBS with constipation (IBS-C), IBS with diarrhea (IBS-D), and IBS with mixed bowel habits (IBS-M). In some embodiments, the disorder is IBS-D.

### Definitions

In this application, the following definitions apply, unless indicated otherwise.

The term “treatment”, in relation to the uses of any of the compounds described herein, including those of the formula (1) is used to describe any form of intervention where a compound is administered to a subject suffering from, or at risk of suffering from, or potentially at risk of suffering from the disease or disorder in question. Thus, the term “treatment” covers both preventative (prophylactic) treatment and treatment where measurable or detectable symptoms of the disease or disorder are being displayed.

The term “effective therapeutic amount” (for example in relation to methods of treatment of a disease or condition) refers to an amount of the compound which is effective to produce a desired therapeutic effect. For example, if the condition is pain, then the effective therapeutic amount is an amount sufficient to provide a desired level of pain relief. The desired level of pain relief may be, for example, complete removal of the pain or a reduction in the severity of the pain.

The terms “alkyl” as in “C₁₋₆ alkyl”, “cycloalkyl” as in “C₃₋₆ cycloalkyl”, “alkoxy” as in “C₁₋₆ alkoxy”, “aryl”, “heteroaryl”, “monocyclic” and “bicyclic” are all used in their conventional sense (e.g. as defined in the IUPAC Gold Book), unless indicated otherwise.

To the extent that any of the compounds described have chiral centres, the present invention extends to all optical isomers of such compounds, whether in the form of racemates or resolved enantiomers. The invention described herein relates to all crystal forms, solvates and hydrates of any of the disclosed compounds however so prepared. To the extent that any of the compounds disclosed herein have acid or basic centres such as carboxylates or amino groups, then all salt forms of said compounds are included herein. In the case of pharmaceutical uses, the salt should be seen as being a pharmaceutically acceptable salt.

The compounds of the invention can exist in tautomeric forms. It is to be understood that any reference to a named compound or a structurally depicted compound is intended to encompass all tautomers of such compound. For example, the compound of formula (1) encompasses the tautomers shown below:

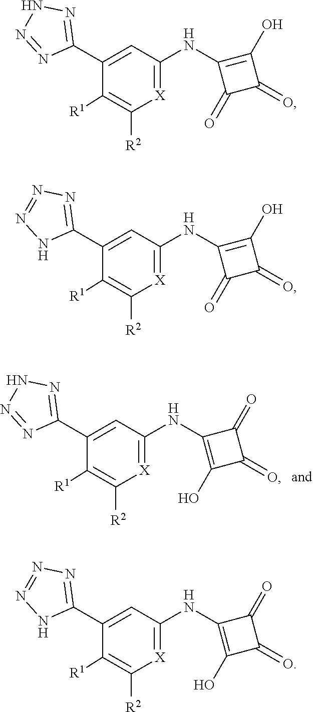

Salts or pharmaceutically acceptable salts that may be mentioned include acid addition salts and base addition salts. Such salts may be formed by conventional means, for example by reaction of a free acid or a free base form of a compound with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound in the form of a salt with another counter-ion, for example using a suitable ion exchange resin.

Examples of pharmaceutically acceptable salts include acid addition salts derived from mineral acids and organic acids, and salts derived from metals such as sodium, magnesium, potassium and calcium. Representative pharmaceutically acceptable base addition salts also include, but are not limited to, aluminium, ammonium, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc. In one embodiment, the salts of the compounds disclosed in the present disclosure are tromethamine (TRIS) salts.

Examples of acid addition salts include acid addition salts formed with acetic, 2,2-dichloroacetic, adipic, alginic, aryl sulfonic acids (e.g. benzenesulfonic, naphthalene-2-sulfonic, naphthalene-1,5-disulfonic and p-toluenesulfonic), ascorbic (e.g. L-ascorbic), L-aspartic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulfonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulfuric, tannic, tartaric (e.g. (+)-L-tartaric), thiocyanic, undecylenic and valeric acids.

Also encompassed are any solvates of the compounds and their salts. Preferred solvates are solvates formed by the incorporation into the solid-state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulfoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGA), differential scanning calorimetry (DSC) and X-ray crystallography.

The solvates can be stoichiometric or non-stoichiometric solvates. Particular solvates may be hydrates, and examples of hydrates include hemihydrates, monohydrates and dihydrates. For a more detailed discussion of solvates and the methods used to make and characterise them, see Bryn et al, Solid-State Chemistry of Drugs, Second Edition, published by SSCI, Inc of West Lafayette, IN, USA, 1999, ISBN 0-967-06710-3.

The term “pharmaceutical composition” in the context of this invention means a composition comprising an active agent and comprising additionally one or more pharmaceutically acceptable carriers. The composition may further contain ingredients selected from, for example, diluents, adjuvants, excipients, vehicles, preserving agents, fillers, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms. The compositions may take the form, for example, of tablets, dragees, powders, elixirs, syrups, liquid preparations including suspensions, sprays, inhalants, tablets, lozenges, emulsions, solutions, cachets, granules, capsules and suppositories, as well as liquid preparations for injections, including liposome preparations.

The compounds of the invention may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. In an analogous manner, a reference to a particular functional group also includes within its scope isotopic variations, unless the context indicates otherwise. For example, a reference to an alkyl group such as an ethyl group or an alkoxy group such as a methoxy group also covers variations in which one or more of the hydrogen atoms in the group is in the form of a deuterium or tritium isotope, e.g. as in an ethyl group in which all five hydrogen atoms are in the deuterium isotopic form (a perdeuteroethyl group) or a methoxy group in which all three hydrogen atoms are in the deuterium isotopic form (a trideuteromethoxy group). The isotopes may be radioactive or non-radioactive.

Therapeutic dosages may be varied depending upon the requirements of the patient, the severity of the condition being treated, and the compound being employed. Determination of the proper dosage for a particular situation is within the skill of the art. Generally, treatment is initiated with the smaller dosages which are less than the optimum dose of the compound. Thereafter the dosage is increased by small increments until the optimum effect under the circumstances is reached. For convenience, the total daily dosage may be divided and administered in portions during the day if desired.

The magnitude of an effective dose of a compound will, of course, vary with the nature of the severity of the condition to be treated and with the particular compound and its route of administration. The selection of appropriate dosages is within the ability of one of ordinary skill in this art, without undue burden. In general, the daily dose range may be from about 10 μg to about 30 mg per kg body weight of a human and non-human animal, preferably from about 50 μg to about 30 mg per kg of body weight of a human and non-human animal, for example from about 50 μg to about 10 mg per kg of body weight of a human and non-human animal, for example from about 100 μg to about 30 mg per kg of body weight of a human and non-human animal, for example from about 100 μg to about 10 mg per kg of body weight of a human and non-human animal and most preferably from about 100 μg to about 1 mg per kg of body weight of a human and non-human animal.

### Pharmaceutical Formulations

While it is possible for the active compound to be administered alone, it is preferable to present it as a pharmaceutical composition (e.g. formulation).

Accordingly, in some embodiments of the invention, there is provided a pharmaceutical composition comprising at least one compound of Formula (1) or a salt thereof as defined above together with at least one pharmaceutically acceptable excipient.

The pharmaceutically acceptable excipient(s) can be selected from, for example, carriers (e.g. a solid, liquid or semi-solid carrier), adjuvants, diluents (e.g solid diluents such as fillers or bulking agents; and liquid diluents such as solvents and co-solvents), granulating agents, binders, flow aids, coating agents, release-controlling agents (e.g. release retarding or delaying polymers or waxes), binding agents, disintegrants, buffering agents, lubricants, preservatives, anti-fungal and antibacterial agents, antioxidants, buffering agents, tonicity-adjusting agents, thickening agents, flavouring agents, sweeteners, pigments, plasticizers, taste masking agents, stabilisers or any other excipients conventionally used in pharmaceutical compositions.

The term “pharmaceutically acceptable” as used herein means compounds, materials, compositions, and/or dosage forms which are, within the scope of sound medical judgment, suitable for use in contact with the tissues of a subject (e.g. a human subject) without excessive toxicity, irritation, allergic response, or other problem or complication, commensurate with a reasonable benefit/risk ratio. Each excipient must also be “acceptable” in the sense of being compatible with the other ingredients of the formulation.

Pharmaceutical compositions containing compounds of the Formula (1) can be formulated in accordance with known techniques, see for example, Remington's Pharmaceutical Sciences, Mack Publishing Company, Easton, PA, USA. The pharmaceutical compositions can be in any form suitable for oral, parenteral, intravenous, intramuscular, intrathecal, subcutaneous, topical, intranasal, intrabronchial, sublingual, buccal, ophthalmic, otic, rectal, intra-vaginal, or transdermal administration.

Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

The composition may be a tablet composition or a capsule composition. Tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Tablets may be designed to release the drug either upon contact with stomach fluids (immediate release tablets) or to release in a controlled manner (controlled release tablets) over a prolonged period of time or with a specific region of the GI tract.

The pharmaceutical compositions typically comprise from approximately 1% (w/w) to approximately 95% (w/w) active ingredient and from 99% (w/w) to 5% (w/w) of a pharmaceutically acceptable excipient (for example as defined above) or combination of such excipients. Preferably, the compositions comprise from approximately 20% (w/w) to approximately 90% (w/w) active ingredient and from 80% (w/w) to 10% (w/w) of a pharmaceutically excipient or combination of excipients. The pharmaceutical compositions comprise from approximately 1% (w/w) to approximately 95% (w/w), preferably from approximately 20% (w/w) to approximately 90% (w/w), active ingredient. Pharmaceutical compositions according to the invention may be, for example, in unit dose form, such as in the form of ampoules, vials, suppositories, pre-filled syringes, dragees, powders, tablets or capsules.

Tablets and capsules may contain, for example, 0-20% (w/w) disintegrants, 0-5% (w/w) lubricants, 0-5% (w/w) flow aids and/or 0-99% (w/w) fillers/or bulking agents (depending on drug dose). They may also contain 0-10% (w/w) polymer binders, 0-5% (w/w) antioxidants, 0-5% (w/w) pigments. Slow release tablets would in addition typically contain 0-99% (w/w) release-controlling (e.g. delaying) polymers (depending on dose). The film coats of the tablet or capsule typically contain 0-10% (w/w) polymers, 0-3% (w/w) pigments, and/or 0-2% (w/w) plasticizers.

The composition may be a parenteral composition. Parenteral formulations may contain 0-20% (w/w) buffers, 0-50% (w/w) cosolvents, and/or 0-99% (w/w) Water for Injection (WFI) (depending on dose and if freeze dried). Formulations for intramuscular depots may also contain 0-99% (w/w) oils.

The composition may be in a form suitable for intranasal or intrabronchial administration. Such compositions should be suitable for atomisation, which allows inhalation through the mouth and facilitates absorption through the thin mucous membrane that lines the nasal passages.

Many drugs that are administered orally can also be administered rectally as a suppository. The composition may be in a form suitable for rectal administration. In this form, the composition may comprise a waxy substance that dissolves or liquefies after it is inserted into the rectum. Such compositions may be prescribed for people who cannot take a drug orally because they have nausea, cannot swallow, or have restrictions on eating, as is the case before and after many surgical operations.

The pharmaceutical formulations may be presented to a patient in “patient packs” containing an entire course of treatment in a single package, usually a blister pack.

The compounds of the Formula (1) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within these ranges, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 milligrams to 1 gram, of active compound.

In some embodiments, the pharmaceutical compositions comprise a crystalline form of Compound A free acid or Compound A tromethamine salt. In some embodiments, the pharmaceutical compositions comprise a crystalline form of Compound A free acid. In some embodiments, the pharmaceutical compositions comprise a crystalline form of Compound A tromethamine salt.

In some embodiments, the pharmaceutical compositions comprise Compound A tromethamine salt Hydrate I. In some embodiments, the pharmaceutical compositions comprise Compound A tromethamine salt Hydrate II. In some embodiments, the pharmaceutical compositions comprise Compound A tromethamine salt Hydrate I and Compound A tromethamine salt Hydrate I.

The active compound will be administered to a patient in need thereof (for example a human or animal patient) in an amount sufficient to achieve the desired therapeutic effect (effective amount). The precise amounts of compound administered may be determined by a supervising physician in accordance with standard procedures.

### Methods for the Preparation of Compounds of the Formula (1)

Compounds of the formula (1) can be prepared in accordance with synthetic methods as described herein, some of which will be known to the skilled person. The invention provides a process for the preparation of a compound as defined in formula (1) above.

Compounds of formula (1) may be prepared as described in Scheme 1 below.

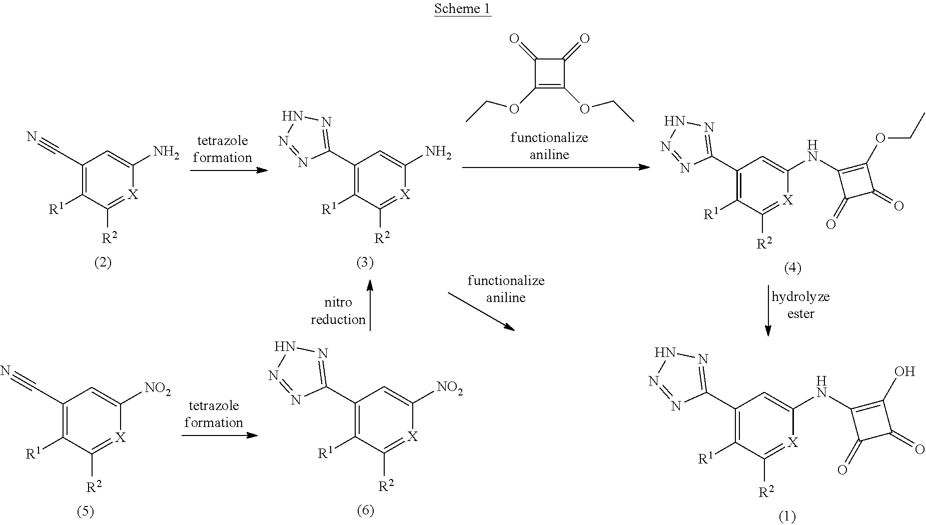

Thus, substituted amino aromatic nitriles of formula (2), which are either commercially available or readily accessible from commercial materials, are converted to tetrazole intermediates of formula (3), typically in the presence of sodium azide and ammonium chloride or zinc chloride, in solvents such as DMF, DMSO or 2-propanol, and heating to temperatures in the range 110-130° C. Alternatively, tetrazoles of formula (3) may be accessed from substituted 3-nitrobenzonitriles of formula (5), which are either commercially available or readily accessible from commercial materials. Conversion of the nitrile function to provide the corresponding tetrazoles of formula (6) is performed as above, and subsequent reduction of the nitro group is typically affected by zinc dust in the presence of ammonium chloride in 1,4-dioxane and water whilst heating to reflux. The aniline group of compounds of formula (3) is then functionalized by reaction with diethyl squarate in the presence of a base, typically triethylamine, in solvents such as EtOH or DCM. The ester function of squarates of formula (4) are then hydrolyzed, typically performed in a mixture of aqueous HCl and THF at mild temperatures, typically 60° C., to give Examples of the formula (1). Alternatively, substituted anilines of the formula (3) can be converted directly to Examples of the formula (1) by treatment with squaric acid, with conditions typically comprising water at reflux.

Alternatively, compounds of formula (1) may be prepared as described in Scheme 2 below. Aryl bromide intermediates of formula (8) can be prepared from amino anilines of the formula (7) as described in Scheme 1. Subsequent Suzuki reaction between aryl bromides of formula (8) and a suitable boronic acid or boronic ester coupling partner (R═H or alkyl) affords Examples of formula (1). The Suzuki reaction is typically carried out under microwave irradiation in the presence of a base, such as K₂CO₃, and a catalytic palladium source, such as [1,1′-Bis(di-tert-butylphosphino)ferrocene]dichloropalladium(II), in a suitable solvent system, typically a combination of MeCN and H₂O.

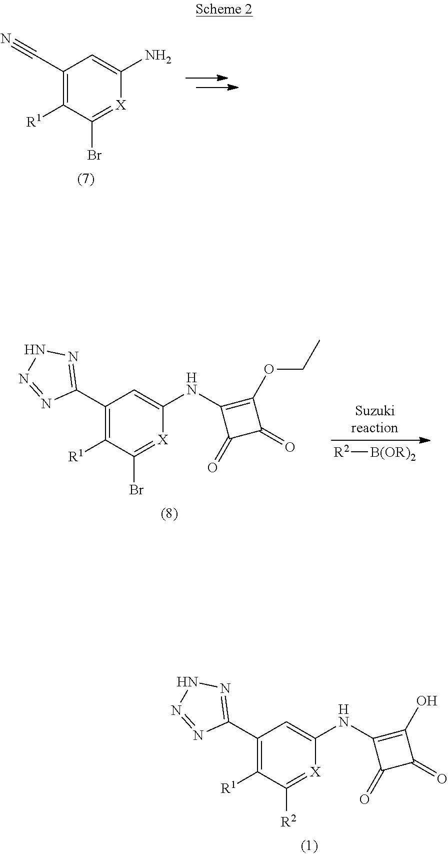

Scheme 3 shows a variation for preparation of compounds of formula (1), whereby R² is a phenyl optionally substituted with —CONHCH₂R⁴, —CONHCH₂CH₂OR⁴, —CONHR⁴ or —CON(CH₃)R⁴. Biaryl carboxylic acid compounds of formula (9) are readily accessible from commercial 3-amino-5-bromobenzonitrile (7) and are converted to the corresponding tetrazole compounds of formula (10) as described in Scheme 1. The aniline group of intermediate compounds of formula (10) are then functionalized as described in Scheme 1. The carboxylic acid group of compounds of formula (11) are then converted to amides in the presence of a suitable amine, coupling agent such as 1-[bis(dimethylamino)methylene]-1H-1,2,3-triazolo[4,5-b]pyridinium 3-oxid hexafluorophosphate, a base such as N,N-diisopropylethylamine and a solvent such as DMF. The ester function of cyclobut-3-ene-1,2-diones of formula (12) is then hydrolyzed to afford Examples of formula (13) (corresponding to formula (1) whereby R² is a phenyl optionally substituted with —CONHCH₂R⁴, —CONHCH₂CH₂OR⁴, —CONHR⁴ or —CON(CH₃)R⁴).

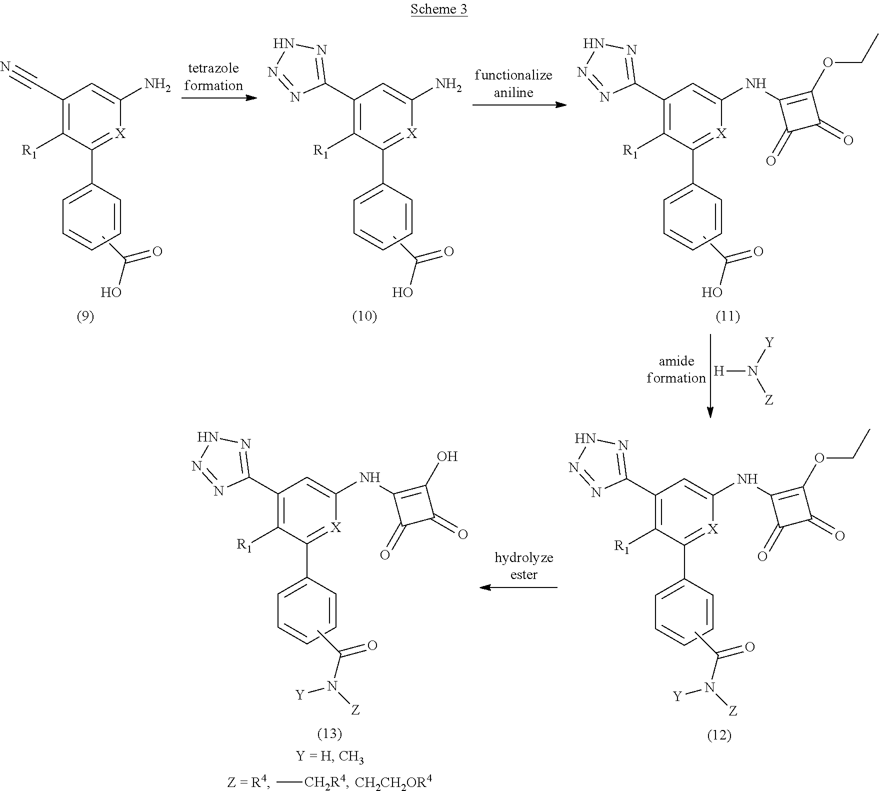

In circumstances whereby X is N, and R¹ is H, compounds of formula (2b) may be prepared as described in Scheme 4. Accordingly, commercial 2,6-dichloroisonicotinonitrile (14) is subject to an S_{N}Ar reaction with (4-methoxyphenyl)methanamine in a solvent such as NMP with heating to temperatures in the range 110-120° C. The nitrile function of intermediates of formula (15) are then converted to tetrazole groups as described in Scheme 1. Where R² is an alkoxy or phenoxy group, the chlorine group of pyridine (16) is displaced by a suitable alcohol or metal alkoxide (e.g. NaOMe) in the presence or absence of a base such as K₂CO₃, in a solvent such as DMSO and at temperatures varying from room temperature to 120° C. to give alkoxy or phenoxy substituted compounds (17). Alternatively, when R² is phenyl, a Suzuki reaction between pyridyl chloride of formula (16) and the appropriate phenyl boronic acid or ester affords intermediate (17). The Suzuki reaction is typically carried out in the presence of a base such as K₂CO₃, and a catalytic source of palladium, typically [1,1′-Bis(diphenylphosphino)ferrocene]dichloropalladium(II), in a solvent such as 1,4-dioxane, at temperatures of 120° C. It will be understood by the skilled person that the order of steps as laid out in Scheme 4 may be completed in a different sequence to that shown, without affecting the overall success of the synthesis of the desired compound of formula (2b). Deprotection of the amino function of compounds of formula (17) is then achieved by treatment with acid, typically TFA at 70° C. Conversion of Intermediates of formula (18) to give Examples of formula (2b) is then achieved as described in Scheme 1.

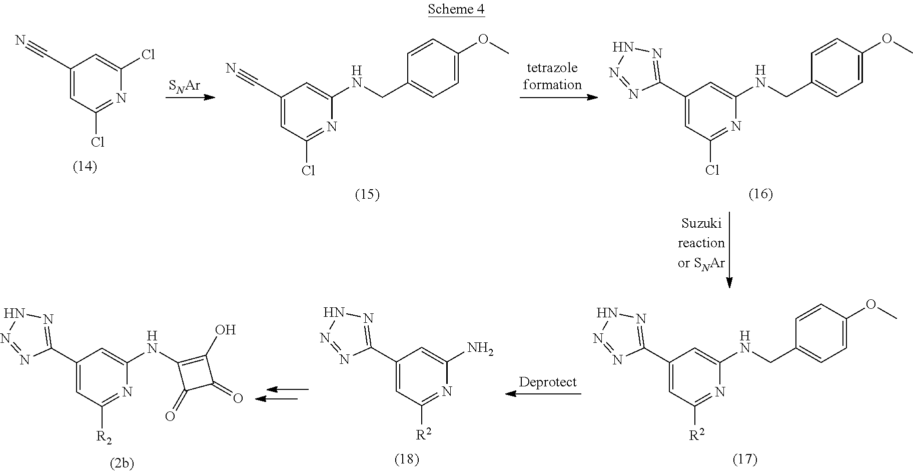

In certain reactions described above, it may be necessary to protect one or more groups to prevent reaction from taking place at an undesirable location on the molecule. Examples of protecting groups, and methods of protecting and deprotecting functional groups, can be found in Greene's Protective Groups in Organic Synthesis, Fifth Edition, Editor: Peter G. M. Wuts, John Wiley, 2014, (ISBN:9781118057483).

Compounds made by the foregoing methods may be isolated and purified by any of a variety of methods well known to those skilled in the art and examples of such methods include recrystallisation and chromatographic techniques such as column chromatography (e.g. flash chromatography), HPLC and SFC.

### General Procedures

Where no preparative routes are included, the relevant intermediate is commercially available. Commercial reagents were utilized without further purification. Final compounds and intermediates are named using ChemDraw Professional, Version 17.0.0.206 (121). Room temperature (RT) refers to approximately 20-27° C. 1H NMR spectra were recorded at 400 or 500 MHz on either a Bruker, Varian or Jeol instrument. Chemical shift values are expressed in parts per million (ppm), i.e. (δ)—values relative to the following solvents: chloroform-d=7.26 ppm, DMSO-d6=2.50 ppm, methanol-d4=3.31 ppm. The following abbreviations are used for the multiplicity of the NMR signals: s=singlet, br=broad, d=doublet, t=triplet, q=quartet, m=multiplet. Coupling constants are listed as J values, measured in Hz. NMR and mass spectroscopy results were corrected to account for background peaks. Chromatography refers to column chromatography performed using 60-120 mesh or 40-633 μm, 60 Å silica gel and executed under nitrogen pressure (flash chromatography) conditions. Microwave-mediated reactions were performed in Biotage Initiator or CEM Discover microwave reactors.

### LCMS Analysis

LCMS analysis of compounds was performed under electrospray conditions using the instruments and methods given below:

### LCMS Method A

Instruments: HP 1100 with G1315A DAD, Micromass ZQ; Column: Phenomenex Gemini-NX C-18, 3 micron, 2.0×30 mm; Gradient [time (min)/solvent B in A (%)]: 0.00/2, 0.10/2, 2.50/95, 3.50/95; Solvents: solvent A=2.5 L H₂O+2.5 mL 28% ammonia in H₂O solution; solvent B=2.5 L MeCN+135 mL H₂O+2.5 mL 28% ammonia in H₂O solution. Injection volume 1 μL; UV detection 230 to 400 nm; Mass detection 130 to 800 AMU; column temperature 45° C.; Flow rate 1.5 mL/min.

### LCMS Method B

Instruments: Agilent 1260 Infinity LC with Diode Array Detector, Agilent 6120B Single Quadrupole MS with API-ES Source; Column: Phenomenex Gemini-NX C-18, 3 micron, 2.0×30 mm; Gradient [time (min)/solvent B in A (%)]: 0.00/5, 2.00/95, 2.50/95, 2.60/5, 3.00/5; Solvents: solvent A=2.5 L H₂O+2.5 mL 28% NH₃ in H₂O; solvent B=2.5 L MeCN+129 mL H₂O+2.7 mL of (28% NH₃ in H₂O); Injection volume 0.5 μL; UV detection 190 to 400 nm; Mass detection 130 to 800 AMU; column temperature 40° C.; Flow rate 1.5 mL/min.

### LCMS Methods C and D

Instruments: Agilent 1260 Infinity LC with Diode Array Detector, Agilent 6120B Single Quadrupole MS with API-ES Source; Column: Restek, Penta Fluoro Phenyl Propyl, 3 micron, 2.1×30 mm. Gradient [time (min)/solvent B in A (%)]: Method C: 0.00/5, 2.00/95, 2.50/95, 2.60/5, 3.00/5 or Method D: 0.00/2, 0.1/2, 8.4/95, 10/95, 10.1/2, 12/2; Solvents: solvent A=water (2.5 L) with 2.5 mL Formic acid; Solvent B=MeCN (2.5 L) with 125 mL water and 2.5 mL Formic acid. Injection volume 0.5 μL; UV detection 190 to 400 nm; Mass detection 130 to 800 AMU; column temperature 40° C.; Flow rate 1.5 mL/min.

### LCMS Method E

Instruments: Waters Acquity H Class, Photo Diode Array, SQ Detector; Column: BEH C18, 1.7 micron, 2.1×50 mm; Gradient [time (min)/solvent B in A (%)]: 0.00/5, 0.40/5, 0.8/35, 1.20/55, 2.50/100, 3.30/100 4.00/5; Solvents: solvent A=5 mM mmmonium acetate and 0.1% formic acid in H₂O; solvent B=0.1% formic acid in MeCN; Injection volume 2 μL; UV detection 200 to 400 nm; Mass detection 100 to 1200 AMU; column at ambient temperature; Flow rate 0.55 mL/min.

### LCMS Method F

Instruments: Agilent 1100 Series with DAD/ELSD, Agilent LC\MSD VL (G1956A), SL (G1956B) mass-spectrometer; Column: Zorbax SB-C18, 1.8 micron, 4.6×15 mm; Gradient [time (min)/solvent B in A (%)]: 0.0/0, 1.5/100, 1.8/100, 1.81/0; Solvents: solvent A=water and 0.1% formic acid; solvent B=MeCN and 0.1% formic acid; Injection volume 1 μL; UV detection 200 to 400 nm; Mass detection 80-1000 AMU; column at ambient temperature; Flow rate 3.0 mL/min.

### LCMS Method G

Instruments: Waters 2690 with PDA Detector 996, Acquity QDA mass-spectrometer; Column: X-BRIDGE C18, 5.0 micron, 4.6×100 mm; Gradient [time (min)/solvent B in A (%)]: 0.0/10, 1.0/10, 5.0/100, 7.0/100, 7.50/10, 8.0/10; Solvents: solvent A=0.1% formic acid and 10 mM ammonium bicarbonate in water; solvent B=MeCN; Injection volume 1 μL; UV detection 190 to 800 nm; Mass detection 30-1250 AMU; column temperature 35° C.; Flow rate 1.2 mL/min.

### LCMS Method H

Instruments: Waters Acquity H Class with photo diode array, SQ detector mass-spectrometer; Column: BEH C18, 1.7 micron, 2.1×50 mm; Gradient [time (min)/solvent B in A (%)]: 0.0/5, 0.4/5, 0.8/35, 1.2/55, 2.5/100, 3.3/100, 3.31/5, 4.0/5; Solvents: solvent A=0.1% formic acid and 5 mM ammonium acetate in water; solvent B=0.1% formic acid in MeCN; Injection volume 1 μL; UV detection 200 to 400 nm; Mass detection 100-1200 AMU; column at ambient temperature; Flow rate 0.55 mL/min.

### LCMS Methods I AND J

Instruments for Method I: Shimadzu Nexera with photo diode array, LCMS-2020 mass-spectrometer or Instruments for Method J: Agilent 1290 RRLC with photo diode array, Agilent 6120 mass-spectrometer; Column: X-BRIDGE C18, 3.5 micron, 4.6×50 mm; Gradient [time (min)/solvent B in A (%)]: 0.0/5, 5.0/90, 5.8/95, 7.20/95, 7.21/5, 10.0/5; Solvents: solvent A=0.1% ammonia in water; solvent B=0.1% ammonia in MeCN; Injection volume 1 μL; UV detection 200 to 400 nm; Mass detection 60-1000 AMU; column at ambient temperature; Flow rate 1.0 mL/min.

### LCMS Method K

Instruments: Waters 2690 with PDA Detector 996, Acquity QDA mass-spectrometer; Column: X-BRIDGE C18, 5.0 micron, 4.6×100 mm; Gradient [time (min)/solvent B in A (%)]: 0.0/10, 3.0/10, 6.0/100, 7.0/100, 7.01/10, 10.0/10; Solvents: solvent A=0.1% formic acid in water; solvent B=MeOH; Injection volume 1 μL; UV detection 190 to 800 nm; Mass detection 30-1250 AMU; column temperature 35° C.; Flow rate 1.0 mL/min.

### LCMS Method L

Instruments: Shimadzu LCMS-2010 EV with Shimadzu SPD-M20A PDA, single quadrupole mass-spectrometer; Column: Atlantis C18, 3.0 micron, 4.6×50 mm; Gradient [time (min)/solvent B in A (%)]: 0.0/30, 3.0/90, 6.0/90, 6.1/30; Solvents: solvent A=0.1% formic acid in water; solvent B=MeCN; Injection volume 1 μL; UV detection 254 nm; Mass detection 80-800 AMU; column temperature 40° C.; Flow rate 0.8 mL/min.

### LCMS Method M

Instruments: Shimadzu LCMS-2010 EV with Shimadzu SPD-M20A PDA, single quadrupole mass-spectrometer; Column: Capcell pack C18, 3.0 micron, 4.6×150 mm; Gradient [time (min)/solvent B in A (%)]: 0.0/30, 5.0/98, 9.5/98, 11.5/3, 12/3; Solvents: solvent A=0.1% formic acid in water; solvent B=MeCN; Injection volume 1 μL; UV detection 254 nm; Mass detection 80-800 AMU; column temperature 40° C.; Flow rate 0.8 mL/min.

### Abbreviations

Bn=benzyl
CDI=carbonyldiimidazole
DCM=dichloromethane
DIPEA=N,N-diisopropylethylamine
DMF=dimethylformamide
DMSO=dimethyl sulfoxide
ESI=electro spray ionisation
EtOAc=ethyl acetate
EtOH=ethanol
h=hour(s)
HATU=(1-[Bis(dimethylamino)methylene]-1H-1,2,3-triazolo[4,5-b]pyridinium 3-oxide
hexafluorophosphate
H₂O=water
HCl=hydrogen chloride, hydrochloric acid
(prep) HPLC=(preparative) high performance liquid chromatography
IPA=propan-2-ol
K₂CO₃=potassium carbonate
LC=liquid chromatography
MeCN=acetonitrile
MeOH=methanol
min(s)=minute(s)
MS=mass spectrometry
nm=nanometre(s)
NMP=N-methyl-2-pyrrolidone
NMR=nuclear magnetic resonance
POCl₃=phosphorus oxychloride
RT=room temperature
sat.=saturated
SFC=supercritical fluid chromatography
TEA=triethylamine
TFA=trifluoroacetic acid
THF=tetrahydrofuran
TLC=thin layer chromatography

### General Synthetic Procedures for the Intermediates

### Route 1

### Procedure for the Preparation of Intermediate 3, 5-amino-[1,1′-biphenyl]-3-carbonitrile

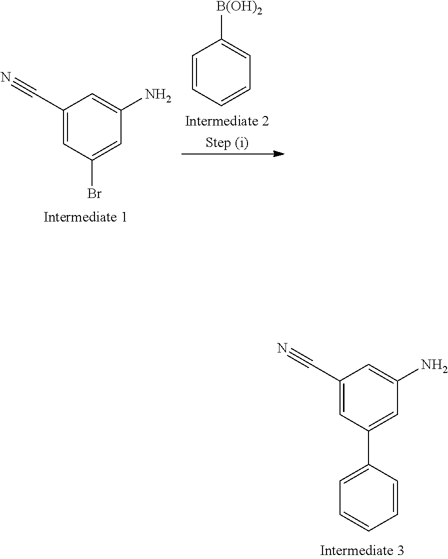 [0203] Step (i): A solution of Intermediate 1, 3-amino-5-bromobenzonitrile (200 mg, 1.02 mmol), Intermediate 2, phenylboronic acid (161 mg, 1.32 mmol), K₂CO₃ (281 mg, 2.03 mmol) and Pd(Ph₃P)₄ (60 mg, 0.05 mmol) in 1,4-dioxane (4 mL) and water (1 mL) was heated to 100° C. under microwave irradiation for 3 hours, after which it was diluted with EtOAc and water. The organics were separated, washed with brine, dried via passage through a hydrophobic frit and concentrated. The crude material was purified by flash column chromatography (normal phase, silica) under a gradient of EtOAc (0% to 45%) in PE to afford Intermediate 3, 5-amino-[1,1′-biphenyl]-3-carbonitrile (165 mg, 0.85 mmol, 85% yield) as an orange solid. Data available in Table 2.

### Route 2

### Procedure for the Preparation of Intermediate 5, ethyl 3′-amino-5′-(1H-tetrazol-5-yl)-[1,1′-biphenyl]-3-carboxylate

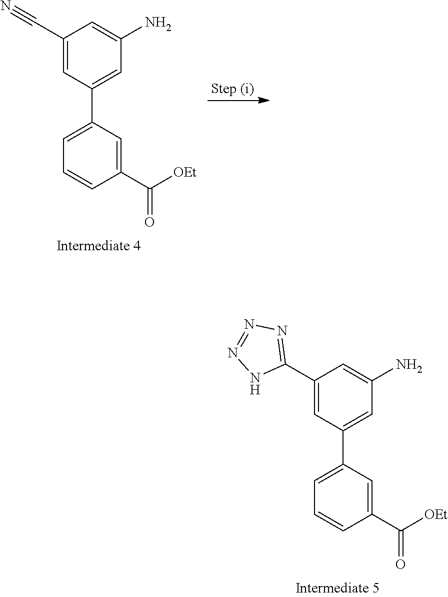 [SMILES: CCOC(=O)c1cccc(-c2cc(N)cc(C#N)c2)c1 ; "Step (i)" ; CCOC(=O)c1cccc(-c2cc(N)cc(-c3nnn[nH]3)c2)c1] [0205] Step (i): To a solution of Intermediate 4, ethyl 3′-amino-5′-cyano-[1,1′-biphenyl]-3-carboxylate (73 mg, 0.27 mmol) in DMF (0.5 mL) were added NaN₃ (36 mg, 0.55 mmol) and NH₄Cl (30 mg, 0.55 mmol). The mixture was heated to 130° C. for 3 hours, after which it was cooled to RT and acidified to pH 3-4 with 1 M HCl (aq). The precipitate was collected by filtration and dried under vacuum to afford Intermediate 5, ethyl 3′-amino-5′-(1H-tetrazol-5-yl)-[1,1′-biphenyl]-3-carboxylate (76 mg, 0.25 mmol, 91% yield) as a peach solid. Data available in Table 2.

### Route 3

### Procedure for the Preparation of Intermediate 12, N-benzyl-3′-((2-ethoxy-3,4-dioxocyclobut-1-en-1-yl)amino)-5′-(1H-tetrazol-5-yl)-[1,1′-biphenyl]-4-carboxamide

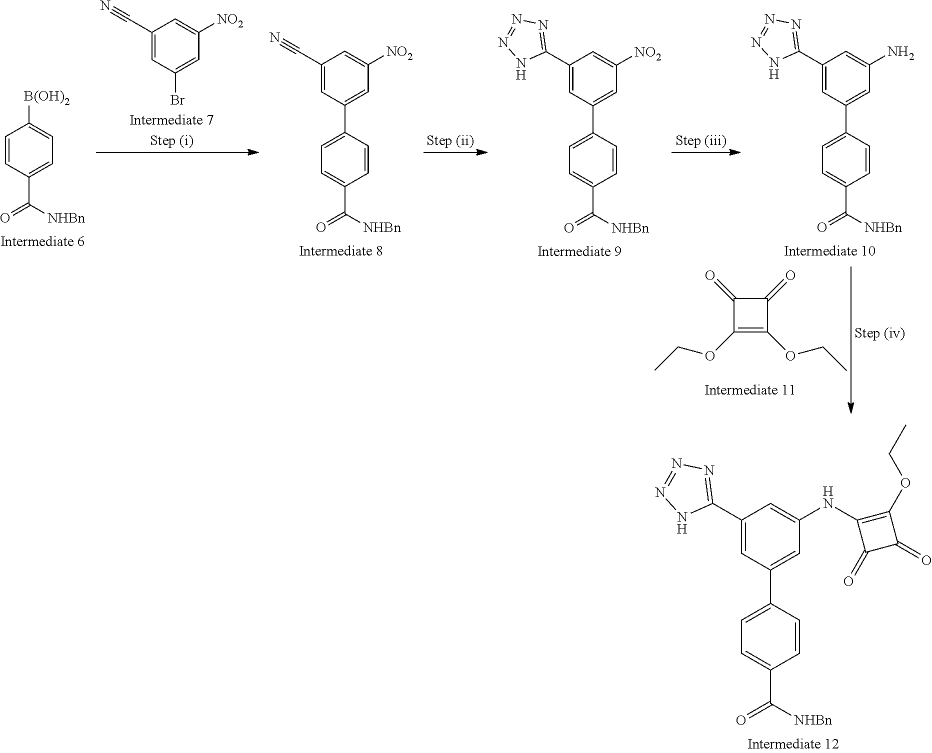 [0207] Step (i): A mixture of Intermediate 7, 3-Bromo-5-nitrobenzonitrile (1.0 g, 4.41 mmol), Intermediate 6, 4-(N-Benzylaminocarbonyl)phenylboronic acid (1.24 g, 4.85 mmol), K₂CO₃ (1.22 g, 8.81 mmol) and Pd(Ph₃P)₄ (0.15 g, 0.130 mmol) in 1,4-Dioxane (17 mL) and Water (2 mL) was heated to 100° C. under microwave irradiation for 1 hour. After this time the mixture was diluted with EtOAc, washed with water then brine, dried via passage through a hydrophobic frit and concentrated. The crude material was triturated with Et₂O and the resulting solid was collected by filtration to afford Intermediate 8, N-benzyl-3′-cyano-5′-nitro-[1,1′-biphenyl]-4-carboxamide (1.12 g, 3.13 mmol, 71% yield) as a tan solid. Data available in Table 2.
Step (ii): A mixture of Intermediate 8, N-benzyl-4-(3-cyano-5-nitro-phenyl)benzamide (1.03 g, 2.87 mmol), NaN₃ (373 mg, 5.74 mmol) and NH₄Cl (307 mg, 5.74 mmol) in DMF (22 mL) was heated to 130° C. for 2 hours. The mixture was cooled to RT and acidified to pH 2 with 1 M HCl (aq). The solid was collected by filtration, suspended in toluene and concentrated (×2) to afford Intermediate 9, N-benzyl-4-[3-nitro-5-(1H-tetrazol-5-yl)phenyl]benzamide (950 mg, 2.37 mmol, 83% yield) as a tan solid. Data available in Table 2.
Step (iii): A mixture of Intermediate 9, N-benzyl-4-[3-nitro-5-(1H-tetrazol-5-yl)phenyl]benzamide (630 mg, 1.57 mmol), zinc dust (412 mg, 6.29 mmol) and NH₄Cl (842 mg, 15.7 mmol) in 1,4-Dioxane (44 mL) and Water (6.3 mL) was heated to reflux for 4 hours. The mixture was cooled to RT and filtered through a bed of Celite (washing through with MeOH). The filtrate was concentrated and the residue was dissolved in 1 M HCl (aq) (20 mL). The pH was adjusted to ˜6 by the addition of 1 M NaOH (aq). The precipitate was collected by filtration, washed with water then suspended in toluene and concentrated to afford Intermediate 10, 4-[3-amino-5-(1H-tetrazol-5-yl)phenyl]-N-benzyl-benzamide (575 mg, 1.55 mmol, 99% yield) as a tan solid. Data available in Table 2.
Step (iv): A suspension of Intermediate 10, 4-[3-amino-5-(1H-tetrazol-5-yl)phenyl]-N-benzyl-benzamide (575 mg, 1.55 mmol), Intermediate 11, diethyl squarate (423 mg, 2.48 mmol) and TEA (0.44 mL, 3.1 mmol) in EtOH was stirred at RT for 24 hours, after which 1 M HCl (aq) was added. The precipitate was collected by filtration, suspended in toluene and concentrated to give Intermediate 12, N-benzyl-3′-((2-ethoxy-3,4-dioxocyclobut-1-en-1-yl)amino)-5′-(1H-tetrazol-5-yl)-[1,1′-biphenyl]-4-carboxamide (635 mg, 1.28 mmol 83% yield) as a tan solid. Data available in Table 2.

### Route 4

### Procedure for the Preparation of Intermediate 15, 3′-amino-5′-cyano-N-(2-methoxyethyl)-[1,1′-biphenyl]-4-carboxamide

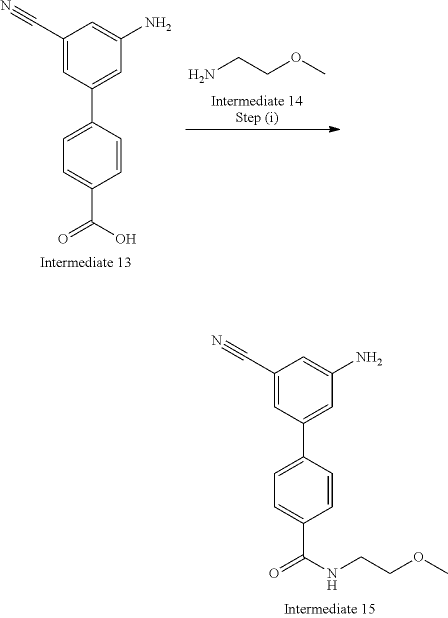 [0212] Step (i): To a solution of Intermediate 13, 3′-amino-5′-cyano-[1,1′-biphenyl]-4-carboxylic acid (400 mg, 1.68 mmol), Intermediate 14, 2-methoxyethan-1-amine (0.3 mL, 3.36 mmol), and DIPEA (0.88 mL, 5.04 mmol) in DMF (4.8 mL) was added HATU (768 mg, 2.02 mmol). The mixture was stirred at RT for 1 hour after which it was diluted with EtOAc, washed with water and brine, dried via passage through a hydrophobic frit and concentrated. The crude material was purified by flash column chromatography (reversed phase, C18) under a gradient of MeCN (10% to 50%) in water (containing 0.1% v/v HCOOH) to afford Intermediate 15, 3′-amino-5′-cyano-N-(2-methoxyethyl)-[1,1′-biphenyl]-4-carboxamide (317 mg, 1.07 mmol, 64% yield) as a tan solid. Data available in Table 2.

### Route 5

### Procedure for the Preparation of Intermediate 17, 4-chloro-3-(1H-tetrazol-5-yl)aniline

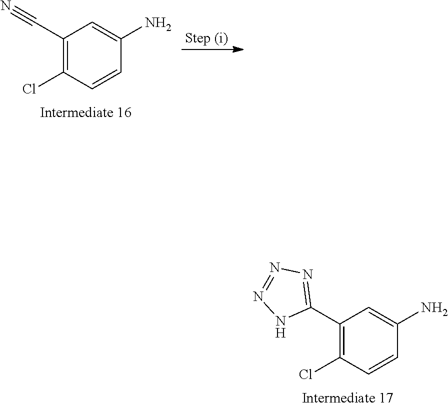 [0214] Step (i): A solution of Intermediate 16, 5-amino-2-chlorobenzonitrile (400 mg, 2.63 mmol), NaN₃ (342 mg, 5.26 mmol), NH₄Cl (281 mg, 5.88 mmol) in DMF (3 mL) was heated to 130° C. for 3 days. The mixture was cooled, and the insoluble material was removed via filtration, washing with MeCN. The solution was concentrated under reduced pressure, the residue was dissolved in EtOAc and washed with water. The organic layer was dried and the solvent was evaporated. The residue was crystallized from DCM to afford Intermediate 17, 4-chloro-3-(1H-tetrazol-5-yl)aniline. Data available in Table 2.

### Route 6

### Procedure for the Preparation of Intermediate 21, 3-cyclopropyl-5-(1H-tetrazol-5-yl)aniline

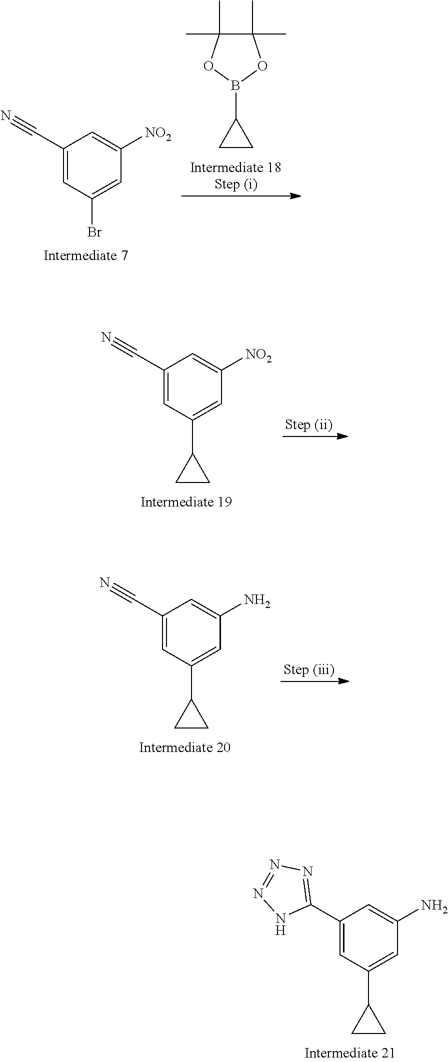 [0216] Step (i): Intermediate 7, 3-bromo-5-nitrobenzonitrile (3.00 g, 13.3 mmol), Intermediate 18, (2-cyclopropyl-4,4,5,5-tetramethyl-1,3,2-dioxaborolane) (3.34 g, 19.9 mmol) and K₂CO₃ (3.68 g, 26.62 mmole) were suspended in 1,4-dioxane (22 mL) and water (8 mL) at room temperature and degassed with N₂ for 15 min. After this, PdCl₂(dppf) (0.97 g, 1.32 mmol) was added and the mixture was heated to 100° C. for 4 hours. The mixture was cooled and partitioned between water (300 mL) and EtOAc (200 mL). The organic layer was separated, and the aqueous layer was further extracted with EtOAc (2×100 mL). The combined organic layers were dried over Na₂SO₄ and concentrated. The crude material was purified by flash column chromatography (normal phase, silica) under a gradient of EtOAc (0% to 5%) in PE to afford Intermediate 19, 3-cyclopropyl-5-nitrobenzonitrile (1.30 g, 6.91 mmol, 52% yield) as a yellow solid. Data available in Table 2.
Step (ii): Intermediate 19, 3-cyclopropyl-5-nitrobenzonitrile (1.30 g, 6.91 mmol) was dissolved MeOH (7 mL) and a suspension of NH₄Cl (2.01 g, 37.2 mmol) and zinc powder (2.43 g, 37.22 mmole) in water (7 mL) was added. The reaction was stirred at RT for 30 min, after which it was filtered through Celite. The filtrate was concentrated, and the residue was partitioned between sat. aq. NaHCO₃ and EtOAc. The organic layer was separated, and the aqueous layer was further extracted with EtOAc (×2). The combined organic layers were dried over Na₂SO₄ and concentrated. The crude material was purified by flash column chromatography (normal phase, silica) under a gradient of EtOAc (0% to 30%) in PE to afford Intermediate 20, 3-amino-5-cyclopropylbenzonitrile (1.01 g, 6.39 mmol, 93% yield) as a yellow liquid. Data available in Table 2.
Step (iii): A suspension of Intermediate 20, 3-amino-5-cyclopropylbenzonitrile (1.01 g, 6.39 mmol), NaN₃ (2.07 g, 31.9 mmol) and ZnCl₂ (4.35 g, 31.9 mmol) in 2-propanol was heated to 130° C. for 16 hours. The mixture was cooled, filtered through Celite and the filtrate was concentrated. The residue was partitioned between water and EtOAc. The organic layer was separated, and the aqueous layer was further extracted with EtOAc (×2). The combined organic layers were dried over Na₂SO₄ and concentrated. The crude material was purified by flash column chromatography (reversed phase, C18) under a gradient of 0.1 N HCOOH aq. (0% to 30%) in MeCN to afford Intermediate 21, 3-cyclopropyl-5-(1H-tetrazol-5-yl)aniline (800 mg, 3.98 mmol, 62% yield). Data available in Table 2.

### Route 7

### Procedure for the Preparation of Intermediate 23, 3-amino-5-cyclohexylbenzonitrile

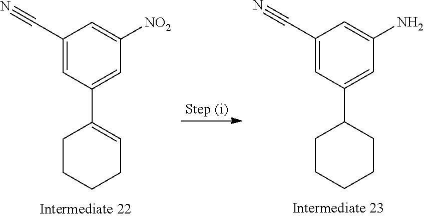 [SMILES: N#Cc1cc(C2=CCCCC2)cc([N+](=O)[O-])c1 ; "Step (i)" ; N#Cc1cc(N)cc(C2CCCCC2)c1] [0220] Step (i): A solution of Intermediate 22, 5-nitro-2′, 3′, 4′, 5′-tetrahydro-[1, 1′-biphenyl]-3-carbonitrile (3.00 g, 13.2 mmol) and 10% Pd/C, 50% moisture (3.00 g) in MeOH (30 mL) was stirred under an atmosphere of H₂ gas at RT for 5 hours. After this time, the mixture was filtered through Celite and concentrated. The crude material was purified by flash column chromatography (normal phase, silica) under a gradient of EtOAc (0% to 25%) in DCM to afford Intermediate 23, 3-amino-5-cyclohexylbenzonitrile (1.20 g, 6.0 mmol, 45% yield) as yellow liquid. Data available in Table 2.

### Route 8

### Procedure for the Preparation of Intermediate 25, 3-nitro-5-(pyridazin-4-yl)benzonitrile

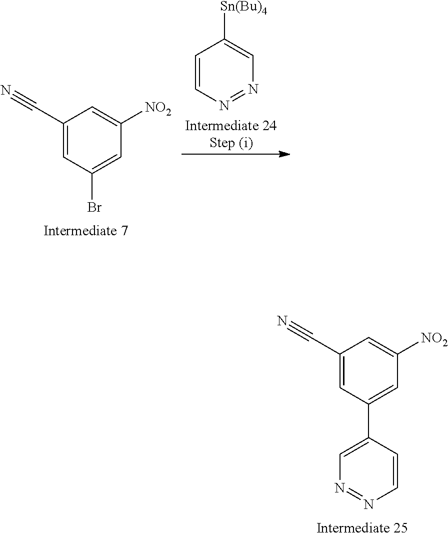 [0222] Step (i): A suspension of Intermediate 7, 3-bromo-5-nitrobenzonitrile (0.35 g, 1.55 mmol), Intermediate 24, 4-(tributylstannyl)pyridazine (0.57 g, 1.55 mmol) and CsF (0.47 g, 3.10 mmol) in MeCN was degassed with N₂ for 15 minutes, after which Pd(Ph₃P₄) (0.09 g, 0.80 mmol) and copper (1) iodide (0.06 g, 0.31 mmol) were added. The mixture was heated to 40° C. for 4 hours, after which it was cooled and partitioned between EtOAc and water. The organics were separated, and the aqueous layer was extracted with EtOAc. The combined organics were dried over Na₂SO₄ and concentrated. The crude material was purified by flash column chromatography (normal phase, silica) under a gradient of EtOAc (0% to 40%) in hexane to afford Intermediate 25, 3-nitro-5-(pyridazin-4-yl)benzonitrile (0.25 g, 1.10 mmol, 71%) as a yellow solid. Data available in Table 2.

### Route 9

### Procedure for the Preparation of Intermediate 29, 3′-((2-ethoxy-3,4-dioxocyclobut-1-en-1-yl)amino)-5′-(1H-tetrazol-5-yl)-[1,1′-biphenyl]-4-carboxylic acid

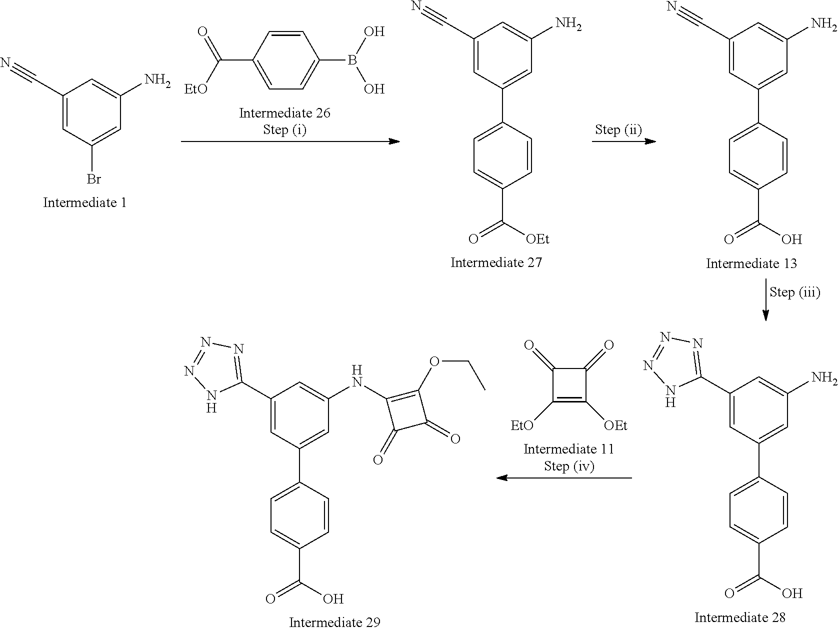 [0224] Step (i): A solution of Intermediate 1, 3-amino-5-bromobenzonitrile (20.0 g, 101.5 mmol), Intermediate 26, (4-(ethoxycarbonyl) phenyl) boronic acid (23.6 g, 121.8 mmol), K₂CO₃ (28.0 g, 203 mmol) and Pd(Ph₃P)₄ (2.34 g, 2.03 mmol) in in 1,4-Dioxane (200 mL) and water (30 mL) was heated to 90° C. for 16 hours, after which it was cooled and partitioned between EtOAc and water. The organics were separated, washed with brine, dried over Na₂SO₄ and concentrated. The crude product was purified by flash column chromatography (normal phase, silica) under a gradient of EtOAc (0% to 16%) in hexane to afford Intermediate 27, ethyl 3′-amino-5′-cyano-[1,1′-biphenyl]-4-carboxylate (23.0 g, 86.5 mmol, 85% yield) as an off-white solid. Data available in Table 2.
Step (ii): To a solution of Intermediate 27, ethyl 3′-amino-5′-cyano-[1,1′-biphenyl]-4-carboxylate (23.0 g, 86.5 mmol) in THF (130 mL) and water (130 mL) was added lithium hydroxide monohydrate (6.22 g, 259.3 mmol). The mixture was stirred at RT for 6 hours, after which it was concentrated to dryness. 1 N HCl (aq) was added and the resulting precipitate was collected by filtration. The solid was dried under vacuum to afford Intermediate 13, 3′-amino-5′-cyano-[1,1′-biphenyl]-4-carboxylic acid (17.0 g, 71.4 mmol, 83% yield) as an off-white solid. Data available in Table 2.
Step (iii): A solution of Intermediate 13, 3′-amino-5′-cyano-[1,1′-biphenyl]-4-carboxylic acid (16.0 g, 67.2 mmol), NaN₃ (8.74 g, 134.4 mmol) and NH₄Cl (7.19 g, 134.4 mmol) in DMF (80 mL) was heated to 130° C. for 16 hours, after which it was cooled and acidified to pH 3 by the addition of 1 N HCl (aq). The resulting precipitate was collected by filtration and dried under vacuum to afford Intermediate 28, 3′-amino-5′-(1H-tetrazol-5-yl)-[1,1′-biphenyl]-4-carboxylic acid (13.0 g, 46.3 mmol, 69% yield) as an off-white solid. Data available in Table 2.
Step (iv): To a solution of Intermediate 28, 3′-amino-5′-(1H-tetrazol-5-yl)-[1,1′-biphenyl]-4-carboxylic acid (12.5 g, 44.4 mmol) in EtOH (120 mL) were added TEA (12.4 mL, 88.9 mmol) and Intermediate 11, diethyl squarate (7.50 g, 44.4 mmol). The mixture was stirred at RT for 16 hours, after which it was acidified with 1 N HCl (aq). The resulting precipitate was collected by filtration and dried under vacuum to afford Intermediate 29, 3′-((2-ethoxy-3,4-dioxocyclobut-1-en-1-yl)amino)-5′-(1H-tetrazol-5-yl)-[1,1′-biphenyl]-4-carboxylic acid (15.0 g, 37.0 mmol, 83% yield) as brown solid. Data available in Table 2.

### Route 10

### Procedure for the Preparation of Intermediate 31, 3-((3-bromo-5-(1H-tetrazol-5-yl)phenyl)amino)-4-ethoxycyclobut-3-ene-1,2-dione

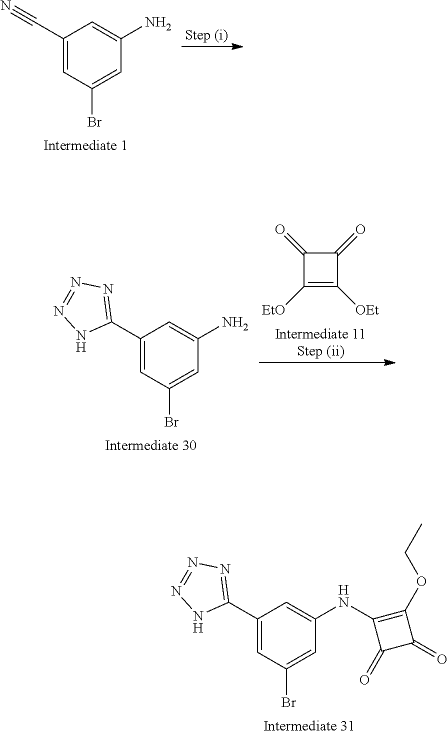 [0229] Step (i): To a suspension of Intermediate 1, 3-amino-5-bromobenzonitrile (26.0 g, 132 mmol) in 2-propanol (500 mL) were added NaN₃ (43.1 g, 663.3 mmol) and ZnCl₂ (90.4 g, 663.3 mmol). The mixture was stirred at 130° C. for 4 hours, after which it was filtered through Celite. The filtrate was concentrated, and the residue was partitioned between EtOAc and sat. aq. NaHCO₃. The Organic layer was separated, the aqueous layer was further extracted with EtOAc (×3) and the combined organics were dried over Na₂SO₄ and concentrated to afford Intermediate 30, 3-bromo-5-(1H-tetrazol-5-yl)aniline (26.8 g, 112.2 mmol, 85% yield) as yellow solid which was used without further purification. Data available in Table 2.
Step (ii): To a solution of Intermediate 30, 3-bromo-5-(1H-tetrazol-5-yl)aniline (10.0 g, 27.5 mmol) in DCM (100 mL) was added TEA (11.7 mL, 83.6 mmol). The mixture was stirred for 10 minutes, after which Intermediate 11, diethyl squarate (6.18 mL, 41.8 mmol) was added. The mixture was stirred at RT for 4 hours after which it was concentrated under reduced pressure. The crude product was purified by flash column chromatography (normal phase, silica) under a gradient of MeOH (0% to 15%) in DCM to afford Intermediate 31, 3-((3-bromo-5-(1H-tetrazol-5-yl)phenyl)amino)-4-ethoxycyclobut-3-ene-1,2-dione (3.80 g, 10.4 mmol, 38% yield) as an off-white solid. Data available in Table 2.

### Route 11

### Procedure for the Preparation of Intermediate 35, (5-(4,4,5,5-tetramethyl-1,3,2-dioxaborolan-2-yl)-2-((4-(trifluoromethyl)benzyl)oxy)pyridine)

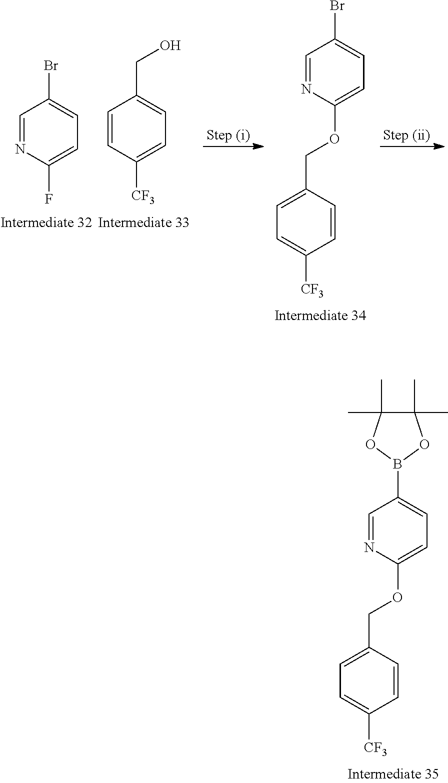 [0232] Step (i): To a solution of Intermediate 33, [4-(trifluoromethyl)phenyl]methanol (3.00 g, 17.0 mmol) in DMF (30 mL) was added K₂CO₃ (3.53 g, 20.2 mmol) at RT. The mixture was stirred for 10 minutes after which Intermediate 32, 5-bromo-2-fluoropyridine (2.99 g, 17.0 mmol) was added. The mixture was heated to 80° C. for 2 hours, after which it was cooled and partitioned between EtOAc and water. The aqueous layer was further extracted with EtOAc and the combined organics were dried over Na₂SO₄ and concentrated. The crude product was purified by flash column chromatography (normal phase, silica) under a gradient of EtOAc (0% to 5%) in hexane to afford Intermediate 34, (5-bromo-2-((4-(trifluoromethyl)benzyl)oxy)pyridine) (2.00 g, 6.02 mmol, 35% yield) as an off-white solid. Data available in Table 2.
Step (ii): A solution of Intermediate 34, (5-bromo-2-((4-(trifluoromethyl)benzyl)oxy)pyridine (1.20 g, 3.61 mmol), KOAc (1.06 g, 10.9 mmol), bis(pinacolato)diboron (2.76 g, 10.9 mmol) and PdCl₂(dppf) (0.13 g, 0.18 mmol) in 1,4-dioxane (20 mL) under nitrogen was heated to 80° C. for 4 hours after which it was cooled and partitioned between EtOAc and water. The aqueous layer was further extracted with EtOAc and the combined organics were dried over Na₂SO₄ and concentrated. The crude product was purified by flash column chromatography (normal phase, silica) under a gradient of EtOAc (0% to 8%) in hexane to afford Intermediate 35, (5-(4,4,5,5-tetramethyl-1,3,2-dioxaborolan-2-yl)-2-((4-(trifluoromethyl)benzyl)oxy)pyridine) (1.20 g, 3.17 mmol, 87%) as white semi-solid. Data available in Table 2.

### Route 12

### Procedure for the Preparation of Intermediate 40, 2-benzyl-5-(4,4,5,5-tetramethyl-1,3,2-dioxaborolan-2-yl)-2H-indazole

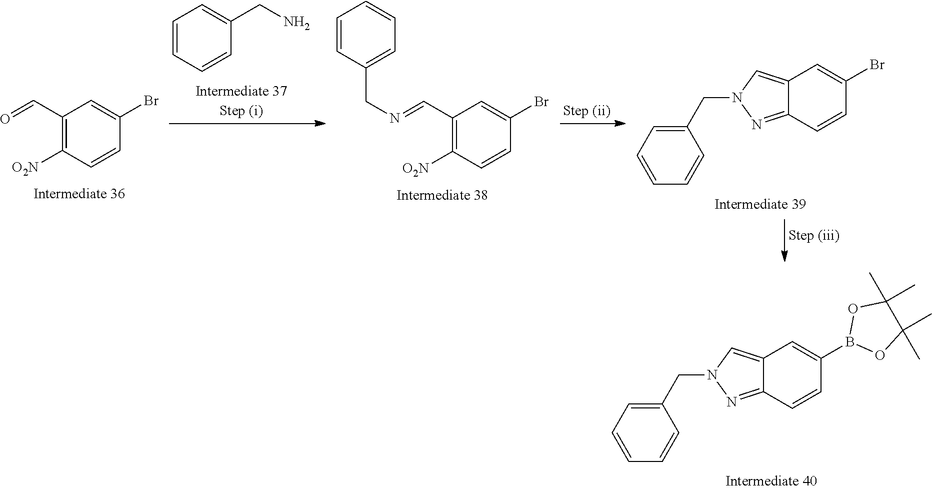 [SMILES: O=Cc1cc(Br)ccc1[N+](=O)[O-] ; NCc1ccccc1 ; O=[N+]([O-])c1ccc(Br)cc1/C=N/Cc1ccccc1 ; Brc1ccc2nn(Cc3ccccc3)cc2c1 ; CC1(C)OB(c2ccc3nn(Cc4ccccc4)cc3c2)OC1(C)C] [0235] Step (i): A mixture of Intermediate 36, 5-bromo-2-nitrobenzaldehyde (3.00 g, 13.1 mmol) and Intermediate 37, benzylamine (1.82 g, 17.0 mmol) in toluene (30 mL) was heated to 80° C. for 4 hours. The reaction was cooled to room temperature and concentrated to give crude Intermediate 38, N-benzyl-1-(5-bromo-2-nitrophenyl)methanimine (2.10 g) as a dark brown liquid. Data available in Table 2.
Step (ii): Intermediate 38, N-benzyl-1-(5-bromo-2-nitrophenyl)methanimine (2.10 g, 6.60 mmol) was dissolved in triethyl phosphite (3.40 mL, 19.8 mmol) and the mixture was heated to 210° C. for 5 minutes under microwave irradiation. The mixture was partitioned between EtOAc and water, then separated. The aqueous layer was extracted with EtOAc (×2) and the combined organics were dried over Na₂SO₄, then concentrated. The crude material was purified by flash column chromatography (normal phase, silica) under a gradient of EtOAc (0% to 20%) in hexane to afford Intermediate 39, 2-benzyl-5-bromo-2H-indazole (1.30 g, 4.54 mmol, 35% yield over 2 steps) as a brown gum. Data available in Table 2.
Step (iii): Intermediate 39, 2-benzyl-5-bromo-2H-indazole (1.30 g, 4.54 mmol), KOAc (1.34 g, 13.6 mmol), bis(pinacolato)diboron (3.46 g, 13.6 mmol) and PdCl₂(dppf) (0.17 g, 0.22 mmol) were dissolved in 1,4-dioxane (15 mL) under a nitrogen atmosphere. The reaction mixture was stirred at 80° C. for 4 hours, after which it was cooled to room temperature and partitioned between EtOAc and water. The organics were separated, and the aqueous layer was further extracted with EtOAc. The combined organics were dried over Na₂SO₄ and concentrated to afford Intermediate 40, 2-benzyl-5-(4,4,5,5-tetramethyl-1,3,2-dioxaborolan-2-yl)-2H-indazole (1.0 g, 2.99 mmol, 66% yield) as a brown gum that was used without further purification. Data available in Table 2.

### Route 13

### Procedure for the Preparation of Intermediate 44, 2-(4-(trifluoromethyl)phenoxy)ethan-1-amine, hydrochloride salt

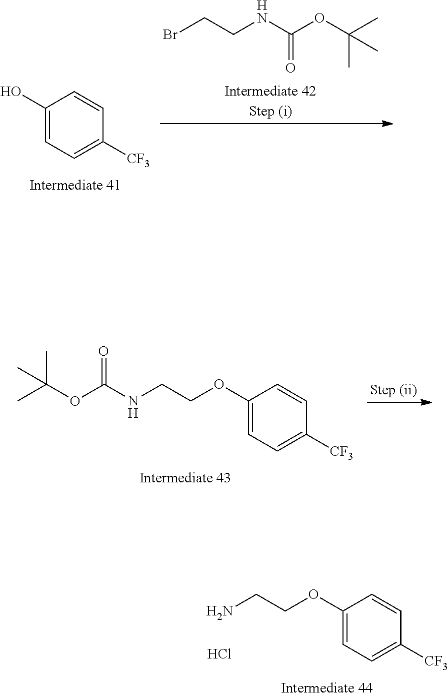 [SMILES: CC(C)(C)OC(=O)NCCBr ; Oc1ccc(C(F)(F)F)cc1 ; CC(C)(C)OC(=O)NCCOc1ccc(C(F)(F)F)cc1 ; Cl ; NCCOc1ccc(C(F)(F)F)cc1] [0239] Step (i): To a solution of Intermediate 42, tert-butyl (2-bromoethyl)carbamate (876 mg, 3.9 mmol) in DMF (10 mL) were added Intermediate 41, 4-(trifluoromethyl)phenol (422 mg, 2.6 mmol) and Cs₂CO₃ (1.25 g, 3.8 mmol). The mixture was heated to 90° C. for 4 hours, after which it was cooled and poured into water. The mixture was extracted with EtOAc, and the organics were washed with brine, dried via passage through a hydrophobic frit and concentrated. The crude material was purified by flash column chromatography (normal phase, silica) under a gradient of EtOAc (0% to 50%) in pet. ether to afford Intermediate 43, tert-butyl (2-(4-(trifluoromethyl)phenoxy)ethyl)carbamate (793 mg, 2.6 mmol, 100% yield) as a yellow oil. Data available in Table 2.
Step (ii): To a solution of Intermediate 43, tert-butyl (2-(4-(trifluoromethyl)phenoxy)ethyl)carbamate (793 mg, 2.6 mmol) in 1,4-dioxane (2 mL) was added a solution of 4 M HCl in 1,4-dioxane (2 mL). The mixture was stirred for 18 hours, after which MeOH (1 mL) was added. The mixture was stirred for a further 30 minutes, and then concentrated to afford Intermediate 44, 2-(4-(trifluoromethyl)phenoxy)ethan-1-amine, hydrochloride salt (596 mg, 2.5 mmol, 95% yield) as a pale yellow solid. Data available in Table 2.

### Route 14

### Procedure for the Preparation of Intermediate 48, 2-(2-(3,5-bis(trifluoromethyl)phenoxy)ethoxy)-5-bromopyridine

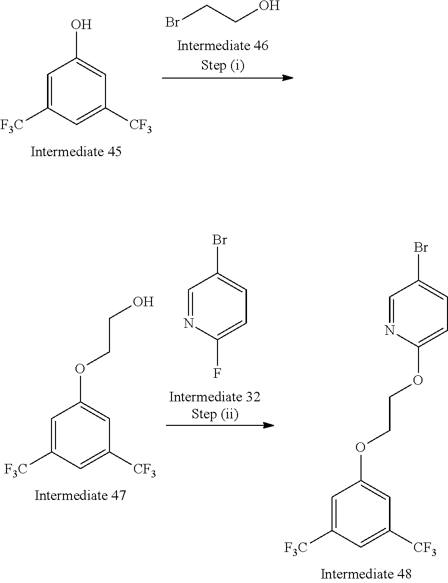 [0242] Step (i): To a solution of Intermediate 45, 3,5-bis(trifluoromethyl)phenol (5.00 g, 21.7 mmol) in DMF (50 mL) was added K₂CO₃ (8.99 g, 65.2 mmol). The mixture was stirred at RT for 20 minutes, after which Intermediate 46, 2-bromoethan-1-ol (9.25 mL, 130 mmol) was added. The mixture was heated to 80° C. for 4 hours, after which it was cooled and partitioned between EtOAc and water. The aqueous layer was further extracted with EtOAc and the combined organics were dried over Na₂SO₄ and concentrated. The crude product was purified by flash column chromatography (normal phase, silica) under a gradient of EtOAc (0% to 20%) in hexane to afford Intermediate 47, 2-(3, 5-bis (trifluoromethyl)phenoxy)ethan-1-01 (3.90 g, 14.2 mmol, 66% yield) as an oil. Data available in Table 2.
Step (ii): To a suspension of NaH (60% in mineral oil) (0.77 g, 19.2 mmol) in DMF (25 mL) at 0° C. was added Intermediate 47, 2-(3,5-bis(trifluoromethyl) phenoxy)ethan-1-ol (3.00 g, 11.0 mmol). The mixture was warmed to RT over 15 minutes, after which Intermediate 32, 5-bromo-2-fluoropyridine (2.30 g, 13.1 mmol) was added. The reaction was stirred at RT for 1 hour, after which it was partitioned between EtOAc and water. The aqueous layer was further extracted with EtOAc and the combined organics were dried over Na₂SO₄ and concentrated. The crude product was purified by flash column chromatography (normal phase, silica) under a gradient of EtOAc (0% to 12%) in hexane to afford Intermediate 48, 2-(2-(3,5-bis(trifluoromethyl)phenoxy)ethoxy)-5-bromopyridine (3.60 g, 8.37 mmol, 77% yield) as yellow semi-solid. Data available in Table 2.

### Route 15

### Procedure for the Preparation of Intermediate 51, 2-(4-(trifluoromethyl)phenoxy)ethan-1-ol

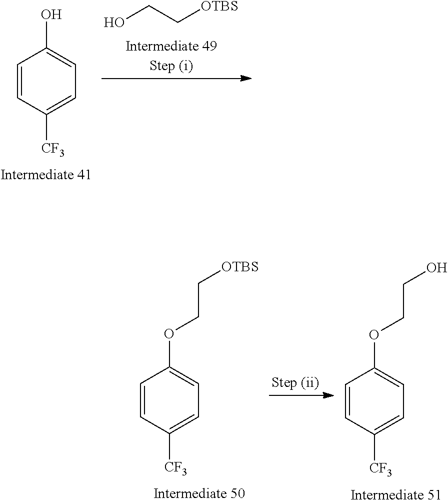 [SMILES: Oc1ccc(C(F)(F)F)cc1 ; CC(C)(C)[Si](C)(C)OCCO ; CC(C)(C)[Si](C)(C)OCCOc1ccc(C(F)(F)F)cc1 ; OCCOc1ccc(C(F)(F)F)cc1] [0245] Step (i): To a solution of Intermediate 41, 4-(trifluoromethyl) phenol (2.80 g, 17.3 mmol), Intermediate 49, 2-((tert-butyldimethylsilyl)oxy)ethan-1-ol (4.00 g, 22.5 mmol) and triphenyl phosphine (5.89 g, 22.5 mmol) in toluene (30 mL) at 0° C. was added DIAD (4.40 mL, 22.5 mmol) dropwise. The mixture was stirred at RT for 16 hours, after which it was partitioned between EtOAc and water. The aqueous layer was further extracted with EtOAc and the combined organics were dried over Na₂SO₄ and concentrated. The crude material was purified by flash column chromatography (normal phase, silica) under a gradient of EtOAc (0% to 2%) in hexane to afford Intermediate 50, tert-butyldimethyl(2-(4-(trifluoromethyl)phenoxy)ethoxy)silane (3.40 g, 10.6 mmol, 61% yield) as a yellow liquid. Data available in Table 2.
Step (ii): To a solution of Intermediate 50, tert-butyldimethyl(2-(4-(trifluoromethyl) phenoxy)ethoxy)silane (3.40 g, 10.6 mmol) in THF (30 mL) at 0° C. was added 1 N HCl (aq) (30 mL). The mixture was stirred at RT for 1 hour, after which it was partitioned between sat. aq. NaHCO₃ and EtOAc. The organics were separated, dried over Na₂SO₄ and concentrated. The crude material was purified by flash column chromatography (normal phase, silica) under a gradient of EtOAc (0% to 30%) in hexane to afford Intermediate 51, 2-(4-(trifluoromethyl)phenoxy)ethan-1-ol (2.50g, 12.1 mmol, assumed quantitative) as a yellow liquid. Data available in Table 2.

### Route 16

### Procedure for the Preparation of Intermediate 54, (4-((4-carbamoylbenzyl)carbamoyl)phenyl)boronic acid

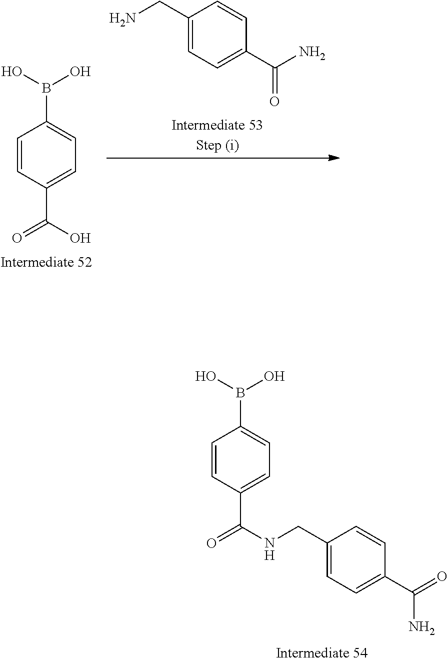 [SMILES: O=C(O)c1ccc(B(O)O)cc1 ; NCc1ccc(C(N)=O)cc1 ; NC(=O)c1ccc(CNC(=O)c2ccc(B(O)O)cc2)cc1] [0248] Step (i): To a solution of Intermediate 52, 4-boronobenzoic acid (795 mg, 4.79 mmol) in DMF (5 mL) under ice cooling was added HATU (2.27 g, 5.99 mmol). The mixture was stirred at the same temperature for 10 minutes after which Intermediate 53, 4-(aminomethyl) benzamide (600 mg, 3.99 mmol) and DIPEA (2.02 mL, 11.98 mmol) were added sequentially. The mixture was warmed to RT and stirred for 6 hours, after which it was partitioned between EtOAc and water. The organics were separated, and the aqueous layer was washed with EtOAc. The combined organics were dried over Na₂SO₄ and concentrated to afford Intermediate 54, (4-((4-carbamoylbenzyl)carbamoyl)phenyl)boronic acid (0.350 g, 1.17 mmol, 29% yield) as yellow solid. Data available in Table 2.

### Route 17

### Procedure for the Preparation of Intermediate 60, N-(4-methoxybenzyl)-6-phenoxy-4-(1H-tetrazol-5-yl)pyridin-2-amine

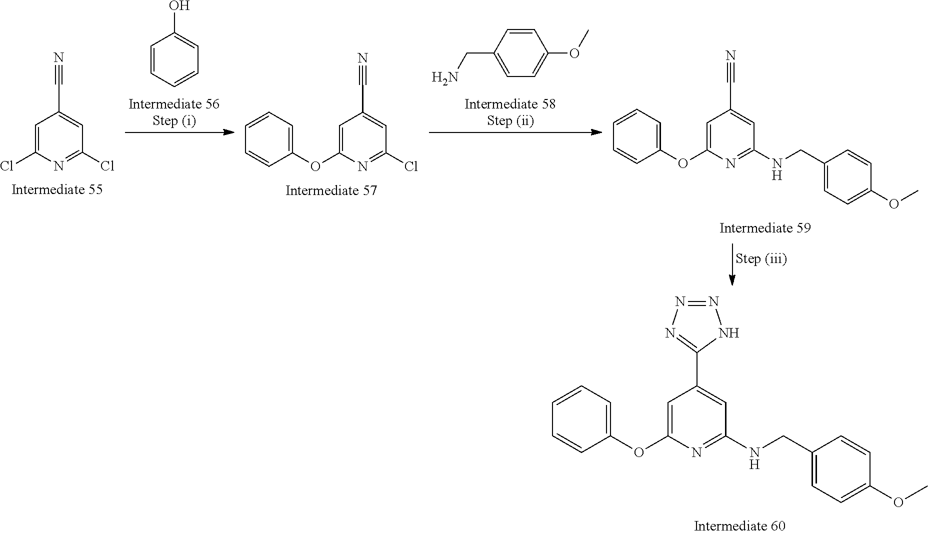 [0250] Step (i): To a solution of Intermediate 56, phenol (1.31 g, 14.0 mmol) in DMSO (40 mL) was added K₂CO₃ (2.4 g, 17.4 mmol). The mixture was stirred at RT for 30 minutes, after which Intermediate 55, 2,6-dichloroisonicotinonitrile (2.0 g, 11.6 mmol) was added. The mixture was stirred at RT for 24 hours, after which it was poured into water and extracted with EtOAc (×3). The combined organics were washed with brine, dried over Na₂SO₄ and concentrated. The crude material was purified by flash column chromatography (normal phase, silica) under a gradient of EtOAc (0% to 20%) in pet. ether to afford Intermediate 57, 2-chloro-6-phenoxyisonicotinonitrile (2.5 g, 10.9 mmol, 94% yield) as a yellow liquid. Data available in Table 2.
Step (ii): A solution of Intermediate 57, 2-chloro-6-phenoxyisonicotinonitrile (2.5 g, 10.8 mmol) and Intermediate 58, 4-methoxybenzylamine (1.63 g, 11.9 mmol) in NMP (26 mL) was heated to 120° C. for 3 hours, after which the mixture was cooled and poured into water. The precipitate was filtered, and the crude material was purified by flash column chromatography (normal phase, silica) under a gradient of EtOAc (10% to 50%) in pet. ether to afford Intermediate 59, 2-((4-methoxybenzyl)amino)-6-phenoxyisonicotinonitrile (1.7 g, 5.1 mmol, 47% yield) as an off white solid. Data available in Table 2.
Step (iii): A solution of Intermediate 59, 2-((4-methoxybenzyl)amino)-6-phenoxyisonicotinonitrile (1.7 g, 5.13 mmol), NaN₃ (1.33 g, 20.5 mmol) and NH₄Cl (1.1 g, 20.5 mmol) in DMF (9.2 mL) was heated to 130° C. for 2 hours. The mixture was cooled and poured into water. The aqueous mixture was acidified to pH 1 using 1 N HCl (aq) and stirred for 30 minutes, after which it was extracted with EtOAc (×3). The combined organics were washed with brine, dried over Na₂SO₄ and concentrated. The crude material was purified by flash column chromatography (normal phase, silica) under a gradient of MeOH (0% to 5%) in DCM to afford Intermediate 60, N-(4-methoxybenzyl)-6-phenoxy-4-(1H-tetrazol-5-yl)pyridin-2-amine (1.6 g, 4.27 mmol, 83% yield) as a brown gum. Data available in Table 2.

### Route 18

### Procedure for the Preparation of Intermediate 61, 6-phenoxy-4-(1H-tetrazol-5-yl)pyridin-2-amine

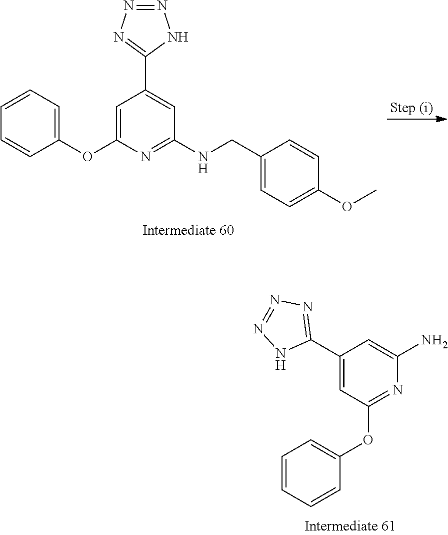 [SMILES: COc1ccc(CNc2cc(-c3nnn[nH]3)cc(Oc3ccccc3)n2)cc1 ; "Step (i)" ; Nc1cc(-c2nnn[nH]2)cc(Oc2ccccc2)n1] [0254] Step (iv): A solution of Intermediate 60, N-(4-methoxybenzyl)-6-phenoxy-4-(1H-tetrazol-5-yl) pyridin-2-amine (1.6 g, 4.27 mmol) in TFA (16 mL) was heated to 70° C. for 24 hours in a sealed tube, after which the mixture was cooled and concentrated to dryness. Trituration of the crude material with DCM afforded Intermediate 61, 6-methoxy-4-(1H-tetrazol-5-yl)pyridin-2-amine (0.75 g, 3.91 mmol, 93% yield) as a brown solid. Data available in Table 2.

### Route 19

### Procedure for the Preparation of Intermediate 63, 6-chloro-N-(4-methoxybenzyl)-4-(1H-tetrazol-5-yl)pyridin-2-amine

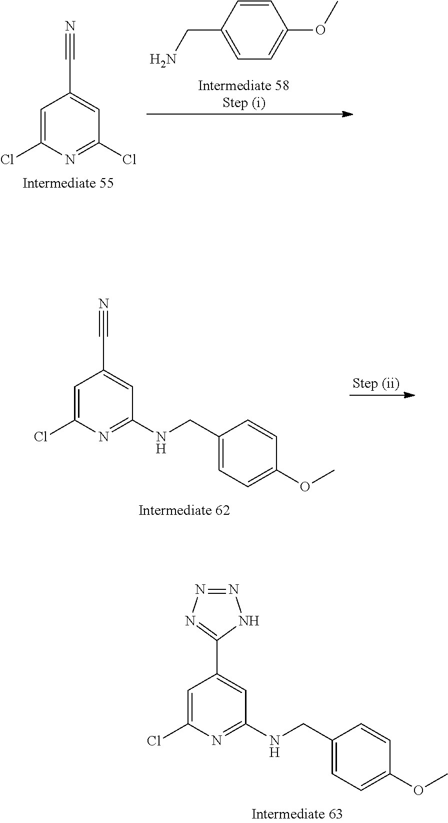 [0256] Step (i): A solution of Intermediate 55, 2,6-dichloroisonicotinonitrile (4.5 g, 26.2 mmol), and Intermediate 58, 4-methoxybenzylamine (3.6 g, 26.2 mmol) in NMP (45 mL) was heated to 110° C. for 3 hours. The mixture was cooled and poured into ice-cold water (500 mL). The resulting precipitate was filtered, washed with water and dried under vacuum to afford Intermediate 62, 2-chloro-6-((4-methoxybenzyl)amino)isonicotinonitrile (3.6 g, 13.2 mmol, 50% yield) as an off-white solid. Data available in Table 2.
Step (ii): A solution of Intermediate 62, 2-chloro-6-((4-methoxybenzyl)amino) isonicotinonitrile (1.36 g, 5 mmol), NaN₃ (650 mg, 10 mmol) and NH₄Cl (535 mg, 10 mmol) in DMF (9 mL) was heated to 130° C. for 2 hours. The mixture was cooled and poured into water. The aqueous mixture was acidified to pH 1 using 1 N HCl (aq) and the resulting precipitate was filtered, washed with water and dried under vacuum to afford Intermediate 63, 6-chloro-N-(4-methoxybenzyl)-4-(1H-tetrazol-5-yl)pyridin-2-amine (960 mg, 3.04 mmol, 61% yield) as an off-white solid. Data available in Table 2.

### Route 20

### Procedure for the Preparation of Intermediate 64, 6-methoxy-N-(4-methoxybenzyl)-4-(1H-tetrazol-5-yl)pyridin-2-amine

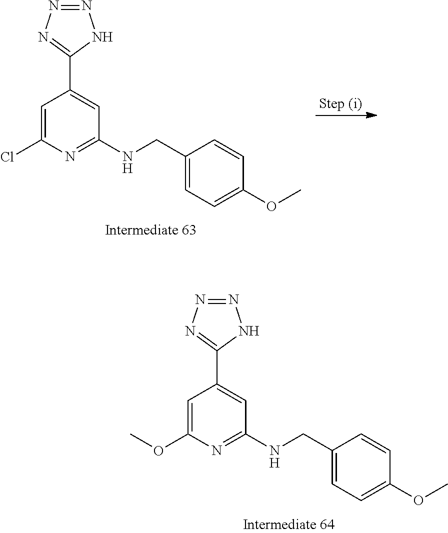 [0259] Step (i): To a solution of Intermediate 63, 6-chloro-N-(4-methoxybenzyl)-4-(1H-tetrazol-5-yl)pyridin-2-amine (1.0 g, 3.16 mmol) in DMSO (6.3 mL) was added NaOMe (30 wt. % in MeOH, 1.14 mL, 6.33 mmol). The mixture was heated to 120° C. for 20 hours, after which it was cooled and poured into water. The aqueous mixture was acidified to pH 1 with 1 N HCl (aq) and the resulting precipitate was collected. The crude material was purified by flash column chromatography (normal phase, silica) under a gradient of MeOH (0% to 10%) in chloroform to afford Intermediate 64, 6-methoxy-N-(4-methoxybenzyl)-4-(1H-tetrazol-5-yl)pyridin-2-amine (700 mg, 2.24 mmol, 71% yield) as an off-white solid. Data available in Table 2.

### Route 21

### Procedure for the Preparation of Intermediate 65, N-(4-methoxybenzyl)-6-phenyl-4-(1H-tetrazol-5-yl)pyridin-2-amine

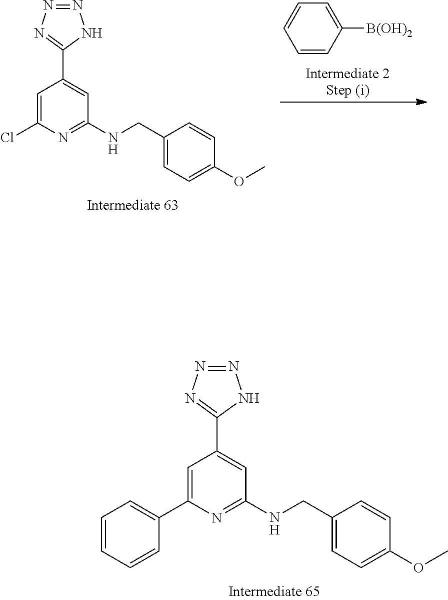 [0261] Step (i): A solution of Intermediate 63, 6-chloro-N-(4-methoxybenzyl)-4-(1H-tetrazol-5-yl)pyridin-2-amine (1 g, 3.15 mmol), Intermediate 2, phenylboronic acid (1.15 g, 9.46 mmol), K₂CO₃ (1.3 g, 9.46 mmol) and Pd(dppf)Cl₂·DCM (257 mg, 0.32 mmol) in 1,4-dioxane (10 mL) was heated to 120° C. for 6 hours, after which the mixture was cooled and poured into water. The aqueous mixture was acidified to pH 1 with 1 N HCl (aq) and the resulting precipitate was collected. The crude material was purified by flash column chromatography (normal phase, silica) under a gradient of MeOH (0% to 8%) in chloroform to afford Intermediate 65, N-(4-methoxybenzyl)-6-phenyl-4-(1H-tetrazol-5-yl)pyridin-2-amine (810 mg, 2.26 mmol, 72% yield) as an off-white solid. Data available in Table 2.

### Route 22

### Procedure for the Preparation of Intermediate 71, 3-ethoxy-4-[3-ethyl-5-(1H-tetrazol-5-yl)aniIino]cyclobut-3-ene-1,2-dione

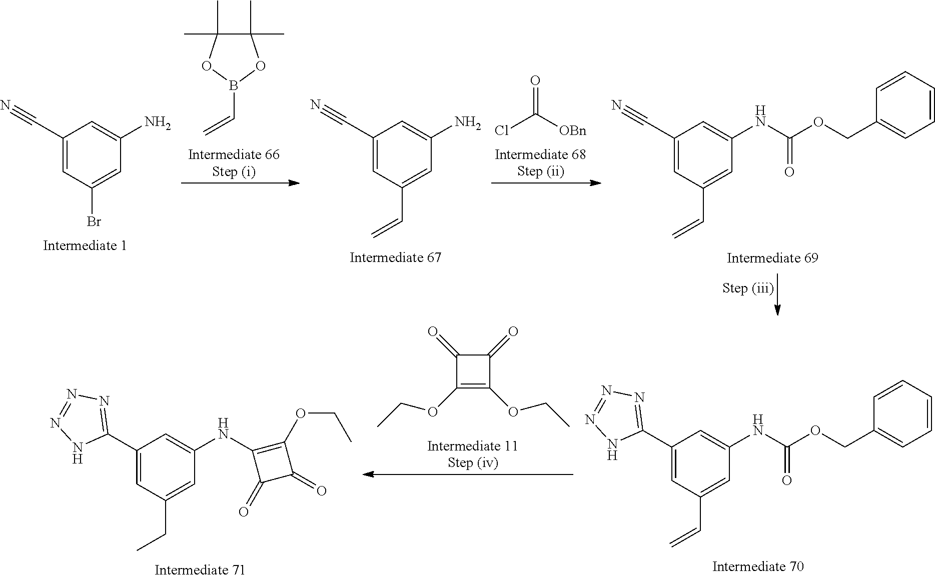 [0263] Step (i): A solution of Intermediate 1, 3-amino-5-bromobenzonitrile (1.0 g, 5.08 mmol), Intermediate 66, 4,4,5,5-tetramethyl-2-vinyl-1,3,2-dioxaborolane (1.29 mL, 7.61 mmol), K₂CO₃ (1.40 g, 10.2 mmol), and Pd(Ph₃P)₄ (0.118 g, 0.10 mmol) in 1,4-dioxane (24 mL) and water (6 mL) was heated to 80° C. for 18 hours, after which it was cooled to RT and partitioned between EtOAc and water. The organics were separated, washed with brine, dried via passage through a hydrophobic frit and concentrated. The crude material was purified by flash column chromatography (normal phase, silica) under a gradient of EtOAc (0% to 25%) in isohexane to afford Intermediate 67, 3-amino-5-vinylbenzonitrile (695 mg, 4.82 mmol, 95% yield) as an orange solid. Data available in Table 2.
Step (ii): To solution of Intermediate 67, 3-amino-5-vinylbenzonitrile (640 mg, 4.44 mmol), and K₂CO₃ (927 mg, 6.7 mmol) in THF (37 mL) was added intermediate 68, benzyl chloroformate (0.95 mL, 6.7 mmol). The mixture was stirred at RT for 18 hours, after which it was partitioned between EtOAc and water. The organics were separated, washed with brine, dried via passage through a hydrophobic frit and concentrated. The crude material was purified by flash column chromatography (normal phase, silica) under a gradient of Et₂O (0% to 60%) in isohexane to afford Intermediate 69, benzyl (3-cyano-5-vinylphenyl)carbamate (944 mg, 3.40 mmol, 76% yield) as an off-white solid. Data available in Table 2.
Step (iii): A solution of Intermediate 69, benzyl (3-cyano-5-vinylphenyl)carbamate (614 mg, 2.2 mmol), NaN₃ (287 mg, 4.4 mmol), and NH₄Cl (235 mg, 4.4 mmol) in DMF (6 mL) was heated to 130° C. for 2 hours. After this time, the mixture was cooled and partitioned between 1 M HCl (aq) and EtOAc. The organics were separated, washed with brine, dried via passage through a hydrophobic frit and concentrated. The crude material was purified by flash column chromatography (reversed phase, C18) under a gradient of MeCN (10% to 60%) in water (0.1% v/v HCOOH) to afford Intermediate 70, benzyl (3-(1H-tetrazol-5-yl)-5-vinylphenyl)carbamate (355 mg, 1.12 mmol, 50% yield) as a beige solid. Data available in Table 2.
Step (iv): A mixture of Intermediate 70, benzyl (3-(1H-tetrazol-5-yl)-5-vinylphenyl)carbamate (270 mg, 0.84 mmol), TEA (0.12 mL, 0.84 mmol) and 10% Pd/C (60 mg) in MeOH (4 mL) was stirred under an atmosphere of hydrogen gas for 3.5 hours. The mixture was filtered through Celite (washing through with EtOAc) and concentrated. The reside was dissolved in EtOH (4 mL) and Intermediate 11, diethyl squarate (0.12 mL, 0.84 mmol) was added. This mixture was stirred at RT for 3 hours, after which it was concentrated. The crude material was purified by flash column chromatography (reversed phase, C18) under a gradient of MeCN (10% to 80%) in water (0.1% v/v HCOOH) to afford Intermediate 71, 3-ethoxy-4-[3-ethyl-5-(1H-tetrazol-5-yl)anilino]cyclobut-3-ene-1,2-dione (203 mg, 0.65 mmol, 77% yield) as a cream solid. Data available in Table 2.

### General Synthetic Procedures for the Examples

### Route A

### Procedure for the Preparation of Example 2, 3-((3-(1H-tetrazol-5-yl)-5-(trifluoromethyl)phenyl)amino)-4-hydroxycyclobut-3-ene-1,2-dione

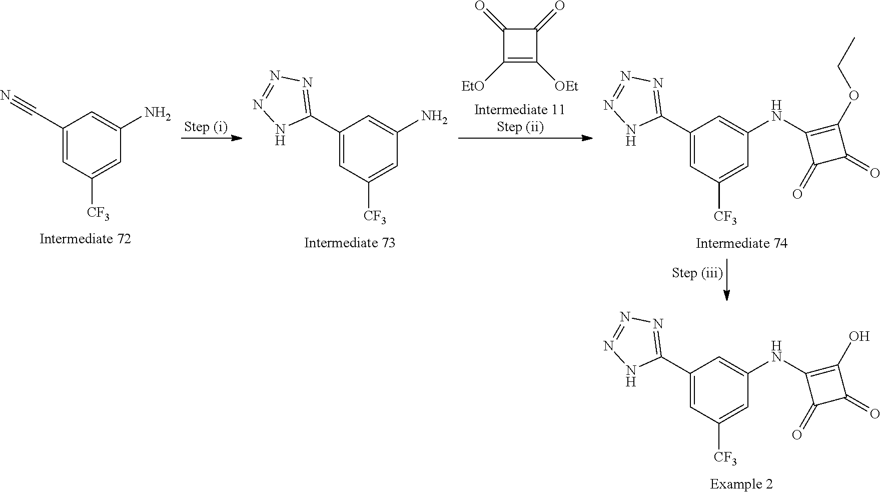 [0268] Step (i): To a solution of Intermediate 72, 3-amino-5-(trifluoromethyl)benzonitrile (150 mg, 0.81 mmol) in DMF (3 mL) were added NaN₃ (65 mg, 1.62 mmol) and NH₄Cl (53.5 mg, 1.62 mmol). The mixture was heated to 130° C. for 20 hours, after which it was cooled to RT and acidified to pH 1 with 1 M HCl (aq). The mixture was extracted with EtOAc and the organics were washed with water and brine, dried via passage through a hydrophobic frit and concentrated. The crude material was purified by reversed phase flash chromatography (C18 silica) under a gradient of MeCN (0-50%) in modified water (containing 0.1% v/v HCOOH) to afford the product Intermediate 73, 3-(1H-tetrazol-5-yl)-5-(trifluoromethyl)aniline (144 mg, 0.63 mmol, 77% yield). Data available in Table 2.
Step (ii): To a solution of Intermediate 73, 3-(1H-tetrazol-5-yl)-5-(trifluoromethyl)aniline (77 mg, 0.34 mmol) in EtOH (1.4 mL) were added Intermediate 11, diethyl squarate (0.05 mL, 0.34 mmol) and TEA (0.05 mL, 0.38 mmol). The mixture was stirred at RT for 1 hour, after which it was concentrated to dryness. The crude material was purified by reversed phase flash chromatography (C18 silica) under a gradient of MeCN (0-60%) in modified water (0.1% v/v HCOOH) to afford the product Intermediate 74, 3-((3-(1H-tetrazol-5-yl)-5-(trifluoromethyl)phenyl)amino)-4-ethoxycyclobut-3-ene-1,2-dione (51.6 mg, 0.15 mmol, 44% yield). Data available in Table 2.
Step (iii): Intermediate 74, 3-((3-(1H-tetrazol-5-yl)-5-(trifluoromethyl)phenyl)amino)-4-ethoxycyclobut-3-ene-1,2-dione (39 mg, 0.11 mmol) was dissolved in a 10:1 mixture of THF and 1 M HCl (aq) (0.55 mL) and heated to 60° C. for 3 hours, after which it was concentrated. The crude material was purified by prep HPLC [reversed phase (Kinetex C18, 100×30 mm, 5 μm, 30 mL per min, gradient of Solvent B in Solvent A: 5% to 95% Solvent B (over 10 min), 100% Solvent B (for 2 min); Solvent A: 0.1% TFA in water. Solvent B: MeCN] to afford Example 2, 3-((3-(1H-tetrazol-5-yl)-5-(trifluoromethyl)phenyl)amino)-4-hydroxycyclobut-3-ene-1,2-dione (15.3 mg, 0.047 mmol, 43% yield). Data available in Table 3.

### Route B

### Procedure for the Preparation of Example 5, N-benzyl-3′-((2-hydroxy-3,4-dioxocyclobut-1-en-1-yl)amino)-5′-(1H-tetrazol-5-yl)-[1,1′-biphenyl]-4-carboxamide

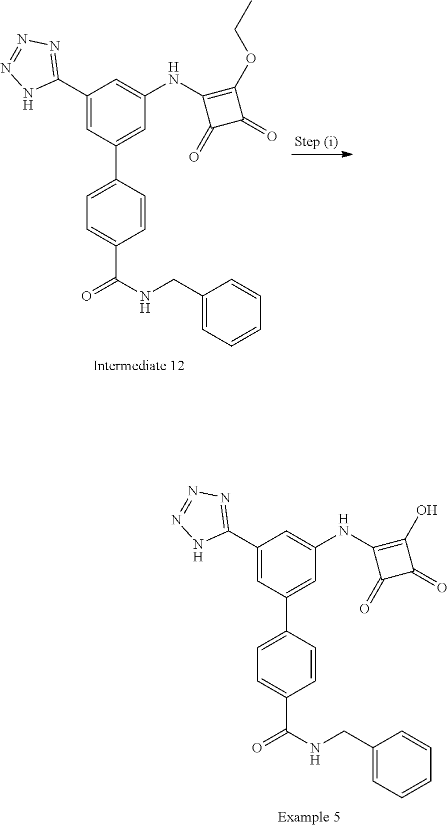 [SMILES: CCOc1c(Nc2cc(-c3ccc(C(=O)NCc4ccccc4)cc3)cc(-c3nnn[nH]3)c2)c(=O)c1=O ; O=C(NCc1ccccc1)c1ccc(-c2cc(Nc3c(O)c(=O)c3=O)cc(-c3nnn[nH]3)c2)cc1] [0272] Step (i): A suspension of Intermediate 12, N-benzyl-3′-((2-ethoxy-3,4-dioxocyclobut-1-en-1-yl)amino)-5′-(1H-tetrazol-5-yl)-[1,1′-biphenyl]-4-carboxamide (1.43 g, 2.89 mmol) in THF (15 mL) and 1 M HCl (aq) (1.5 mL) was heated to 60° C. for 24 hours, after which time the mixture was concentrated. The crude material was purified by prep HPLC [reversed phase (Gemini-NX C18, 100×30 mm, 5 μm, 30 mL per min, gradient of Solvent B in Solvent A: 5% to 35% Solvent B (over 10 min), 100% Solvent B (for 2 min); Solvent A: water containing 0.2% v/v 28% ammonia solution. Solvent B: MeCN]. The resulting solid was suspended in 1 M HCl (aq) and stirred vigorously for 2 hours, after which it was collected by filtration. The wet solid was suspended in toluene and concentrated to afford Example 5, N-benzyl-3′-((2-hydroxy-3,4-dioxocyclobut-1-en-1-yl)amino)-5′-(1H-tetrazol-5-yl)-[1,1′-biphenyl]-4-carboxamide (720 mg, 1.55 mmol, 53% yield) as a light green solid. Data available in Table 3.

### Route C

### Procedure for the Preparation of Example 8, 3′-((2-hydroxy-3,4-dioxocyclobut-1-en-1-yl)amino)-N-(4-(2-methoxyethoxy)benzyl)-5′-(1H-tetrazol-5-yl)-[1,1′-biphenyl]-4-carboxamide

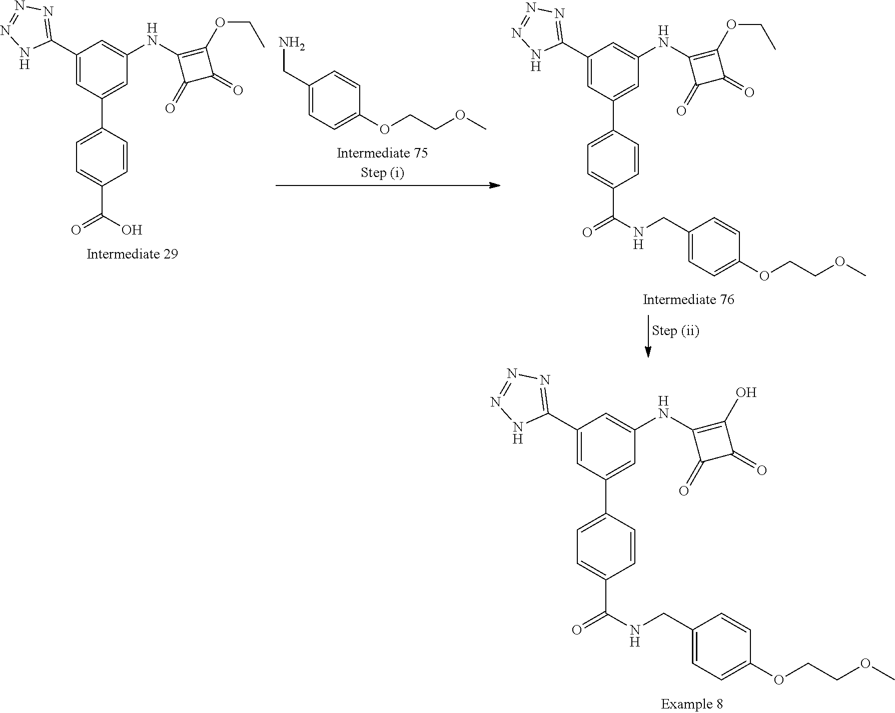 [0274] Step (i): To a solution of Intermediate 29, 3′-((2-ethoxy-3,4-dioxocyclobut-1-en-1-yl)amino)-5′-(1H-tetrazol-5-yl)-[1,1′-biphenyl]-4-carboxylic acid (60 mg, 0.15 mmol) and DIPEA (0.08 mL) in DMF (0.7 mL) was added HATU (68.4 mg, 0.18 mmol), followed by Intermediate 75, (4-(2-methoxyethoxy)phenyl)methanamine (29.5 mg, 0.17 mmol). The mixture was stirred at RT for 1 hour, after which it was diluted with DMSO and purified by prep HPLC [reversed phase (Kinetex C18, 100×30 mm, 5 μm, 30 mL per min, gradient of Solvent B in Solvent A: 40% to 70% Solvent B (over 10 min), 100% Solvent B (for 2 min); Solvent A: water containing 0.1% TFA. Solvent B: MeCN] to afford Intermediate 76, 3′-((2-ethoxy-3,4-dioxocyclobut-1-en-1-yl)amino)-N-(4-(2-methoxyethoxy)benzyl)-5′-(1H-tetrazol-5-yl)-[1,1′-biphenyl]-4-carboxamide (36.3 mg, 0.064 mmol, 43% yield) as a pale green solid. Data available in Table 2.
Step (ii): A suspension of Intermediate 76, 3′-((2-ethoxy-3,4-dioxocyclobut-1-en-1-yl)amino)-N-(4-(2-methoxyethoxy)benzyl)-5′-(1H-tetrazol-5-yl)-[1,1′-biphenyl]-4-carboxamide (36.3 mg, 0.064 mmol) in THF (1 mL) and 1 M HCl (aq) (0.1 mL) was heated to 60° C. for 13 hours, after which it was concentrated to dryness. The crude material was purified by prep HPLC [reversed phase (Kinetex C18, 100×30 mm, 5 μm, 30 mL per min, gradient of Solvent B in Solvent A: 40% to 30% Solvent B (over 10 min), 100% Solvent B (for 2 min); Solvent A: water containing 0.1% TFA. Solvent B: MeCN] to afford Example 8, 3′-((2-hydroxy-3,4-dioxocyclobut-1-en-1-yl)amino)-N-(4-(2-methoxyethoxy)benzyl)-5′-(1H-tetrazol-5-yl)-[1,1′-biphenyl]-4-carboxamide (16 mg, 0.03 mmol, 46% yield) as a pale green solid. Data available in Table 3.

### Route D

### Procedure for the Preparation of Example 15, 3-((5-(1H-tetrazol-5-yl)-4′-(trifluoromethyl)-[1,1′-biphenyl]-3-yl)amino)-4-hydroxycyclobut-3-ene-1,2-dione

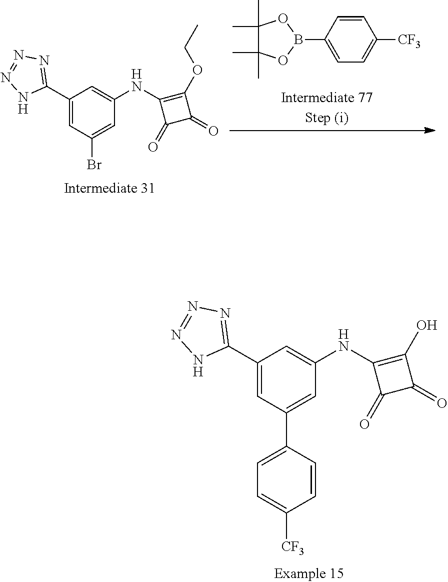 [0277] Step (i): A suspension of Intermediate 31, 3-((3-bromo-5-(1H-tetrazol-5-yl)phenyl)amino)-4-ethoxycyclobut-3-ene-1,2-dione (0.50 g, 1.37 mmol), Intermediate 77, 4,4,5,5-tetramethyl-2-(4-(trifluoromethyl)phenyl)-1,3,2-dioxaborolane (0.56 g, 2.06 mmol) and K₂CO₃ (0.38 g, 2.75 mmol) in MeCN (5 mL) and water (5 mL) was degassed with N₂ for 15 minutes. PdCl₂(dtbpf) (0.089 g, 0.13 mmol) was added and the mixture was heated to 100° C. under microwave irradiation for 1 hour. After this time, the solvents were removed under reduced pressure and the crude material was purified by prep HPLC [reversed phase (X-bridge C18, 250×30 mm, 5 μm, 27 mL per min, gradient of Solvent B in Solvent A: 10% to 98% Solvent B (over 59 min), 10% Solvent B (for 2 min); Solvent A: 10 mM NH₄HCO₃ in water. Solvent B: MeCN] to afford Example 15, 3-((5-(1H-tetrazol-5-yl)-4′-(trifluoromethyl)-[1,1′-biphenyl]-3-yl)amino)-4-hydroxycyclobut-3-ene-1,2-dione (0.35 g, 0.87 mmol, 64% yield) as brown solid. Data available in Table 3.

### Route E

### Procedure for the Preparation of Example 17, 3-hydroxy-4-((6-methoxy-4-(1H-tetrazol-5-yl)pyridin-2-yl)amino)cyclobut-3-ene-1,2-dione

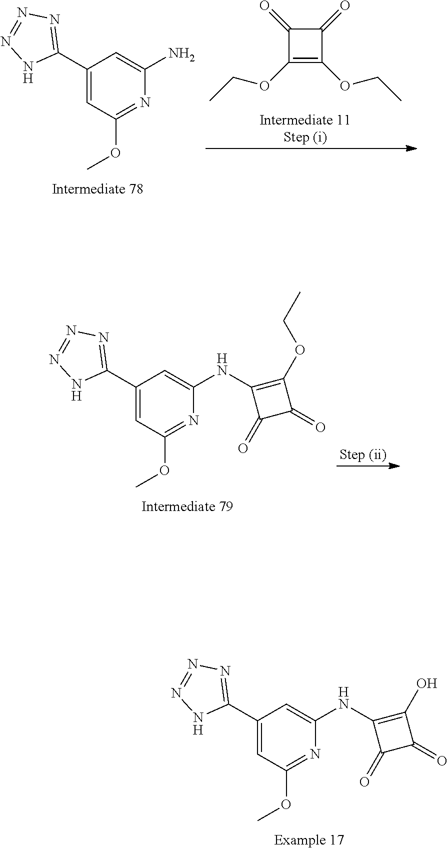 [0279] Step (i): To an ice-cold suspension of Intermediate 78, 6-methoxy-4-(1H-tetrazol-5-yl)pyridin-2-amine (510 mg, 2.56 mmol) in EtOH (10 mL) was added TEA (1.00 mL, 7.69 mmol). The mixture was stirred at the same temperature for 15 mins after which Intermediate 11, diethyl squarate (479 mg, 2.82 mmol) was added drop-wise and the reaction was stirred at RT for 16 hours. The mixture was concentrated, and the residue was partitioned between EtOAc and water. The organics were removed, and the aq. layer was acidified to pH 1-2 using 1 N aq. HCl and stirred for 30 minutes. The resulting precipitate was collected by filtration, which was washed with ice-cold water then dried under high vacuum to afford Intermediate 79, 3-ethoxy-4-((6-methoxy-4-(1H-tetrazol-5-yl)pyridin-2-yl)amino)cyclobut-3-ene-1,2-dione (350 mg, 1.11 mmol, 43% yield) as a yellow solid. Data available in Table 2.
Step (ii): A suspension of Intermediate 79, 3-ethoxy-4-((6-methoxy-4-(1H-tetrazol-5-yl)pyridin-2-yl)amino)cyclobut-3-ene-1,2-dione (350 mg, 1.11 mmol) in THF (5.5 mL) and 1 N aq. HCl (0.55 mL) was heated to 60° C. for 24 hours, after which the mixture was concentrated to dryness. The residue was triturated from EtOAc to afford Example 17, 3-hydroxy-4-((6-methoxy-4-(1H-tetrazol-5-yl)pyridin-2-yl)amino)cyclobut-3-ene-1,2-dione (170 mg, 0.59 mmol, 53% yield) as a yellow solid. Data available in Table 3.

### Route F

### Procedure for the Preparation of Example 30, 3′-((2-hydroxy-3,4-dioxocyclobut-1-en-1-yl)amino)-N,N-dimethyl-5′-(1H-tetrazol-5-yl)-[1,1′-biphenyl]-4-carboxamide

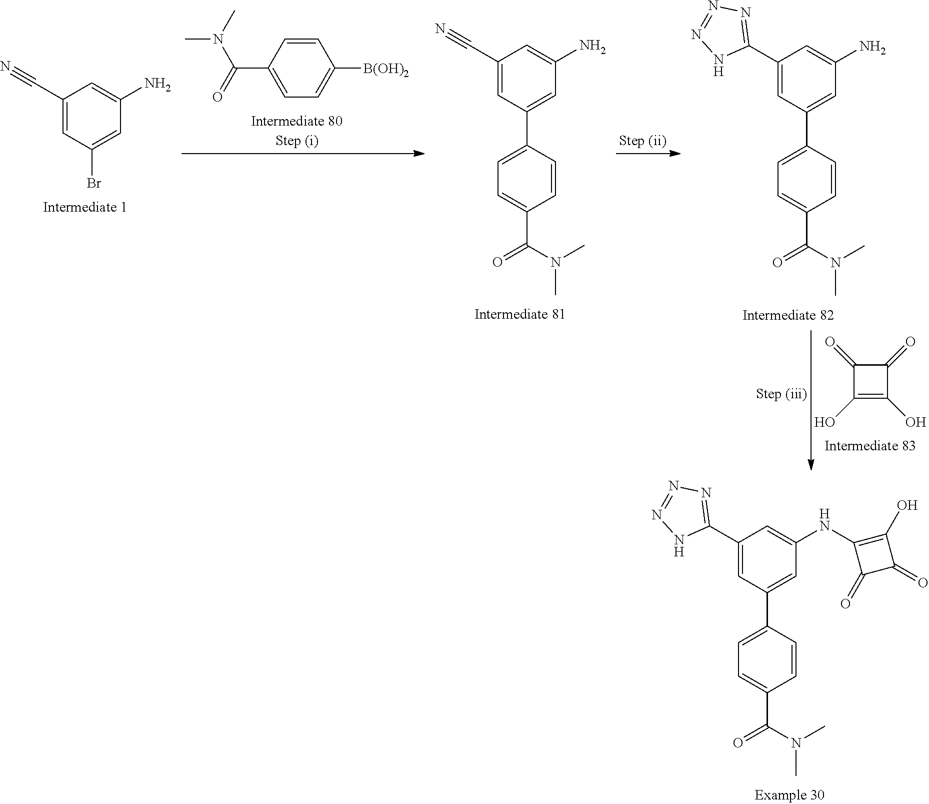 [0282] Step (i): A solution of Intermediate 1, 3-amino-5-bromobenzonitrile (500 mg, 2.53 mmol), Intermediate 80, (4-(dimethylcarbamoyl)phenyl) boronic acid (587 mg, 3.04 mmol), K₂CO₃ (700 mg, 5.07 mmol) and Pd(Ph₃P)₄ (59 mg, 0.050 mmol) in 1,4-dioxane (8 mL) and water (2 mL) was heated to 90° C. for 16 hours. The mixture was cooled and partitioned between EtOAc and water. The organics were separated, and the aqueous layer was washed with EtOAc. The combined organics were dried over Na₂SO₄ and concentrated. The crude material was purified by flash column chromatography (normal phase, silica) under a gradient of EtOAc (0% to 16%) in hexane to afford Intermediate 81, 3′-amino-5′-cyano-N,N-dimethyl-[1,1′-biphenyl]-4-carboxamide (210 mg, 0.79 mmol, 31% yield) as brown gum. Data available in Table 2.
Step (ii): A solution of Intermediate 81, 3′-amino-5′-cyano-N,N-dimethyl-[1,1′-biphenyl]-4-carboxamide (200 mg, 0.754 mmol), NaN₃ (79.9 mg, 1.50 mmol) and NH₄Cl (98 mg, 1.50 mmol) in DMF (2 mL) was heated to 130° C. for 16 hours. The mixture was cooled and acidified to pH 1 with 1 M HCl (aq). The resulting precipitate was collected by filtration and purified by prep HPLC [reversed phase (Sunfire C18, 250×19 mm, 5 μm, 15 mL per min, gradient of Solvent B in Solvent A: 8% to 25% Solvent B (over 22 min), 22 to 25% Solvent B (over 5 min), 100% Solvent B (for 2 min), 100 to 8% Solvent B (over 3 min); Solvent A: 0.1% HCOOH in water. Solvent B: MeCN] to afford Intermediate 82, 3′-amino-N,N-dimethyl-5′-(1H-tetrazol-5-yl)-[1,1′-biphenyl]-4-carboxamide (22 mg, 0.071 mmol, 9% yield) as white solid. Data available in Table 2.
Step (iii): A solution of Intermediate 82, 3′-amino-N,N-dimethyl-5′-(1H-tetrazol-5-yl)-[1,1′-biphenyl]-4-carboxamide (22 mg, 0.071 mmol) and Intermediate 83, squaric acid (8.1 mg, 0.071 mmol) in water (1 mL) was heated to 125° C. in a sealed tube for 6 hours. The mixture was cooled and the solid was collected by filtration to afford Example 30, 3′-((2-hydroxy-3,4-dioxocyclobut-1-en-1-yl)amino)-N,N-dimethyl-5′-(1H-tetrazol-5-yl)-[1,1′-biphenyl]-4-carboxamide (10 mg, 0.025 mmol, 35% yield) as yellow solid. Data available in Table 3.

### EXAMPLES

The invention will now be illustrated, but not limited, by reference to the following examples.

### Examples 1 to 31

The compounds of Examples 1 to 31 shown in Table 1 below have been prepared. Their NMR and LCMS properties and the methods used to prepare them are set out in Table 3. The starting materials for each of the Examples are listed in Table 2.

TABLE 1
Example compounds

| | |
|---|---|
| 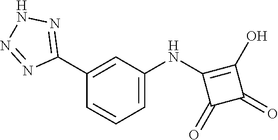 [SMILES: O=c1c(O)c(Nc2cccc(-c3nn[nH]n3)c2)c1=O] | Example 1 |
| 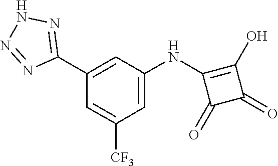 [SMILES: O=c1c(O)c(Nc2cc(-c3nn[nH]n3)cc(C(F)(F)F)c2)c1=O] | Example 2 |
| 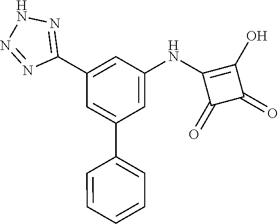 [SMILES: O=c1c(O)c(Nc2cc(-c3ccccc3)cc(-c3nn[nH]n3)c2)c1=O] | Example 3 |
| 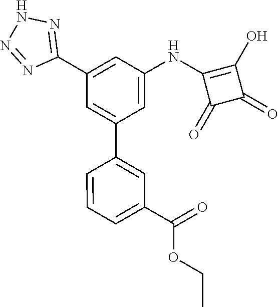 [SMILES: CCOC(=O)c1cccc(-c2cc(Nc3c(O)c(=O)c3=O)cc(-c3nn[nH]n3)c2)c1] | Example 4 |
| 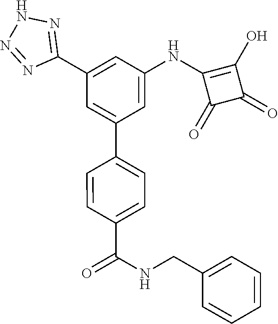 [SMILES: O=C(NCc1ccccc1)c1ccc(-c2cc(Nc3c(O)c(=O)c3=O)cc(-c3nn[nH]n3)c2)cc1] | Example 5 |
| 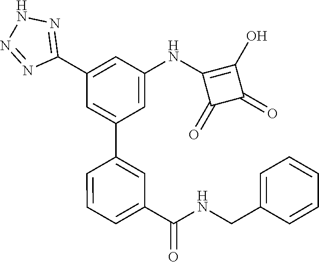 [SMILES: O=C(NCc1ccccc1)c1cccc(-c2cc(Nc3c(O)c(=O)c3=O)cc(-c3nn[nH]n3)c2)c1] | Example 6 |
| 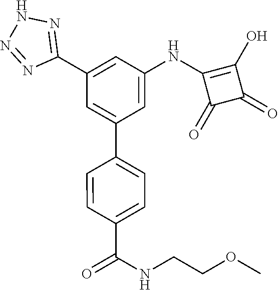 [SMILES: COCCNC(=O)c1ccc(-c2cc(Nc3c(O)c(=O)c3=O)cc(-c3nn[nH]n3)c2)cc1] | Example 7 |
| 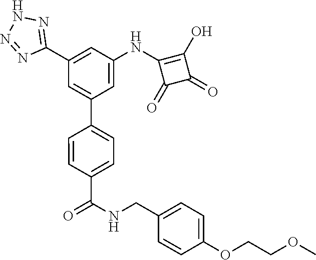 [SMILES: COCCOc1ccc(CNC(=O)c2ccc(-c3cc(Nc4c(O)c(=O)c4=O)cc(-c4nn[nH]n4)c3)cc2)cc1] | Example 8 |
| 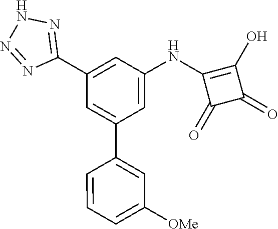 [SMILES: COc1cccc(-c2cc(Nc3c(O)c(=O)c3=O)cc(-c3nn[nH]n3)c2)c1] | Example 9 |
| 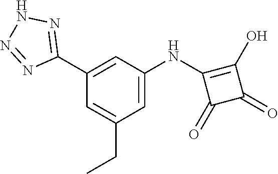 [SMILES: CCc1cc(Nc2c(O)c(=O)c2=O)cc(-c2nn[nH]n2)c1] | Example 10 |
| 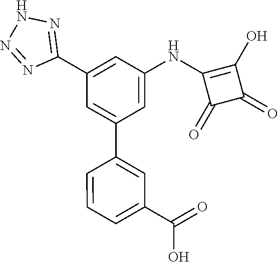 [SMILES: O=C(O)c1cccc(-c2cc(Nc3c(O)c(=O)c3=O)cc(-c3nn[nH]n3)c2)c1] | Example 11 |
| 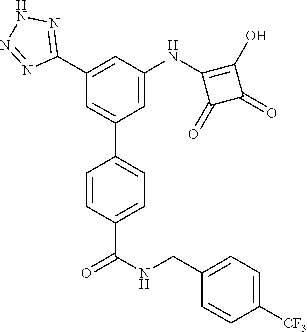 [SMILES: O=C(NCc1ccc(C(F)(F)F)cc1)c1ccc(-c2cc(Nc3c(O)c(=O)c3=O)cc(-c3nn[nH]n3)c2)cc1] | Example 12 |
| 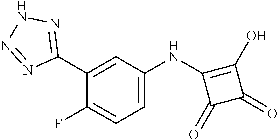 [SMILES: O=c1c(O)c(Nc2ccc(F)c(-c3nn[nH]n3)c2)c1=O] | Example 13 |
| 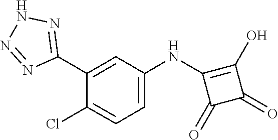 [SMILES: O=c1c(O)c(Nc2ccc(Cl)c(-c3nn[nH]n3)c2)c1=O] | Example 14 |
| 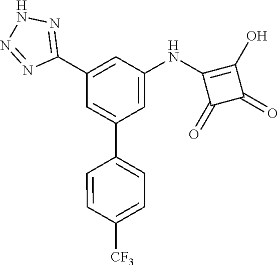 [SMILES: O=c1c(O)c(Nc2cc(-c3ccc(C(F)(F)F)cc3)cc(-c3nn[nH]n3)c2)c1=O] | Example 15 |
| 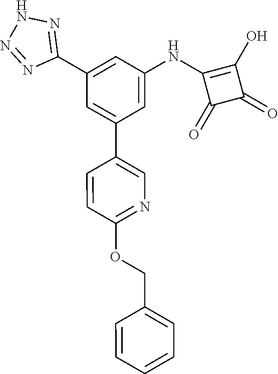 [SMILES: O=c1c(O)c(Nc2cc(-c3ccc(OCc4ccccc4)nc3)cc(-c3nn[nH]n3)c2)c1=O] | Example 16 |
| 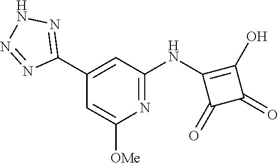 [SMILES: COc1cc(-c2nn[nH]n2)cc(Nc2c(O)c(=O)c2=O)n1] | Example 17 |
| 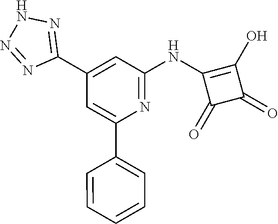 [SMILES: O=c1c(O)c(Nc2cc(-c3nn[nH]n3)cc(-c3ccccc3)n2)c1=O] | Example 18 |
| 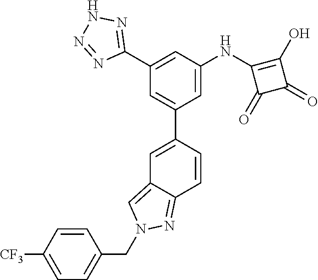 [SMILES: O=c1c(O)c(Nc2cc(-c3ccc4nn(Cc5ccc(C(F)(F)F)cc5)cc4c3)cc(-c3nn[nH]n3)c2)c1=O] | Example 19 |
| 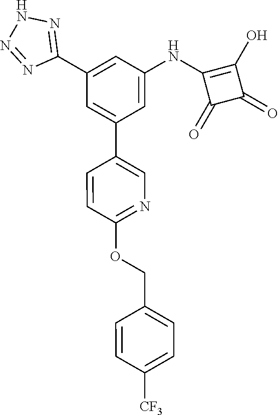 [SMILES: O=c1c(O)c(Nc2cc(-c3ccc(OCc4ccc(C(F)(F)F)cc4)nc3)cc(-c3nn[nH]n3)c2)c1=O] | Example 20 |
| 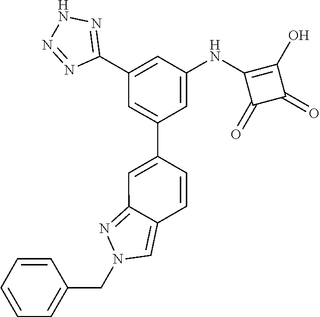 [SMILES: O=c1c(O)c(Nc2cc(-c3ccc4cn(Cc5ccccc5)nc4c3)cc(-c3nn[nH]n3)c2)c1=O] | Example 21 |
| 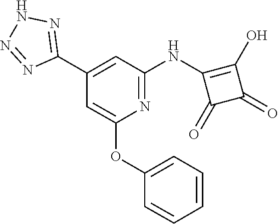 [SMILES: O=c1c(O)c(Nc2cc(-c3nn[nH]n3)cc(Oc3ccccc3)n2)c1=O] | Example 22 |
| 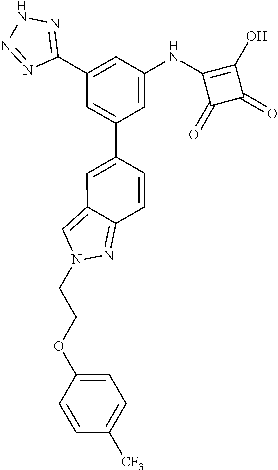 [SMILES: O=c1c(O)c(Nc2cc(-c3ccc4nn(CCOc5ccc(C(F)(F)F)cc5)cc4c3)cc(-c3nn[nH]n3)c2)c1=O] | Example 23 |
| 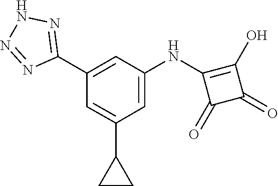 [SMILES: O=c1c(O)c(Nc2cc(-c3nn[nH]n3)cc(C3CC3)c2)c1=O] | Example 24 |
| 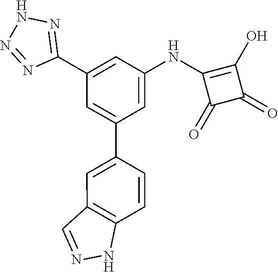 [SMILES: O=c1c(O)c(Nc2cc(-c3ccc4[nH]ncc4c3)cc(-c3nn[nH]n3)c2)c1=O] | Example 25 |
| 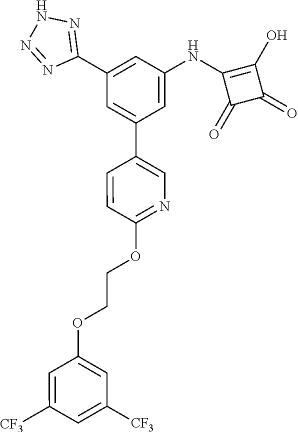 [SMILES: O=c1c(O)c(Nc2cc(-c3ccc(OCCOc4cc(C(F)(F)F)cc(C(F)(F)F)c4)nc3)cc(-c3nn[nH]n3)c2)c1=O] | Example 26 |
| 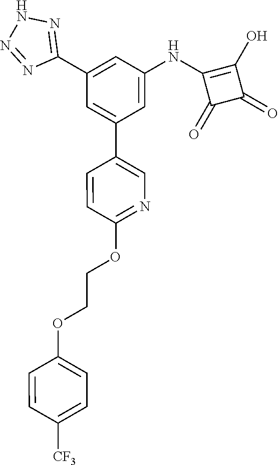 [SMILES: O=c1c(O)c(Nc2cc(-c3ccc(OCCOc4ccc(C(F)(F)F)cc4)nc3)cc(-c3nn[nH]n3)c2)c1=O] | Example 27 |
| 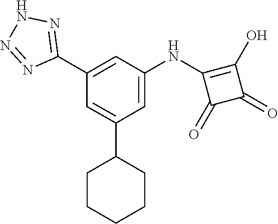 [SMILES: O=c1c(O)c(Nc2cc(-c3nn[nH]n3)cc(C3CCCCC3)c2)c1=O] | Example 28 |
| 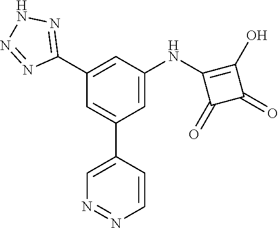 [SMILES: O=c1c(O)c(Nc2cc(-c3ccnnc3)cc(-c3nn[nH]n3)c2)c1=O] | Example 29 |
| 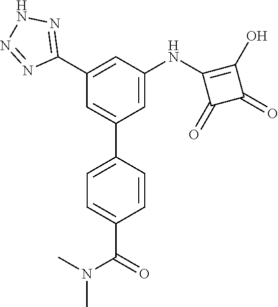 [SMILES: CN(C)C(=O)c1ccc(-c2cc(Nc3c(O)c(=O)c3=O)cc(-c3nn[nH]n3)c2)cc1] | Example 30 |
| 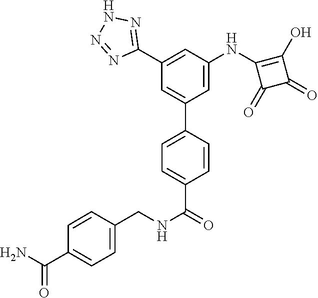 [SMILES: NC(=O)c1ccc(CNC(=O)c2ccc(-c3cc(Nc4c(O)c(=O)c4=O)cc(-c4nn[nH]n4)c3)cc2)cc1] | Example 31 |

TABLE 2
Table 2 -Intermediates

| Intermediate | Route | Name | Data |
|---|---|---|---|
| 1 | | 3-amino-5-bromobenzonitrile | Commercially available, |
| | | | CAS: 49674-16-0 |
| 2 | | phenylboronic acid | Commercially available, |
| | | | CAS: 98-80-6 |
| 3 | Route 1 | 5-amino-[1,1′-biphenyl]-3- | (LC/MS Method A): m/z 193 [M − H]⁻ |
| | | carbonitrile | (ES⁻), at 2.01 min, UV active. |
| 4 | Route 9, | ethyl 3′-amino-5′-cyano-[1,1′- | (LC/MS Method B): m/z 267 [M + H]⁺ |
| | step (i), | biphenyl]-3-carboxylate | (ES⁺), at 1.36 min, UV active. |
| | Intermediate | | |
| | 1 and 84 | | |
| 5 | Route 2 | ethyl 3′-amino-5′-(1H-tetrazol- | (LC/MS Method C): m/z 308 [M − H]⁻ |
| | | 5-yl)-[1,1′-biphenyl]-3- | (ES⁻), at 1.17 min, UV active. |
| | | carboxylate | |
| 6 | | 4-(N- | Commercially available, |
| | | Benzylaminocarbonyl)phenyl- | CAS: 252663-47-1 |
| | | boronic acid | |
| 7 | | 3-Bromo-5-nitrobenzonitrile | Commercially available, |
| | | | CAS: 17601-94-4 |
| 8 | Route 3 | N-benzyl-3′-cyano-5′-nitro- | (LC/MS Method A): m/z 356 [M − H]⁻ |
| | | [1,1′-biphenyl]-4-carboxamide | (ES⁻), at 2.26 min, UV active. |
| 9 | Route 3 | N-benzyl-4-[3-nitro-5-(1H- | (LC/MS Method A): m/z 399 [M − H]⁻ |
| | | tetrazol-5-yl)phenyl]benzamide | (ES⁻), at 1.50 min, UV active. |
| 10 | Route 3 | 4-[3-amino-5-(1H-tetrazol-5- | (LC/MS Method A): m/z 369 [M − H]⁻ |
| | | yl)phenyl]-N-benzyl-benzamide | (ES⁻), at 1.26 min, UV active. |
| 11 | | Diethyl squarate | Commercially available, |
| | | | CAS: 5231-87-8 |
| 12 | Route 3 | N-benzyl-3′-((2-ethoxy-3,4- | (LC/MS Method A): m/z 493 [M − H]⁻ |
| | | dioxocyclobut-1-en-1- | (ES⁻), at 1.43 min, UV active. |
| | | yl)amino)-5′-(1H-tetrazol-5-yl)- | |
| | | [1,1′-biphenyl]-4-carboxamide | |
| 13 | Route 9 | 3′-amino-5′-cyano-[1,1′- | (LC/MS Method H): m/z 239 [M + H]⁺ |
| | | biphenyl]-4-carboxylic acid | (ES⁺), at 1.60 min, UV active. |
| 14 | | 2-methoxyethan-1-amine | Commercially available, |
| | | | CAS: 109-85-3 |
| 15 | Route 4 | 3′-amino-5′-cyano-N-(2- | (LC/MS Method A): m/z 294 [M − H]⁻ |
| | | methoxyethyl)-[1,1′-biphenyl]- | (ES⁻), at 1.74 min, UV active. |
| | | 4-carboxamide | |
| 16 | | 5-amino-2-chlorobenzonitrile | Commercially available, |
| | | | CAS: 5922-60-1 |
| 17 | Route 5 | 4-chloro-3-(1H-tetrazol-5- | (LC/MS Method F): m/z 213 |
| | | yl)aniline | [M + NH₄]⁺ (ES⁺), at 0.42 min, UV |
| | | | active. |
| 18 | | (2-cyclopropyl-4,4,5,5- | Commercially available, |
| | | tetramethyl-1,3,2- | CAS: 126689-01-8 |
| | | dioxaborolane) | |
| 19 | Route 6 | 3-cyclopropyl-5- | (400 MHz, DMSO-d₆) δ: 0.88-0.95 |
| | | nitrobenzonitrile | (m, 2H), 1.07-1.16 (m, 2H), 2.16- |
| | | | 2.26 (m, 1H), 7.99-8.02 (m, 1H), |
| | | | 8.24-8.26 (m, 1H), 8.46-8.48 (m, |
| | | | 1H). |
| 20 | Route 6 | 3-amino-5- | (LC/MS Method G): m/z 159 [M + H]⁺ |
| | | cyclopropylbenzonitrile | (ES⁺), at 2.04 min, UV active. |
| 21 | Route 6 | 3-cyclopropyl-5-(1H-tetrazol-5- | (LC/MS Method G): m/z 202 [M + H]⁺ |
| | | yl)aniline | (ES⁺), at 1.47 min, UV active. |
| 22 | Route 6, | 5-nitro-2′,3′,4′,5′-tetrahydro- | (400 MHz, DMSO-d₆) δ: 1.56-1.65 |
| | step (i), | [1,1′-biphenyl]-3-carbonitrile | (m, 2H), 1.69-1.78 (m, 2H), 2.19- |
| | intermediates | | 2.28 (m, 2H), 2.37-2.46 (m, 2H), |
| | 7 and 85 | | 6.51-6.58 (m, 1H), 8.33-8.36 (m, |
| | | | 1H), 8.39-8.42 (m, 1H), 8.56-8.59 |
| | | | (m, 1H) |
| 23 | Route 7 | 3-amino-5- | (LC/MS Method G): m/z 201 [M + H]⁺ |
| | | cyclohexylbenzonitrile | (ES⁺), at 2.59 min, UV active. |
| 24 | | 4-(tributylstannyl)pyridazine | Commercially available, |
| | | | CAS: 194865-89-9 |
| 25 | Route 8 | 3-nitro-5-(pyridazin-4- | (LC/MS Method G): m/z 227 [M + H]⁺ |
| | | yl)benzonitrile | (ES⁺), at 1.66 min, UV active. |
| 26 | | (4-(ethoxycarbonyl) phenyl) | Commercially available, |
| | | boronic acid | CAS: 4334-88-7 |
| 27 | Route 9 | ethyl 3′-amino-5′-cyano-[1,1′- | (LC/MS Method H): m/z 267 [M + H]⁺ |
| | | biphenyl]-4-carboxylate | (ES⁺), at 2.07 min, UV active. |
| 28 | Route 9 | 3′-amino-5′-(1H-tetrazol-5-yl)- | (LC/MS Method H): m/z 282 [M + H]⁺ |
| | | [1,1′-biphenyl]-4-carboxylic | (ES⁺), at 1.42 min, UV active. |
| | | acid | |
| 29 | Route 9 | 3′-((2-ethoxy-3,4- | (LC/MS Method I): m/z 406 [M + H]⁺ |
| | | dioxocyclobut-1-en-1- | (ES⁺), at 1.61 min, UV active. |
| | | yl)amino)-5′-(1H-tetrazol-5-yl)- | |
| | | [1,1′-biphenyl]-4-carboxylic | |
| | | acid | |
| 30 | Route 10 | 3-bromo-5-(1H-tetrazol-5- | (LC/MS Method K): m/z 242 [M + H]⁺ |
| | | yl)aniline | (ES⁺), at 2.94 min, UV active. |
| 31 | Route 10 | 3-((3-bromo-5-(1H-tetrazol-5- | (LC/MS Method K): m/z 381 |
| | | yl)phenyl)amino)-4- | [M + NH₄]⁺ (ES⁺), at 3.89 min, UV |
| | | ethoxycyclobut-3-ene-1,2- | active. |
| | | dione | |
| 32 | | 5-bromo-2-fluoropyridine | Commercially available, |
| | | | CAS: 766-11-0 |
| 33 | | [4-(trifluoromethyl)phenyl] | Commercially available, |
| | | methanol | CAS: 349-95-1 |
| 34 | Route 11 | (5-bromo-2-((4- | (LC/MS Method G): m/z 331 [M + H]⁺ |
| | | (trifluoromethyl)benzyl)oxy)pyri- | (ES⁺), at 2.98 min, UV active. |
| | | dine) | |
| 35 | Route 11 | (5-(4,4,5,5-tetramethyl-1,3,2- | (LC/MS Method G): m/z 381 [M + H]⁺ |
| | | dioxaborolan-2-yl)-2-((4- | (ES⁺), at 3.15 min, UV active. |
| | | (trifluoromethyl)benzyl)oxy)pyri- | |
| | | dine) | |
| 36 | | 5-bromo-2-nitrobenzaldehyde | Commercially available, |
| | | | CAS: 20357-20-4 |
| 37 | | benzylamine | Commercially available, |
| | | | CAS: 100-46-9 |
| 38 | Route 12 | N-benzyl-1-(5-bromo-2- | (LC/MS Method K): m/z 320 [M + H]⁺ |
| | | nitrophenyl)methanimine | (ES⁺), at 4.87 min, UV active. |
| 39 | Route 12 | 2-benzyl-5-bromo-2H-indazole | (LC/MS Method G): m/z 288 [M + H]⁺ |
| | | | (ES⁺), at 2.45 min, UV active. |
| 40 | Route 12 | 2-benzyl-5-(4,4,5,5- | (LC/MS Method G): m/z 335 [M + H]⁺ |
| | | tetramethyl-1,3,2- | (ES⁺), at 2.75 min, UV active. |
| | | dioxaborolan-2-yl)-2H-indazole | |
| 41 | | 4-(trifluoromethyl)phenol | Commercially available, |
| | | | CAS: 402-45-9 |
| 42 | | tert-butyl (2- | Commercially available, |
| | | bromoethyl)carbamate | CAS: 39684-80-5 |
| 43 | Route 13 | tert-butyl (2-(4- | (LC/MS Method A): m/z 206 [M + H − |
| | | (trifluoromethyl)phenoxy)ethyl) | Boc]⁺ (ES⁺), at 2.42 min, UV active |
| | | carbamate | |
| 44 | Route 13 | 2-(4- | (LC/MS Method A): m/z 206 [M + H − |
| | | (trifluoromethyl)phenoxy)ethan- | Boc]⁺ (ES⁺), at 1.92 min, UV active |
| | | 1-amine, hydrochloride salt | |
| 45 | | 3,5-bis(trifluoromethyl)phenol | Commercially available, |
| | | | CAS: 349-58-6 |
| 46 | | 2-bromoethan-1-ol | Commercially available, |
| | | | CAS: 540-51-2 |
| 47 | Route 14 | 2-(3,5-bis(trifluoromethyl)phe- | (LC/MS Method G): m/z 275 [M + H]⁺ |
| | | noxy)ethan-1-ol | (ES⁺), at 2.31 min, UV active. |
| 48 | Route 14 | 2-(2-(3,5- | (LC/MS Method G): m/z 431 [M + H]⁺ |
| | | bis(trifluoromethyl)phe- | (ES⁺), at 3.20 min, UV active. |
| | | noxy)ethoxy)-5-bromopyridine | |
| 49 | | 2-((tert- | Commercially available, |
| | | butyldimethylsilyl)oxy)ethan-1-ol | CAS: 102229-10-7 |
| 50 | Route 15 | tert-butyldimethyl(2-(4- | ¹H NMR(CDCl₃, 400 MHz): δ (ppm) |
| | | (trifluoromethyl)phenoxy)eth- | 0.12 (s, 6H), 0.93 (s, 9H), 3.98-4.03 |
| | | oxy)silane | (m, 2H), 4.08-4.12 (m, 2H), 6.99 (d, |
| | | | J = 8.4 Hz, 2H), 7.56 (d, J = 8.2 Hz, |
| | | | 1H). |
| 51 | Route 15 | 2-(4- | (LC/MS Method A): m/z 207 [M + H − |
| | | (trifluoromethyl)phenoxy)ethan- | Boc]⁺ (ES⁺), at 2.09 min, UV active |
| | | 1-ol | |
| 52 | | 4-boronobenzoic acid | Commercially available, |
| | | | CAS: 14047-29-1 |
| 53 | | 4-(aminomethyl) benzamide | Commercially available, |
| | | | CAS: 369-53-9 |
| 54 | Route 16 | (4-((4- | (LC/MS Method H): m/z 299 [M + H]⁺ |
| | | carbamoylbenzyl)carbamoyl)phe- | (ES⁺), at 1.24 min, UV active. |
| | | nyl)boronic acid | |
| 55 | | 2,6-dichloroisonicotinonitrile | Commercially available, |
| | | | CAS: 32710-65-9 |
| 56 | | Phenol | Commercially available, |
| | | | CAS: 108-95-2 |
| 57 | Route 17 | 2-chloro-6- | (LC/MS Method L): m/z 231 [M + H]⁺ |
| | | phenoxyisonicotinonitrile | (ES⁺), at 3.61 min, UV active. |
| 58 | | 4-methoxybenzylamine | Commercially available, |
| | | | CAS: 2393-23-9 |
| 59 | Route 17 | 2-((4-methoxybenzyl)amino)-6- | (LC/MS Method L): m/z 332 [M + H]⁺ |
| | | phenoxyisonicotinonitrile | (ES⁺), at 3.95 min, UV active. |
| 60 | Route 17 | N-(4-methoxybenzyl)-6- | (LC/MS Method L): m/z 375 [M + H]⁺ |
| | | phenoxy-4-(1H-tetrazol-5- | (ES⁺), at 3.35 min, UV active. |
| | | yl)pyridin-2-amine | |
| 61 | Route 18 | 6-phenoxy-4-(1H-tetrazol-5- | (LC/MS Method L): m/z 255 [M + H]⁺ |
| | | yl)pyridin-2-amine | (ES⁺), at 2.08 min, UV active. |
| 62 | Route 19 | 2-chloro-6-((4-methoxy- | (LC/MS Method L): m/z 274 [M + H]⁺ |
| | | benzyl)amino)isonicotinonitrile | (ES⁺), at 3.65 min, UV active. |
| 63 | Route 19 | 6-chloro-N-(4-methoxybenzyl)- | (LC/MS Method L): m/z 317 [M + H]⁺ |
| | | 4-(1H-tetrazol-5-yl)pyridin-2- | (ES⁺), at 3.05 min, UV active. |
| | | amin | |
| 64 | Route 20 | 6-methoxy-N-(4- | (LC/MS Method L): m/z 313 [M + H]⁺ |
| | | methoxybenzyl)-4-(1H-tetrazol- | (ES⁺), at 2.80 min, UV active. |
| | | 5-yl)pyridin-2-amine | |
| 65 | Route 21 | N-(4-methoxybenzyl)-6-phenyl- | (LC/MS Method L): m/z 359 [M + H]⁺ |
| | | 4-(1H-tetrazol-5-yl)pyridin-2- | (ES⁺), at 2.77 min, UV active. |
| | | amine | |
| 66 | | 4,4,5,5-tetramethyl-2-vinyl- | Commercially available, |
| | | 1,3,2-dioxaborolane | CAS: 75927-49-0 |
| 67 | Route 22 | 3-amino-5-vinylbenzonitrile | (LC/MS Method A): m/z 143 [M − H]⁻ |
| | | | (ES⁺), at 1.68 min, UV active. |
| 68 | | benzyl chloroformate | Commercially available, |
| | | | CAS: 501-53-1 |
| 69 | Route 22 | benzyl (3-cyano-5- | (LC/MS Method B): m/z 279 [M − H]⁻ |
| | | vinylphenyl) carbamate | (ES⁺), at 1.50 min, UV active |
| 70 | Route 22 | benzyl (3-(1H-tetrazol-5-yl)-5- | (LC/MS Method C): m/z 320 [M − H]⁻ |
| | | vinylphenyl)carbamate | (ES⁺), at 1.29 min, UV active. |
| 71 | Route 22 | 3-ethoxy-4-((3-ethyl-5-(1H- | (LC/MS Method C): m/z 312 [M − H]⁻ |
| | | tetrazol-5- | (ES⁺), at 1.14 min, UV active. |
| | | yl)phenyl)amino)cyclobut-3- | |
| | | ene-1,2-dione | |
| 72 | | 3-amino-5- | Commercially available, |
| | | (trifluoromethyl)benzonitrile | CAS: 49674-28-4 |
| 73 | Route A | 3-(1H-tetrazol-5-yl)-5- | (LC/MS Method C): m/z 227 [M − H]⁻ |
| | | (trifluoromethyl)aniline | (ES⁻), at 1.01 min, UV active. |
| 74 | Route A | 3-((3-(1H-tetrazol-5-yl)-5- | (LC/MS Method C): m/z 351 [M − H]⁻ |
| | | (trifluoromethyl)phenyl)amino)- | (ES⁻), at 1.28 min, UV active. |
| | | 4-ethoxycyclobut-3-ene-1,2- | |
| | | dione | |
| 75 | | (4-(2- | Commercially available, |
| | | methoxyethoxy)phenyl)methan | CAS: 102196-20-3 |
| | | amine | |
| 76 | Route C | 3′-((2-ethoxy-3,4- | (LC/MS Method C): m/z 567 [M − H]⁻ |
| | | dioxocyclobut-1-en-1- | (ES⁻), at 1.25 min, UV active. |
| | | yl)amino)-N-(4-(2- | |
| | | methoxyethoxy)benzyl)-5′-(1H- | |
| | | tetrazol-5-yl)-[1,1′-biphenyl]-4- | |
| | | carboxamide | |
| 77 | | 4,4,5,5-tetramethyl-2-(4- | Commercially available, |
| | | (trifluoromethyl)phenyl)-1,3,2- | CAS: 214360-65-3 |
| | | dioxaborolane | |
| 78 | Route 18, | 6-methoxy-4-(1H-tetrazol-5- | (LC/MS Method L): m/z 193 [M + H]⁺ |
| | Intermediate | yl)pyridin-2-amine | (ES⁺), at 1.01 min, UV active. |
| | 64 | | |
| 79 | Route E | 3-ethoxy-4-((6-methoxy-4-(1H- | (LC/MS Method L): m/z 317 [M + H]⁺ |
| | | tetrazol-5-yl)pyridin-2- | (ES⁺), at 2.19 min, UV active. |
| | | yl)amino)cyclobut-3-ene-1,2- | |
| | | dione | |
| 80 | | (4-(dimethylcarbamoyl)phe- | Commercially available, |
| | | nyl)boronic acid | CAS: 405520-68-5 |
| 81 | Route F | 3′-amino-5′-cyano-N,N- | (LC/MS Method H): m/z 266 [M + H]⁺ |
| | | dimethyl-[1,1′-biphenyl]-4- | (ES⁺), at 1.43 min, UV active. |
| | | carboxamide | |
| 82 | Route F | 3′-amino-N,N-dimethyl-5′-(1H- | (LC/MS Method H): m/z 309 [M + H]⁺ |
| | | tetrazol-5-yl)-[1,1′-biphenyl]-4- | (ES⁺), at 1.40 min, UV active. |
| | | carboxamide | |
| 83 | | Squaric acid | Commercially available, |
| | | | CAS: 2892-51-5 |
| 84 | | 3- | Commercially available, |
| | | Ethoxycarbonylphenylboronic | CAS: 4334-87-6 |
| | | acid | |
| 85 | | 2-(1-Cyclohexenyl)-4,4,5,5- | Commercially available, |
| | | tetramethyl-1,3,2- | CAS: 141091-37-4 |
| | | dioxaborolane | |
| 86 | | 5-(3-Aminophenyl)tetrazole | Commercially available, |
| | | | CAS: 73732-51-1 |
| 87 | Route 9, | 3′-((2-ethoxy-3,4- | (LC/MS Method C): m/z 406 [M − H]⁻ |
| | Intermediates | dioxocyclobut-1-en-1- | (ES⁻), at 1.08 min, UV active. |
| | 1 and 84 | yl)amino)-5′-(1H-tetrazol-5-yl)- | |
| | | [1,1′-biphenyl]-3-carboxylic | |
| | | acid | |
| 88 | Route 2, | 3′-amino-N-(2-methoxyethyl)- | (LC/MS Method A): m/z 337 [M − H]⁻ |
| | intermediate | 5′-(1H-tetrazol-5-yl)-[1,1′- | (ES⁻), at 1.54 min, UV active. |
| | 15 | biphenyl]-4-carboxamide | |
| 89 | | 3-Methoxyphenylboronic | Commercially available, |
| | | | CAS: 10365-98-7 |
| 90 | | 4-(trifluoromethyl)benzylamine | Commercially available, |
| | | | CAS: 3300-51-4 |
| 91 | Route 5, | 4-fluoro-3-(1H-tetrazol-5- | (LC/MS Method F): m/z does not |
| | intermediate | yl)aniline | align with structure, at 0.69 min, UV |
| | 92 | | active. |
| 92 | | 5-amino-2-fluorobenzonitrile | Commercially available, |
| | | | CAS: 53312-81-5 |
| 93 | | 2-(Benzyloxy)-5-(4,4,5,5- | Commercially available, |
| | | tetramethyl-1,3,2- | CAS: 832735-54-3 |
| | | dioxaborolan-2-yl)pyridine | |
| 94 | Route 18, | 6-phenyl-4-(1H-tetrazol-5-yl) | (LC/MS Method L): m/z 239 [M + H]⁺ |
| | Intermediate | pyridin-2-amine | (ES⁺), at 1.32 min, UV active. |
| | 65 | | |
| 95 | Route 12, | 5-(4,4,5,5-tetramethyl-1,3,2- | (LC/MS Method G): m/z 403 [M + H]⁺ |
| | Intermediates | dioxaborolan-2-yl)-2-(4- | (ES⁺), at 2.83 min, UV active. |
| | 36 and 90 | (trifluoromethyl)benzyl)-2H- | |
| | | indazole | |
| 96 | Route 12, | 5-(4,4,5,5-tetramethyl-1,3,2- | (LC/MS Method G): m/z 433 [M + H]⁺ |
| | Intermediates | dioxaborolan-2-yl)-2-(2-(4- | (ES⁺), at 2.94 min, UV active. |
| | 36 and 44 | (trifluoromethyl)phenoxy)ethyl)- | |
| | | 2H-indazole | |
| 97 | | 5-(4,4,5,5-Tetramethyl-1,3,2- | Commercially available, |
| | | dioxaborolan-2-yl)-1H-indazole | CAS: 862723-42-0 |
| 98 | Route 11, | 2-(2-(3,5- | (LC/MS Method G): m/z 478 [M + H]⁺ |
| | step (ii), | bis(trifluoromethyl)phenoxy)eth- | (ES⁺), at 3.42 min, UV active (boronic |
| | Intermediate | oxy)-5-(4,4,5,5-tetramethyl- | ester); m/z 396 [M + H]⁺ (ES⁺), at 2.54 |
| | 48 | 1,3,2-dioxaborolan-2-yl) | min, UV active (boronic acid) |
| | | pyridine | |
| 99 | Route 11, | (6-(2-(4- | (LC/MS Method G): m/z 328 [M + H]⁺ |
| | step (ii), | (trifluoromethyl)phenoxy)eth- | (ES⁺), at 2.32 min, UV active. |
| | Intermediate | oxy)pyridin-3-yl)boronic acid | |
| | 100 | | |
| 100 | Route 14, | 5-bromo-2-(2-(4- | (LC/MS Method G): m/z 363 [M + H]⁺ |
| | step (ii) | (trifluoromethyl)phenoxy)eth- | (ES⁺), at 3.01 min, UV active. |
| | Intermediates | oxy)pyridine | |
| | 32 and 51 | | |
| 101 | Route 6, | 3-cyclohexyl-5-(1H-tetrazol-5- | (LC/MS Method G): m/z 244 [M + H]⁺ |
| | Step (iii), | yl)aniline | (ES⁺), at 2.02 min, UV active. |
| | Intermediate | | |
| | 23 | | |
| 102 | Route 6, steps | 3-(pyridazin-4-yl)-5-(1H- | (LC/MS Method G): m/z 240 [M + H]⁺ |
| | (ii)-(iii), | tetrazol-5-yl)aniline | (ES⁺), at 1.23 min, UV active. |
| | Intermediate | | |
| | 25 | | |

TABLE 3
Table 3 - Example compounds

| Ex. | | | Synthetic | | LCMS | |
|---|---|---|---|---|---|---|
| No. | Name | Intermediate | method | ¹H NMR | Method | LCMS data |
| 1 | 3-((3-(1H-tetrazol-5- | 86 and 11 | Route A, | (400 MHz, DMSO-d₆) | D | m/z 255 (M − |
| | yl)phenyl)amino)-4- | | steps (ii)- | δ: 7.50-7.54 (m, 1H), | | H)⁻ (ES⁻) at |
| | hydroxycyclobut-3- | | (iii) | 7.59-7.61 (m, 1H), | | 1.19 min, |
| | ene-1,2-dione | | | 7.66-7.69 (m, 1H), | | UV active |
| | | | | 8.09-8.10 (m, 1H), | | |
| | | | | 10.49 (s, 1H). Two | | |
| | | | | acidic protons not | | |
| | | | | observed. | | |
| 2 | 3-((3-(1H-tetrazol-5- | 72 | Route A | (400 MHz, DMSO-d₆) | D | m/z 324 (M − |
| | yl)-5-(trifluorometh- | | | δ: 7.85 (s, 1H), 8.20 | | H)⁻ (ES⁻) at |
| | yl)phenyl)amino)- | | | (s, 1H), 8.37 (s, 1H), | | 2.12 min, |
| | 4-hydroxycyclobut-3- | | | 10.54 (s, 1H). Two | | UV active |
| | ene-1,2-dione | | | acidic protons not | | |
| | | | | observed. | | |
| 3 | 3-((5-(1H-tetrazol-5- | 3 | Route A | (400 MHz, DMSO-d₆) | D | m/z 331 (M − |
| | yl)-[1,1′-biphenyl]-3- | | | δ: 7.41-7.45 (m, 1H), | | H)⁻ (ES⁻) at |
| | yl)amino)-4- | | | 7.51-7.55 (m, 2H), | | 2.41 min, |
| | hydroxycyclobut-3- | | | 7.74-7.77 (m, 2H), | | UV active |
| | ene-1,2-dione | | | 9.97-9.98 (m, 1H), | | |
| | | | | 8.02-8.03 (m, 1H), | | |
| | | | | 8.05-8.06 (m, 1H), | | |
| | | | | 10.70 (s, 1H). Two | | |
| | | | | acidic protons not | | |
| | | | | observed. | | |
| 4 | ethyl 3′-((2-hydroxy- | 5 and 11 | Route A, | (400 MHz, DMSO-d₆) | D | m/z 404 (M − |
| | 3,4-dioxocyclobut-1- | | steps (ii)- | δ: 1.35 (t, J = 7.1 Hz, | | H)⁻ (ES⁻) at |
| | en-1-yl)amino)-5′- | | (iii) | 3H), 4.37 (q, J = 7.1 | | 2.74 min, |
| | (1H-tetrazol-5-yl)- | | | Hz, 2H), 7.67-7.71 (m, | | UV active |
| | [1,1′-biphenyl]-3- | | | 1H), 7.95-7.96 (m, | | |
| | carboxylate | | | 1H), 8.00-8.05 (m, | | |
| | | | | 2H), 8.11-8.12 (m, | | |
| | | | | 1H), 8.14-8.15 (m, | | |
| | | | | 1H), 8.32-8.33 (m, | | |
| | | | | 1H), 10.51 (s, 1H). | | |
| | | | | Two acidic protons not | | |
| | | | | observed. | | |
| 5 | N-benzyl-3′-((2- | 12 | Route B | (400 MHz, DMSO-d₆) | D | m/z 465 (M − |
| | hydroxy-3,4- | | | δ: 4.51 (d, J = 6.0 Hz, | | H)⁻ (ES⁻) at |
| | dioxocyclobut-1-en- | | | 2H), 7.20-7.27 (m, | | 2.65 min, |
| | 1-yl)amino)-5′-(1H- | | | 1H), 7.30-7.36 (m, | | UV active |
| | tetrazol-5-yl)-[1,1′- | | | 4H), 7.87 (d, J = 8.4 | | |
| | biphenyl]-4- | | | Hz, 2H), 8.02-8.07 (m, | | |
| | carboxamide | | | 3H), 8.07-8.12 (m, | | |
| | | | | 2H), 9.14 (t, J = 6.0 | | |
| | | | | Hz, 1H), 10.74 (s, 1H). | | |
| | | | | Two acidic protons not | | |
| | | | | observed. | | |
| 6 | N-benzyl-3′-((2- | 87 and 37 | Route C | (400 MHz, DMSO-d₆) | D | m/z 465 (M − |
| | hydroxy-3,4- | | | δ: 4.51 (d, J = 5.9 Hz, | | H)⁻ (ES⁻) at |
| | dioxocyclobut-1-en- | | | 2H), 7.20-7.25 (m, | | 2.70 min, |
| | 1-yl)amino)-5′-(1H- | | | 1H), 7.30-7.35 (m, | | UV active |
| | tetrazol-5-yl)-[1,1′- | | | 4H), 7.60-7.64 (m, | | |
| | biphenyl]-3- | | | 1H), 7.90-7.95 (m, | | |
| | carboxamide | | | 3H), 8.08-8.09 (m, | | |
| | | | | 1H), 8.15-8.16 (m, | | |
| | | | | 1H), 8.27-8.28 (m, | | |
| | | | | 1H), 9.17 (t, J = 5.9 | | |
| | | | | Hz, 1H), 10.38 (s, 1H). | | |
| | | | | Two acidic protons not | | |
| | | | | observed. | | |
| 7 | 3′-((2-hydroxy-3,4- | 88 and 11 | Route A, | (400 MHz, DMSO-d₆) | D | m/z 433 (M − |
| | dioxocyclobut-1-en- | | steps (ii)- | δ: 3.23-3.29 (m, 3H), | | H)⁻ (ES⁻) at |
| | 1-yl)amino)-N-(2- | | (iii) | 3.45 (m, 4H), 7.81- | | 2.08 min, |
| | methoxyethyl)-5′- | | | 7.88 (m, 2H), 7.90 (s, | | UV active |
| | (1H-tetrazol-5-yl)- | | | 1H), 7.96-7.99 (m, | | |
| | [1,1′-biphenyl]-4- | | | 2H), 8.07 (s, 1H), 8.27 | | |
| | carboxamide | | | (s, 1H), 8.56-8.63 (m, | | |
| | | | | 1H), 10.24 (s, 1H). | | |
| | | | | Two acidic protons not | | |
| | | | | observed. | | |
| 8 | 3′-((2-hydroxy-3,4- | 29 and 75 | Route C | (400 MHz, DMSO-d₆) | D | m/z 539 (M − |
| | dioxocyclobut-1-en- | | | δ: 3.27-3.28 (m, 3H), | | H)⁻ (ES⁻) at |
| | 1-yl)amino)-N-(4-(2- | | | 3.60-3.64 (m, 2H), | | 2.72 min, |
| | methoxyethoxy)benzyl)- | | | 4.02-4.06 (m, 2H), | | UV active |
| | 5′-(1H-tetrazol- | | | 4.42 (d, J = 5.9 Hz, | | |
| | 5-yl)-[1,1′-biphenyl]- | | | 2H), 6.89 (d, J = 8.5 | | |
| | 4-carboxamide | | | Hz, 2H), 7.24 (d, J = | | |
| | | | | 8.5 Hz, 2H), 7.85 (d, J = | | |
| | | | | 8.3 Hz, 2H), 7.93 (s, | | |
| | | | | 1H), 8.02 (d, J = 8.3 | | |
| | | | | Hz, 2H), 8.07 (s, 1H), | | |
| | | | | 8.23 (s, 1H), 9.05 (t, J = | | |
| | | | | 5.9 Hz, 1H), 10.35 | | |
| | | | | (s, 1H). Two acidic | | |
| | | | | protons not observed. | | |
| 9 | 3-hydroxy-4-((3′- | 31 and 89 | Route D | (400 MHz, DMSO-d₆) | D | m/z 362 (M − |
| | methoxy-5-(1H- | | | δ: 3.84 (s, 3H), 6.96- | | H)⁻ (ES⁻) at |
| | tetrazol-5-yl)-[1,1′- | | | 7.02 (m, 1H), 7.32- | | 2.51 min, |
| | biphenyl]-3- | | | 7.35 (m, 2H), 7.41- | | UV active |
| | yl)amino)cyclobut-3- | | | 7.45 (m, 1H), 7.91- | | |
| | ene-1,2-dione | | | 7.92 (m, 1H), 8.03- | | |
| | | | | 8.04 (m, 1H), 8.13- | | |
| | | | | 8.14 (m, 1H), 10.49 (s, | | |
| | | | | 1H). Two acidic | | |
| | | | | protons not observed. | | |
| 10 | 3-((3-ethyl-5-(1H- | 71 | Route A, | (400 MHz, DMSO-d₆) | D | m/z 284 (M − |
| | tetrazol-5- | | step (iii) | δ: 1.23 (t, J = 7.3 Hz, | | H)⁻ (ES⁻) at |
| | yl)phenyl)amino)-4- | | | 3H), 2.66 (q, J = 7.3 | | 1.97 min, |
| | hydroxycyclobut-3- | | | Hz, 2H), 7.50 (s, 1H), | | UV active |
| | ene-1,2-dione | | | 7.54-7.55 (m, 1H), | | |
| | | | | 7.88-7.90 (m, 1H), | | |
| | | | | 10.46 (s, 1H). Two | | |
| | | | | acidic protons not | | |
| | | | | observed. | | |
| 11 | 3′-((2-hydroxy-3,4- | 87 | Route A, | (400 MHz, DMSO-d₆) | D | m/z 376 (M − |
| | dioxocyclobut-1-en- | | step (iii) | δ: 7.59-7.71 (m, 1H), | | H)⁻ (ES⁻) at |
| | 1-yl)amino)-5′-(1H- | | | 7.91-7.96 (m, 1H), | | 2.09 min, |
| | tetrazol-5-yl)-[1,1′- | | | 7.97-8.03 (m, 2H), | | UV active |
| | biphenyl]-3- | | | 8.08-8.12 (m, 1H), | | |
| | carboxylic acid | | | 8.14-8.21 (m, 1H), | | |
| | | | | 8.34 (s, 1H), 10.40 (s, | | |
| | | | | 1H). Three acidic | | |
| | | | | protons not observed. | | |
| 12 | 3′-((2-hydroxy-3,4- | 29 and 90 | Route C | (400 MHz, DMSO-d₆) | E | m/z 533 (M − |
| | dioxocyclobut-1-en- | | | δ: 4.61 (d, J = 6.1 Hz, | | H)⁻ (ES⁻) at |
| | 1-yl)amino)-5′-(1H- | | | 2H), 7.58 (d, J = 8.1 | | 1.68 min, |
| | tetrazol-5-yl)-N-(4- | | | Hz, 2H), 7.72 (d, J = | | UV active |
| | (trifluoromethyl)benzyl)- | | | 8.1 Hz, 2H), 7.81 (s, | | |
| | [1,1′-biphenyl]- | | | 1H), 7.90 (d, J = 8.2 | | |
| | 4-carboxamide | | | Hz, 2H), 8.01-8.08 (m, | | |
| | | | | 3H), 8.50 (s, 1H), 9.26 | | |
| | | | | (t, J = 6.1 Hz, 1H), | | |
| | | | | 9.65 (s, 1H). Two | | |
| | | | | acidic protons not | | |
| | | | | observed. | | |
| 13 | 3-((4-fluoro-3-(1H- | 91 and 11 | Route A, | (400 MHz, DMSO-d₆) | F | m/z 276 |
| | tetrazol-5- | | steps (ii)- | δ: 7.24-7.35 (m, 1H), | | (M + H)⁺ |
| | yl)phenyl)amino)-4- | | (iii) | 7.64-7.73 (m, 1H), | | (ES⁺) at |
| | hydroxycyclobut-3- | | | 8.07-8.16 (m, 1H), | | 0.69 min, |
| | ene-1,2-dione | | | 10.62 (s, 1H). Two | | UV active |
| | | | | acidic protons not | | |
| | | | | observed. | | |
| 14 | 3-((4-chloro-3-(1H- | 17 and 11 | Route A, | (400 MHz, DMSO-d₆) | F | m/z 290 (M − |
| | tetrazol-5- | | steps (ii)- | δ: 7.61-7.75 (m, 2H), | | H)⁻ (ES⁻) at |
| | yl)phenyl)amino)-4- | | (iii) | 7.88 (s, 1H), 10.60 (s, | | 0.78 min, |
| | hydroxycyclobut-3- | | | 1H). Two acidic | | UV active |
| | ene-1,2-dione | | | protons not observed. | | |
| 15 | 3-((5-(1H-tetrazol-5- | 31 and 77 | Route D | (400 MHz, DMSO-d₆) | K | m/z 402 |
| | yl)-4′- | | | δ: 7.80-7.83 (m, 1H), | | (M + H)⁺ |
| | (trifluoromethyl)- | | | 7.87 (d, J = 8.2 Hz, | | (ES⁺) at |
| | [1,1′-biphenyl]-3- | | | 2H), 7.99 (d, J = 8.2 | | 3.79 min, |
| | yl)amino)-4- | | | Hz, 2H), 8.03-8.05 (m, | | UV active |
| | hydroxycyclobut-3- | | | 1H), 8.38-8.45 (m, | | |
| | ene-1,2-dione | | | 1H), 9.63 (s, 1H). Two | | |
| | | | | acidic protons not | | |
| | | | | observed. Ammonium | | |
| | | | | signal observed at | | |
| | | | | 6.95-7.20 ppm. | | |
| 16 | 3-((3-(6- | 31 and 93 | Route D | (400 MHz, DMSO-d₆) | K | m/z 441 |
| | (benzyloxy)pyridin- | | | δ: 5.43 (s, 2H), 7.04 | | (M + H)⁺ |
| | 3-yl)-5-(1H-tetrazol- | | | (d, J = 8.6 Hz, 1H), | | (ES⁺) at |
| | 5-yl)phenyl)amino)- | | | 7.29-7.37 (m, 1H), | | 3.93 min, |
| | 4-hydroxycyclobut- | | | 7.37-7.43 (m, 2H), | | UV active |
| | 3-ene-1,2-dione | | | 7.48-7.52 (m, 2H), | | |
| | | | | 7.70-7.74 (m, 1H), | | |
| | | | | 7.97-8.00 (m, 1H), | | |
| | | | | 8.12 (dd, J = 2.6, 8.6 | | |
| | | | | Hz, 1H), 8.36-8.46 (m, | | |
| | | | | 1H), 8.59 (d, J = 2.6 | | |
| | | | | Hz, 1H), 9.64 (s, 1H). | | |
| | | | | Two acidic protons not | | |
| | | | | observed. Ammonium | | |
| | | | | signal observed at | | |
| | | | | 6.95-7.20 ppm. | | |
| 17 | 3-hydroxy-4-((6- | 78 and 11 | Route E | (400 MHz, DMSO-d₆) | L | m/z 289 |
| | methoxy-4-(1H- | | | δ: 3.98 (s, 3H), 7.11 | | (M + H)⁺ |
| | tetrazol-5-yl)pyridin- | | | (d, J = 1.2 Hz, 1H), | | (ES⁺) at |
| | 2-yl)amino)cyclobut- | | | 7.56 (d, J = 1.2 Hz, | | 1.66 min, |
| | 3-ene-1,2-dione | | | 1H), 11.16 (s, 1H). | | UV active |
| | | | | Two acidic protons not | | |
| | | | | observed. | | |
| 18 | 3-hydroxy-4-((6- | 94 and 11 | Route E | (400 MHz, DMSO-d₆) | L | m/z 333 (M − |
| | phenyl-4-(1H- | | | δ: 7.51-7.69 (m, 3H), | | H)⁻ (ES⁻) at |
| | tetrazol-5-yl)pyridin- | | | 7.97 (d, J = 1.3 Hz, | | 1.89 min, |
| | 2-yl)amino)cyclobut- | | | 1H), 8.01-8.04 (m, | | UV active |
| | 3-ene-1,2-dione | | | 2H), 8.05 (d, J = 1.3 | | |
| | | | | Hz, 1H), 11.63 (s, 1H). | | |
| | | | | Two acidic protons not | | |
| | | | | observed. | | |
| 19 | 3-((3-(1H-tetrazol-5- | 31 and 95 | Route D | (400 MHz, DMSO-d₆) | K | m/z 532 |
| | yl)-5-(2-(4- | | | δ: 5.82 (s, 2H), 7.47- | | (M + H)⁺ |
| | (trifluoromethyl)benzyl)- | | | 7.56 (m, 2H), 7.68- | | (ES⁺) at |
| | 2H-indazol-5- | | | 7.79 (m, 4H), 7.81 (s, | | 4.42 min, |
| | yl)phenyl)amino)-4- | | | 1H), 8.01 (s, 1H), 8.13 | | UV active |
| | hydroxycyclobut-3- | | | (s, 1H), 8.42 (s, 1H), | | |
| | ene-1,2-dione | | | 8.69 (s, 1H), 9.64 (s, | | |
| | | | | 1H). Two acidic | | |
| | | | | protons not observed. | | |
| 20 | 3-((3-(1H-tetrazol-5- | 31 and 35 | Route D | (400 MHz, DMSO-d₆) | K | m/z 509 |
| | yl)-5-(6-((4- | | | δ: 5.55 (s, 2H), 7.06 | | (M + H)⁺ |
| | (trifluorometh- | | | (d, J = 8.7 Hz, 1H), | | (ES⁺) at |
| | yl)benzyl)oxy)pyridin- | | | 7.70-7.75 (m, 3H), | | 4.13 min, |
| | 3-yl)phenyl)amino)-4- | | | 7.77 (d, J = 8.3 Hz, | | UV active |
| | hydroxycyclobut-3- | | | 2H), 7.84-7.89 (m, | | |
| | ene-1,2-dione | | | 1H), 8.12 (dd, J = 2.5, | | |
| | | | | 8.7 Hz, 1H), 8.24-8.32 | | |
| | | | | (m, 1H), 8.55 (d, J = | | |
| | | | | 2.5 Hz, 1H), 9.45 (s, | | |
| | | | | 1H). Two acidic | | |
| | | | | protons not observed. | | |
| 21 | 3-((3-(2-benzyl-2H- | 31 and 40 | Route D | (400 MHz, DMSO-d₆) | K | m/z 464 |
| | indazol-5-yl)-5-(1H- | | | δ: 5.69 (s, 2H), 7.24- | | (M + H)⁺ |
| | tetrazol-5- | | | 7.59 (m, 5H), 7.69- | | (ES⁺) at |
| | yl)phenyl)amino)-4- | | | 7.74 (m, 2H), 7.79 (s, | | 3.62 min, |
| | hydroxycyclobut-3- | | | 1H), 7.98 (s, 1H), 8.10 | | UV active |
| | ene-1,2-dione | | | (s, 1H), 8.38 (s, 1H), | | |
| | | | | 8.62 (s, 1H), 9.59 (s, | | |
| | | | | 1H). Two acidic | | |
| | | | | protons not observed. | | |
| | | | | Ammonium signal | | |
| | | | | observed at 6.95-7.20 | | |
| | | | | ppm. | | |
| 22 | 3-hydroxy-4-((6- | 61 and 11 | Route E | (400 MHz, DMSO-d₆) | M | m/z 349 (M − |
| | phenoxy-4-(1H- | | | δ: 6.91 (s, 1H), 7.24- | | H)⁻ (ES⁻) |
| | tetrazol-5-yl)pyridin- | | | 7.35 (m, 3H), 7.45- | | 1.92 min, |
| | 2-yl)amino)cyclobut- | | | 7.52 (m, 2H), 7.96 (s, | | UV active |
| | 3-ene-1,2-dione | | | 1H), 10.61 (s, 1H). | | |
| | | | | Two acidic protons not | | |
| | | | | observed. Ammonium | | |
| | | | | signal observed at | | |
| | | | | 6.95-7.20 ppm. | | |
| 23 | 3-((3-(1H-tetrazol-5- | 31 and 96 | Route D | (400 MHz, DMSO-d₆) | K | m/z 562 |
| | yl)-5-(2-(2-(4- | | | δ: 4.64 (t, J = 5.1 Hz, | | (M + H)⁺ |
| | (trifluorometh- | | | 2H), 4.89 (t, J = 5.1 | | (ES⁺) at |
| | yl)phenoxy)ethyl)- | | | Hz, 2H), 7.16 (d, J = | | 3.94 min, |
| | 2H-indazol-6- | | | 8.6 Hz, 2H), 7.64 (d, J = | | UV active |
| | yl)phenyl)amino)-4- | | | 8.6 Hz, 2H), 7.69- | | |
| | hydroxycyclobut-3- | | | 7.71 (m, 2H), 7.79- | | |
| | ene-1,2-dione | | | 7.83 (m, 1H), 7.82- | | |
| | | | | 7.87 (m, 1H), 8.04- | | |
| | | | | 8.08 (m, 1H), 8.24- | | |
| | | | | 8.32 (m, 1H), 8.56 (s, | | |
| | | | | 1H), 9.40 (s, 1H). Two | | |
| | | | | acidic protons not | | |
| | | | | observed. | | |
| 24 | 3-((3-cyclopropyl-5- | 21 and 11 | Route A, | (400 MHz, DMSO-d₆) | K | m/z 298 |
| | (1H-tetrazol-5- | | steps (ii)- | δ: 0.74 (s, 2H), 0.95 | | (M + H)⁺ |
| | yl)phenyl)amino)-4- | | (iii) | (s, 2H), 1.88 (s, 1H), | | (ES⁺) at |
| | hydroxycyclobut-3- | | | 7.21 (s, 1H), 7.61 (s, | | 2.95 min, |
| | ene-1,2-dione | | | 1H), 7.78 (s, 1H), 9.36 | | UV active |
| | | | | (m, 1H). Two acidic | | |
| | | | | protons not observed. | | |
| | | | | Ammonium signal | | |
| | | | | observed at 6.95-7.20 | | |
| | | | | ppm. | | |
| 25 | 3-((3-(1H-indazol-5- | 31 and 97 | Route D | (400 MHz, DMSO-d₆) | K | m/z 372 (M − |
| | yl)-5-(1H-tetrazol-5- | | | δ: 7.66 (d, J = 8.9 Hz, | | H)⁻ (ES⁻) at |
| | yl)phenyl)amino)-4- | | | 1H), 7.76-7.87 (m, | | 1.47 min, |
| | hydroxycyclobut-3- | | | 2H), 7.96 (s, 1H), | | UV active |
| | ene-1,2-dione | | | 8.08-8.23 (m, 2H), | | |
| | | | | 8.38 (s, 1H), 9.57 (s, | | |
| | | | | 1H), 13.16 (s, 1H). | | |
| | | | | Two acidic protons not | | |
| | | | | observed. Ammonium | | |
| | | | | signal observed at | | |
| | | | | 6.95-7.20 ppm. | | |
| 26 | 3-((3-(6-(2-(3,5- | 31 and 98 | Route D | (400 MHz, DMSO-d₆) | K | m/z 607 |
| | bis(trifluorometh- | | | δ: 4.55-4.64 (m, 2H), | | (M + H)⁺ |
| | yl)phenoxy)ethoxy)pyridin- | | | 4.67-4.79 (m, 2H), | | (ES⁺) at |
| | 3-yl)-5-(1H-tetrazol-5- | | | 7.01 (d, J = 8.6 Hz, | | 2.13 min, |
| | yl)phenyl)amino)-4- | | | 1H), 7.66 (s, 1H), 7.72 | | UV active |
| | hydroxycyclobut-3- | | | (s, 3H), 8.00 (s, 1H), | | |
| | ene-1,2-dione | | | 8.13 (dd, J = 2.6, 8.6 | | |
| | | | | Hz, 1H), 8.45 (s, 1H), | | |
| | | | | 8.59-8.62 (m, 1H), | | |
| | | | | 9.66 (s, 1H). Two | | |
| | | | | acidic protons not | | |
| | | | | observed. Ammonium | | |
| | | | | signal observed at | | |
| | | | | 6.95-7.20 ppm. | | |
| 27 | 3-((3-(1H-tetrazol-5- | 31 and 99 | Route D | (400 MHz, DMSO-d₆) | K | m/z 539 |
| | yl)-5-(6-(2-(4- | | | δ: 4.45-4.49 (m, 2H), | | (M + H)⁺ |
| | (trifluorometh- | | | 4.67-4.73 (m, 2H), | | (ES⁺) at |
| | yl)phenoxy)ethoxy)pyridin- | | | 7.03 (d, J = 8.6 Hz, | | 4.12 min, |
| | 3-yl)phenyl)amino)- | | | 1H), 7.21 (d, J = 8.4 | | UV active |
| | 4-hydroxycyclobut- | | | Hz, 2H), 7.68 (d, J = | | |
| | 3-ene-1,2-dione | | | 8.4 Hz, 2H), 7.73 (s, | | |
| | | | | 1H), 7.99 (s, 1H), | | |
| | | | | 8.10-8.14 (m, 1H), | | |
| | | | | 8.43 (s, 1H), 8.60 (s, | | |
| | | | | 1H), 9.66 (s, 1H). Two | | |
| | | | | acidic protons not | | |
| | | | | observed. | | |
| 28 | 3-((3-cyclohexyl-5- | 101 and 11 | Route A, | (400 MHz, DMSO-d₆) | K | m/z 357 |
| | (1H-tetrazol-5- | | steps (ii)- | δ: 1.19-1.30 (m, 1H), | | (M + Na)⁺ |
| | yl)phenyl)amino)-4- | | (iii) | 1.33-1.50 (m, 4H), | | (ES⁺) at |
| | hydroxycyclobut-3- | | | 1.69-1.76 (m, 1H), | | 1.43 min, |
| | ene-1,2-dione | | | 1.78-1.83 (m, 4H), | | UV active |
| | | | | 2.41-2.47 (m, 1H), | | |
| | | | | 7.31-7.34 (m, 1H), | | |
| | | | | 7.72-7.77 (m, 1H), | | |
| | | | | 7.79-7.84 (m, 1H), | | |
| | | | | 9.35 (s, 1H). Two | | |
| | | | | acidic protons not | | |
| | | | | observed. Ammonium | | |
| | | | | signal observed at | | |
| | | | | 6.95-7.20 ppm. | | |
| 29 | 3-hydroxy-4-((3- | 102 and 11 | Route A, | (400 MHz, D₂O) δ: | K | m/z 336 |
| | (pyridazin-4-yl)-5- | | steps (ii)- | 7.59 (s, 1H), 7.70-7.86 | | (M + H)⁺ |
| | (1H-tetrazol-5- | | (iii) | (m, 3H), 8.94 (s, 1H), | | (ES⁺) at |
| | yl)phenyl)amino)cyclobut- | | | 9.25 (s, 1H). Three | | 2.73 min, |
| | 3-ene-1,2-dione | | | exchangeable protons | | UV active |
| | | | | not observed. | | |
| 30 | 3′-((2-hydroxy-3,4- | 1 and 80 | Route F | (400 MHz, DMSO-d₆) | J | m/z 405 |
| | dioxocyclobut-1-en- | | | δ: 3.01 (s, 6H), 7.54- | | (M + H)⁺ |
| | 1-yl)amino)-N,N- | | | 7.56 (d, J = 8.0 Hz, | | (ES⁺) at |
| | dimethyl-5′-(1H- | | | 1H), 7.83-7.93 (m, | | 1.69 min, |
| | tetrazol-5-yl)-[1,1′- | | | 3H), 8.07-8.13 (m, | | UV active |
| | biphenyl]-4- | | | 2H), 8.21-8.24 (m, | | |
| | carboxamide | | | 1H), 9.93 (s, 1H). Two | | |
| | | | | acidic protons not | | |
| | | | | observed. | | |
| 31 | N-(4- | 1 and 54 | Route F | (400 MHz, DMSO-d₆) | I | m/z 508 (M − |
| | carbamoylbenzyl)- | | | δ: 4.57-4.59 (m, 2H), | | H)⁻ (ES⁻) at |
| | 3′-((2-hydroxy-3,4- | | | 7.35-7.43 (m, 2H), | | 1.71 min, |
| | dioxocyclobut-1-en- | | | 7.74-7.94 (m, 5H), | | UV active |
| | 1-yl)amino)-5′-(1H- | | | 8.01-8.25 (m, 4H), | | |
| | tetrazol-5-yl)-[1,1′- | | | 8.98-8.99 (m, 1H), | | |
| | biphenyl]-4- | | | 10.10 (s, 1H). Two | | |
| | carboxamide | | | acidic protons not | | |
| | | | | observed and two | | |
| | | | | primary carboxamide | | |
| | | | | protons not observed. | | |

### Crystalline Compounds

### Preparation of Compound a Tromethamine Salt (Hydrate 1) (Un-Seeded)

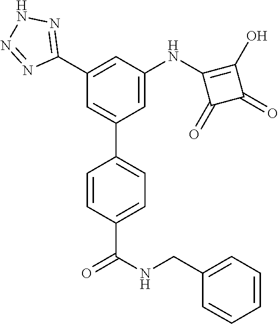 [SMILES: O=C(NCc1ccccc1)c1ccc(-c2cc(Nc3c(O)c(=O)c3=O)cc(-c3nn[nH]n3)c2)cc1]

Compound A is the compound of Example 5 (structure above). 2 mL of 97.6% (THF:water 3:1): 2.4% DMSO were added to 50 mg of Compound A and the mixture was heated to 50° C. resulting in dissolution. 1 eq of 1 M aqueous tromethamine was added to the solution and it was equilibrated at 50° C. for one hour before being cooled to room temperature and left stirring overnight. The solution was then evaporated (to approx. 25% of original volume) under nitrogen until precipitation occurred. The resulting solid was filtered, washed with IPA and dried under vacuum at 45° C.

### Preparation of Compound a Tromethamine Salt (Hydrate 1) (Seeded)

Compound A (1 wt, kg scale) was charged to a vessel under nitrogen. This was followed by the addition of THF (6.08 vol) and then aqueous tromethamine solution (0.268 wt tromethamine dissolved in 2.21 vol of water). Water (1.84 vol) was then added to the vessel. The mixture was heated to dissolution at 60° C. The solution was cooled to 50° C. Acetonitrile (1.29 vol) was added before the reaction was seeded with crystalline Hydrate I of Compound A tromethamine salt (0.0126 wt). The mixture was left to equilibrate for 1 hour. Acetonitrile (6.33 vol) was then added to the slurry over 2 hours. The mixture was then cooled to 5° C. and stirred overnight. The solids were washed with acetonitrile (3.96 vol) before being dried under vacuum at 45° C. to give Compound A tromethamine salt (Hydrate I).

### Properties of Compound a Tromethamine Salt (Hydrate 1)

The X-ray powder diffraction (XRPD) pattern of the above Hydrate I of Compound A tromethamine salt is shown in FIG. 1 and a summary of the diffraction angle and d-spacings are given in Table 4 (characteristic peaks) and Table 5 (complete peak list). The XRPD analysis was conducted on a PANalytical Xpert Pro diffractometer on Si zero-background wafers. The acquisition conditions included Cu K_{α} radiation, generator tension: 40 kV, generator current: 45 mA, step size 0.02° 2θ, start angle: 2.0° 2θ, end angle: 40.0° 2θ.

TABLE 4

| Pos. [°2θ] | d-spacings [Å] |
|---|---|
| 3.9 ± 0.2 | 22.8 |
| 7.7 ± 0.2 | 11.4 |
| 10.0 ± 0.2 | 8.8 |
| 15.8 ± 0.2 | 5.6 |

TABLE 5

| Pos. [°2θ] | d-spacings [Å] |
|---|---|
| 3.9 ± 0.2 | 22.8 |
| 7.7 ± 0.2 | 11.4 |
| 10.0 ± 0.2 | 8.8 |
| 11.6 ± 0.2 | 7.6 |
| 12.4 ± 0.2 | 7.1 |
| 13.0 ± 0.2 | 6.8 |
| 13.6 ± 0.2 | 6.5 |
| 14.7 ± 0.2 | 6.0 |
| 14.8 ± 0.2 | 6.0 |
| 15.8 ± 0.2 | 5.6 |
| 16.7 ± 0.2 | 5.3 |
| 17.2 ± 0.2 | 5.2 |
| 18.0 ± 0.2 | 4.9 |
| 18.4 ± 0.2 | 4.8 |
| 18.9 ± 0.2 | 4.7 |
| 19.7 ± 0.2 | 4.5 |
| 20.1 ± 0.2 | 4.4 |
| 20.6 ± 0.2 | 4.3 |
| 21.3 ± 0.2 | 4.2 |
| 21.9 ± 0.2 | 4.1 |
| 22.6 ± 0.2 | 3.9 |
| 23.3 ± 0.2 | 3.8 |
| 24.7 ± 0.2 | 3.6 |
| 24.8 ± 0.2 | 3.6 |
| 25.4 ± 0.2 | 3.5 |
| 25.7 ± 0.2 | 3.5 |
| 26.5 ± 0.2 | 3.4 |
| 26.9 ± 0.2 | 3.3 |
| 27.2 ± 0.2 | 3.3 |
| 28.4 ± 0.2 | 3.1 |
| 29.2 ± 0.2 | 3.1 |
| 29.9 ± 0.2 | 3 |
| 31.0 ± 0.2 | 2.9 |
| 32.0 ± 0.2 | 2.8 |
| 33.1 ± 0.2 | 2.7 |
| 35.8 ± 0.2 | 2.5 |
| 40.0 ± 0.2 | 2.3 |

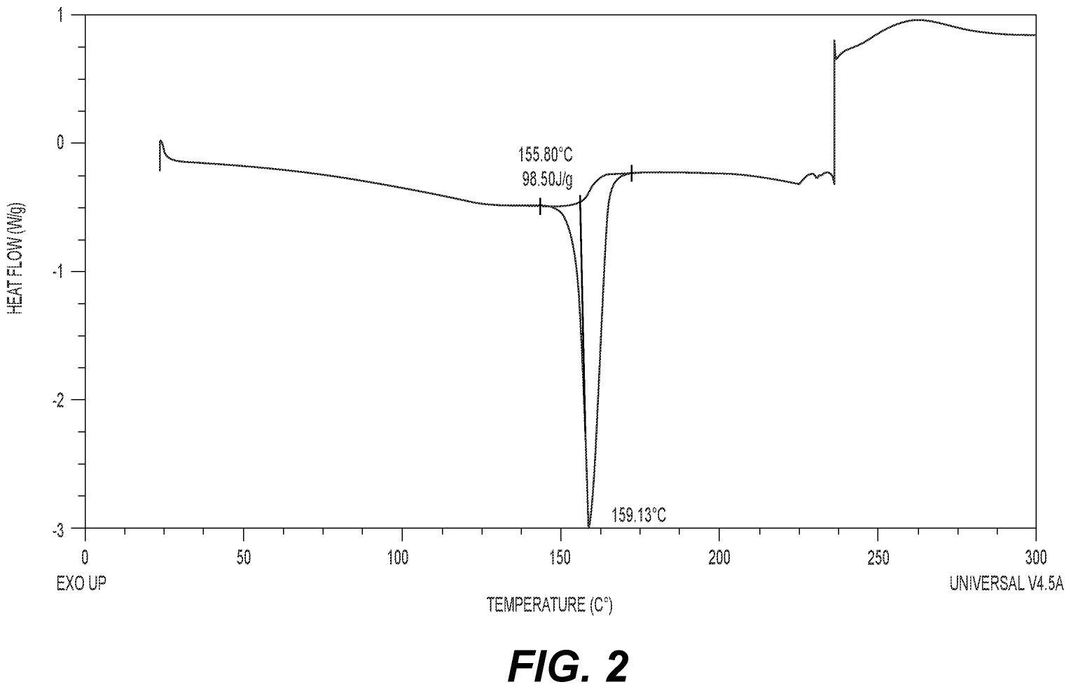

An example differential scanning calorimetry (DSC) thermogram of the above Hydrate I of Compound A tromethamine salt is shown in FIG. 2. The DSC analysis was conducted with a TA Instruments Q100 differential scanning calorimeter equipped with an autosampler and a refrigerated cooling system under 50 mL/min N₂ purge. DSC thermograms of samples were obtained at 10° C./min in a crimped Al pan. The DSC thermogram exhibits an endotherm with an onset temperature of about 155° C. However, this may vary depending on the experimental conditions and level of crystallinity.

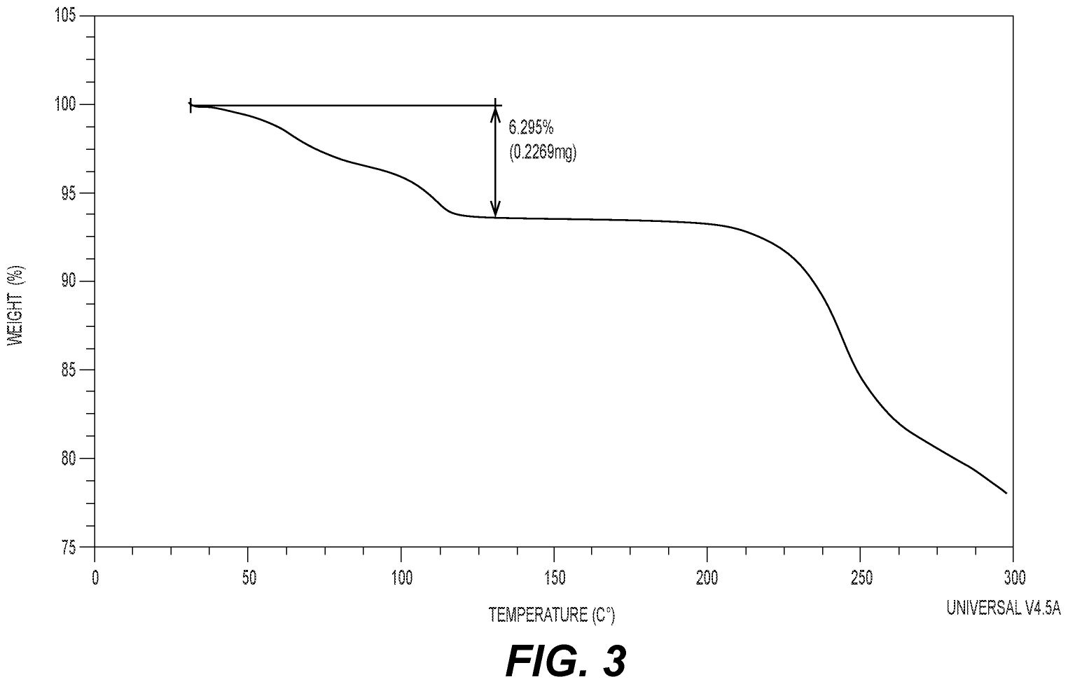

An example thermogravimetric analysis (TGA) thermogram of the above Hydrate I of Compound A tromethamine salt is shown in FIG. 3. The TGA analysis was conducted on a TA Instruments Q5000 thermogravimetric analyzer under 25 mL/min N₂ flow and a heating rate of 10° C./min. The TGA thermogram of this hydrate typically exhibits a weight loss of between 6-7% from 30-120° C., which corresponds to about 2-2.5 equivalent of water for each equivalent of Compound A, i.e., a variable hydrate.

### Preparation of Compound a Tromethamine Salt (Hydrate II) (Un-Seeded)

2 mL of methanol were added to 100 mg of an amorphous form of Compound A tromethamine salt and the suspension was equilibrated at room temperature overnight. The resulting solid was filtered and dried under vacuum at 45° C.

### Preparation of Compound a Tromethamine Salt (Hydrate II) (Seeded)

60 mL of methanol were added to 3 g of Hydrate I of Compound A tromethamine salt in a vessel with overhead stirring. 60 mg (2% w/w seed loading) of the Hydrate II of Compound A tromethamine salt was added and the reaction mixture was equilibrated overnight at room temperature. The suspension thickened and became much paler overnight. The resulting solid was filtered and dried under vacuum at 45° C.

The X-ray powder diffraction (XRPD) pattern of the above Hydrate II of Compound A tromethamine salt is shown in FIG. 4 and a summary of the diffraction angle and d-spacings are given in Table 6 (characteristic peaks) and Table 7 (complete peak list). The XRPD analysis was conducted on a PANalytical Xpert Pro diffractometer on Si zero-background wafers. The acquisition conditions included Cu K_{α} radiation, generator tension: 45 kV, generator current: 40 mA, step size 0.03° 2θ, start angle: 3.0° 2θ, end angle: 35.0° 2θ.

TABLE 6

| Pos. [°2θ] | d-spacings [Å] |
|---|---|
| 4.4 ± 0.2 | 20.2 |
| 14.4 ± 0.2 | 6.1 |
| 23.9 ± 0.2 | 3.7 |

TABLE 7

| Pos. [°2θ] | d-spacings [Å] |
|---|---|
| 4.4 ± 0.2 | 20.2 |
| 5.4 ± 0.2 | 16.3 |
| 9.0 ± 0.2 | 9.8 |
| 11.1 ± 0.2 | 7.9 |
| 11.5 ± 0.2 | 7.7 |
| 11.7 ± 0.2 | 7.6 |
| 12.6 ± 0.2 | 7 |
| 13.4 ± 0.2 | 6.6 |
| 13.7 ± 0.2 | 6.5 |
| 14.4 ± 0.2 | 6.1 |
| 16.8 ± 0.2 | 5.3 |
| 18.0 ± 0.2 | 4.9 |
| 18.4 ± 0.2 | 4.8 |
| 18.9 ± 0.2 | 4.7 |
| 20.3 ± 0.2 | 4.4 |
| 21.1 ± 0.2 | 4.2 |
| 21.9 ± 0.2 | 4.1 |
| 22.4 ± 0.2 | 4 |
| 22.6 ± 0.2 | 3.9 |
| 23.0 ± 0.2 | 3.9 |
| 23.9 ± 0.2 | 3.7 |
| 25.7 ± 0.2 | 3.5 |
| 26.1 ± 0.2 | 3.4 |
| 26.8 ± 0.2 | 3.3 |
| 27.2 ± 0.2 | 3.3 |
| 27.9 ± 0.2 | 3.2 |
| 28.5 ± 0.2 | 3.1 |
| 29.2 ± 0.2 | 3.1 |
| 29.9 ± 0.2 | 3 |
| 30.4 ± 0.2 | 2.9 |
| 32.0 ± 0.2 | 2.8 |
| 32.8 ± 0.2 | 2.7 |
| 34.0 ± 0.2 | 2.6 |

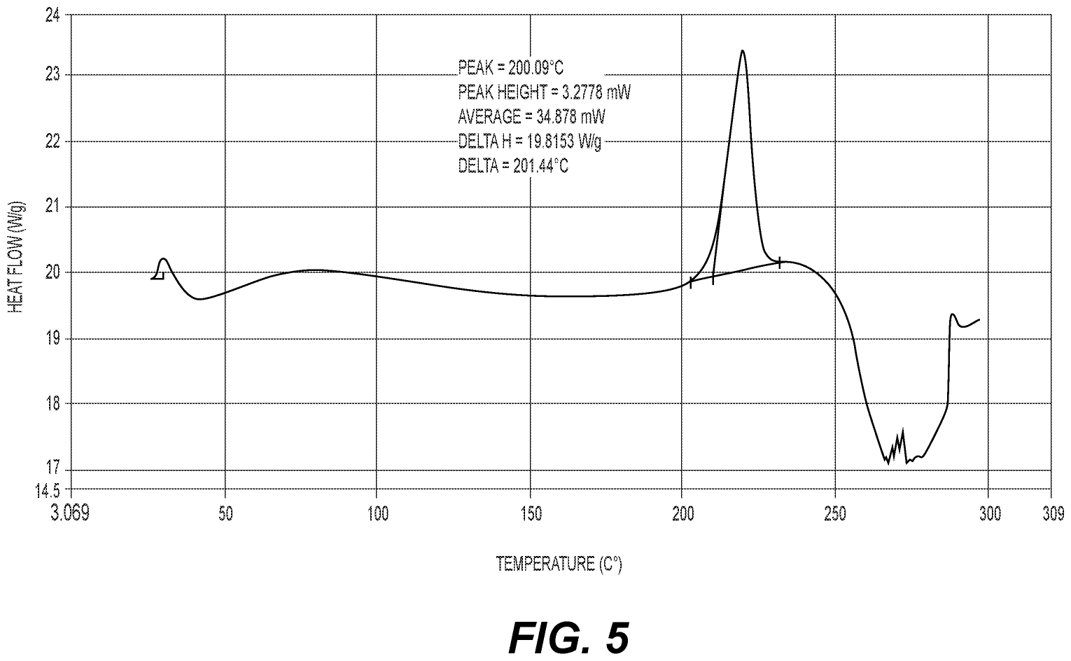

An example differential scanning calorimetry (DSC) thermogram of the above Hydrate II of Compound A tromethamine salt is shown in FIG. 5. The DSC analysis was conducted with a PerkinElmer Pyris 6000 differential scanning calorimeter equipped with an autosampler and a refrigerated cooling system under 20 mL/min N₂ purge. DSC thermograms of samples were obtained at 20° C./min in a pin hole Al pan. The DSC thermogram exhibits an endotherm with an onset temperature of about 210° C. However, this may vary depending on the experimental conditions and level of crystallinity.

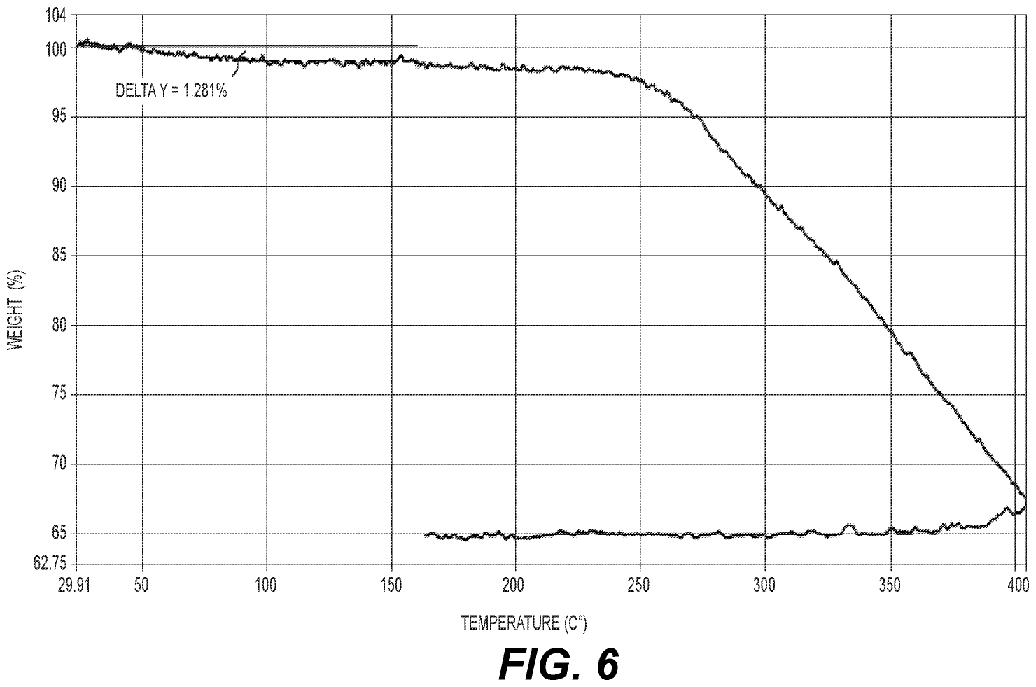

An example thermogravimetric analysis (TGA) thermogram of the above Hydrate II of Compound A tromethamine salt is shown in FIG. 6. The TGA analysis was conducted on a PerkinElmer Pyris 1 thermogravimetric analyzer under 20 mL/min N₂ flow and a heating rate of 20° C./min. The TGA thermogram of this hydrate typically exhibits a weight loss of between 1-3% from 30-150° C., which corresponds to about 0.3-1 equivalent of water for each equivalent of Compound A, i.e., a variable hydrate.

Water activity studies were conducted to determine the critical water activity at which each of Hydrate I and Hydrate II is stable. Competitive slurries of Hydrate I and Hydrate II were conducted at room temperature in a range of aqueous solvent mixtures with varying water activity. Hydrate I was isolated from all mixtures with a water activity of greater than 0.5. Equilibration of Hydrate II alone confirmed conversion to Hydrate I in solvent mixtures with water activity of 0.5 or greater. Competitive slurries of Hydrate I and Hydrate II at room temperature in solvent mixtures with a water activity of 0.2 resulted in a mixture of Hydrate I and Hydrate II solids.

### Preparation of Crystalline Compound a (Free Acid) (Pattern 3)

Crude Compound A (free acid) was purified by reverse phase chromatography applying basic conditions (high pH) under 5-35% gradient of acetonitrile in aqueous media (0.2% of 28% ammonia hydroxide in water) on 12.5 minute method via a Gemini-NX C18 column (5 μm, 100×30 mm) on a Gilson Semi Preparative HPLC, Pumps 332 & 331, GX-271 Liquid handler, Trilution software using a flow rate of 30 mL/min and 171 Diode Array Detector at 205 nm, 210 nm and 230 nm. The desired fractions were combined then evaporated on a Biotage V10 machine to give a white solid residue (4 g), a diammonium salt.

The diammonium salt (4 g, 7.99 mmol) was dissolved in DMSO (39.96 mL) and stirred for 30 minutes. 1 N HCl (59.94 mL, 59.94 mmol) was added and the resulting precipitate was collected by filtration, washed with ice cold water (20 mL) and dried to give crude product which was re-suspended in EtOH (40 mL) and stirred for 3 h. The suspension was then filtered to give a dry white solid which was milled to a fine powder (2.94 g). NMR revealed that this powder was the desired product; however a large amount of DMSO remained in the sample. Therefore, the solid was re-suspended in EtOH (25 mL) and stirred for 18 h. The suspension was then filtered, and the solid was collected, milled to a fine powder using a pestle and mortar to give Compound A free acid (2.39 g, 5.12 mmol, 64.1% yield) as a white crystalline solid. NMR of the product shows a small amount of DMSO remained in the sample with the ratio of product to DMSO being approximately 1:0.05. The X-ray powder diffraction (XRPD) pattern of the crystalline Compound A free acid is shown in FIG. 7, and this crystalline solid is referred to as “Pattern 3.”

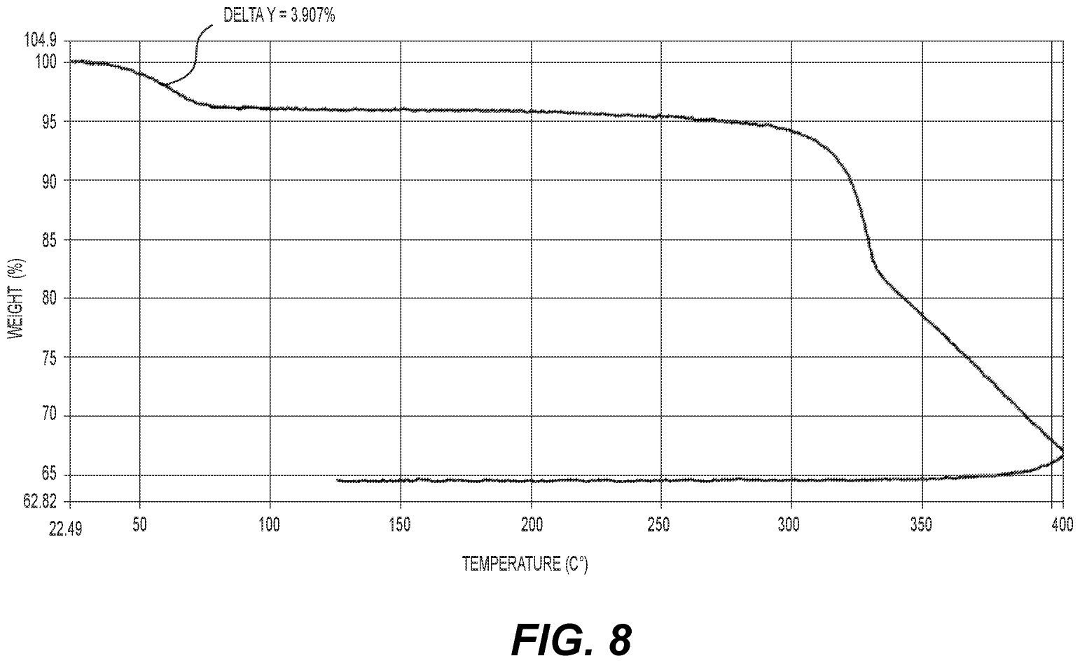

An example thermogravimetric analysis (TGA) thermogram of the above Compound A free acid Pattern 3 is shown in FIG. 8.

### Preparation of Crystalline Compound a (Free Acid) (Pattern 1)

When crystalline Compound A free acid Pattern 3 was dried by heating at 45° C. under vacuum (ca. 10-15 mbar) overnight, a different crystalline solid was obtained, referred to as “Pattern 1.” The XRPD pattern of Compound A free acid Pattern 1 is shown in FIG. 9.

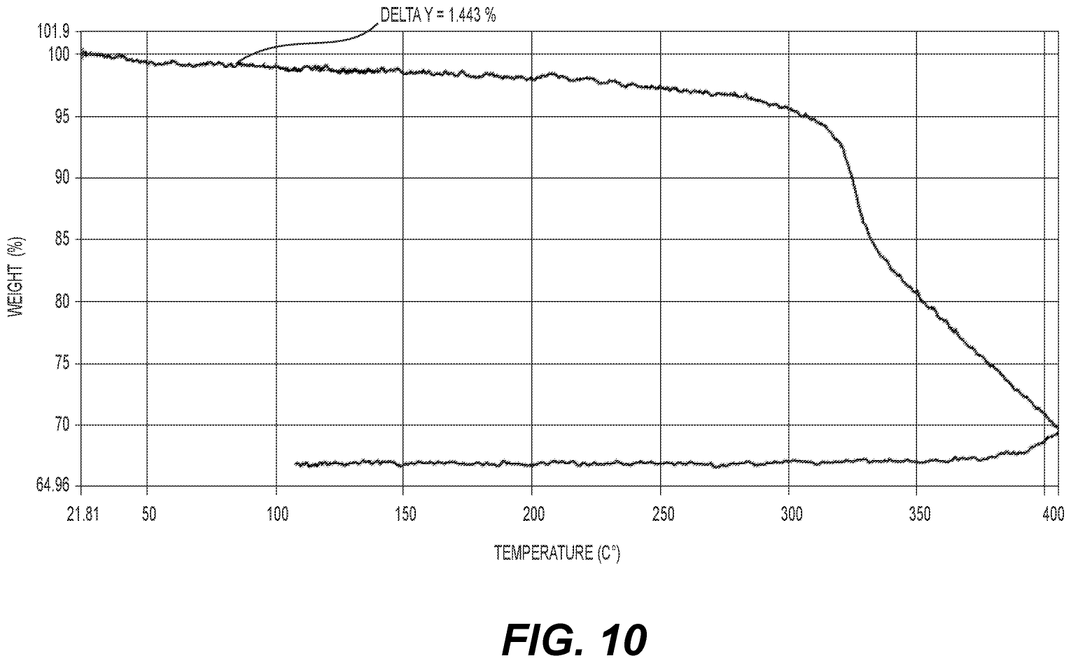

An example thermogravimetric analysis (TGA) thermogram of the above Compound A free acid Pattern 1 is shown in FIG. 10.

### Biological Activity

### Example A

### Human GPR35a Isoform Binding

Overexpression of Human GPR35a Baculovirus in HEK293f cells at a cell density of 2.5×10⁶ cells/mL and a multiplicity of infection of 2.5 over 24 h in Pro293 (Lonza)+5% FBS, 1% Glutamax and 0.4% Pen/Strep. Cells harvested and centrifuged at 2500 RPM for 10 mins at 4° C. The supernatant was then poured off and the pellet stored at −80° C. The pellet was defrosted and re-suspended in 15 mL of homogenising buffer (20 mM HEPES, 10 mM EDTA, pH 7.4). Then homogenised in mechanical homogeniser (VMR) for 10 seconds. The membrane was centrifuged in centrifuge tubes at 40,000 g for 15 mins at 4° C. The supernatant was poured away and re-suspended in 15 mL of homogenising buffer. Homogenised for 20 seconds. The membrane was centrifuged at 40,000 g for 45 mins at 4° C. The membrane was re-suspended in 3 mL of storage buffer (20 mM HEPES, 0.1 mM EDTA, pH 7.4) mixing well. The resulting membranes were then stored at −80°. GPR35 cell membrane homogenates were re-suspended in the binding buffer (50 mM TRIS+10 mM MgCl2 pH 7.4) to a final assay concentration of 5 ug/well. Test compounds were diluted in dimethylsulphoxide (DMSO (Sigma Aldrich, UK)), to form a 10 point ½ log concentration curve. Test compounds were added per plate, followed by 7 nM 3H-27966. 0.1 uM FAC Lodoxamide was added in order to allow non-specific binding to be calculated. Finally, membrane was added to each well on the plate. After 60 min incubation at room temperature, membranes were filtered onto a unifilter, 96-well white microplate with bonded GF/B filter, pre-soaked in ddH20, with a TomTec cell harvester, and washed 5 times with distilled water. Plates were dried prior to 50 ul/well scintillant added, sealed and radioactivity measured using a MicroBeta analyser. IC50 values were derived from the inhibition curve and affinity constant (Ki) values were calculated using the Cheng-Prussoff equation, where; pKi=−log 10 Ki.

### Label-Free DMR Functional Assay

HT-29 cells (ATCC HTB-38) kept in continuous culture in McCoys (Thermo 16600082) supplemented with 10% FBS. Day prior to assay, cells harvested with TrypLE (Gibco12604-013), and plated at 20 k/well in culture media in a total volume of 50 ul in Corning EPIC 384 well plates (5040) overnight 37° C. 5% CO₂. On the day of assay, cell media was removed and replaced with assay buffer (HBSS+20 mM HEPES pH7.4) and reincubated for 1 h. Compounds were prepared in 100% DMSO in ECHO LDV 384 source plates. Cell plates were read on an EPIC plate reader at room temperature for 15 mins, read paused, and cell plate added to LabCyte ECHO 550 for addition of 50 nl per well compound by acoustic transfer. Plates were immediately placed back into the EPIC reader, read unpaused and Dynamic Mass Redistribution measured for 60 minutes. Raw data was analysed by EPIC Analyser software and max peak taken per concentration to enable EC50 determination. All raw DMR data was normalised to Lodoxamide and buffer corrected.

TABLE 8
GPR35 Activity and HT-29 Affinity

| Example | Human GPR35 Ki [nM] | HT-29 EC50 [nM] |
|---|---|---|
| 1 | 9.81 | 8.68 |
| 2 | 0.48 | 0.90 |
| 3 | 0.35 | 2.27 |
| 4 | 3.83 | 2.50 |
| 5 | 0.08 | 0.52 |
| 6 | 1.82 | 0.62 |
| 7 | 2.77 | 4.20 |
| 8 | 1.47 | 0.66 |
| 9 | 0.46 | 0.83 |
| 10 | 2.75 | 2.34 |
| 11 | 6.24 | 4.31 |
| 12 | 0.31 | 0.03 |
| 13 | 0.99 | 2.39 |
| 14 | 0.60 | 4.90 |
| 15 | 0.16 | 2.02 |
| 16 | 0.06 | 0.27 |
| 17 | 7.85 | 9.25 |
| 18 | 2.65 | 5.18 |
| 19 | 0.36 | 0.08 |
| 20 | 1.06 | 7.68 |
| 21 | 0.91 | 0.14 |
| 22 | 3.66 | 1.39 |
| 23 | 0.46 | 0.12 |
| 24 | 15.21 | 8.19 |
| 25 | 0.37 | 0.44 |
| 26 | 0.36 | 1.04 |
| 27 | 0.75 | 0.54 |
| 28 | 12.50 | 6.65 |
| 29 | 0.74 | 1.05 |
| 30 | 5.99 | 2.64 |
| 31 | 1.61 | 11.31 |

Ki values derived from concentration response analysis in HEKf cell membranes overexpressing GPR35a isoform. EC50 values derived from concentration response analysis in HT-29 cell line endogenously expressing human GPR35.

Compound A showed about 500 times higher functional potency for GPR35 than the mast cell stabiliser Cromolyn, which has been used clinically at high doses for GI disorders. Compound A also showed pharmacology across preclinical species including in PGE2-induced fluid secretion, indomethacin ileitis and barrier permeability, TNBS mouse visceral pain model and acute rat and mouse LPS challenge. Compound A further showed strong selectivity for GPR35 and no off-target effects have been observed. Compound A has a very low drug interaction potential for the major human CYPs, including CYP3A4.

1. A compound of formula (1): 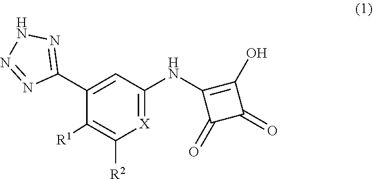
or a salt or tautomer thereof, wherein;
X is N or CH;
R¹ is H or halo;
R² is H, halo, optionally substituted C1-6 alkyl, optionally substituted C3-6 cycloalkyl, optionally substituted C1-6 alkoxy, optionally substituted aryl, optionally substituted heteroaryl or optionally substituted O-aryl.

2. The compound according to claim 1, or a salt or tautomer thereof, wherein X is CH.

3. The compound according to claim 1, or a salt or tautomer thereof, wherein X is N.

4. (canceled)

5. (canceled)

6. The compound according to claim 1, which is a compound of formula (2a) or (2b): 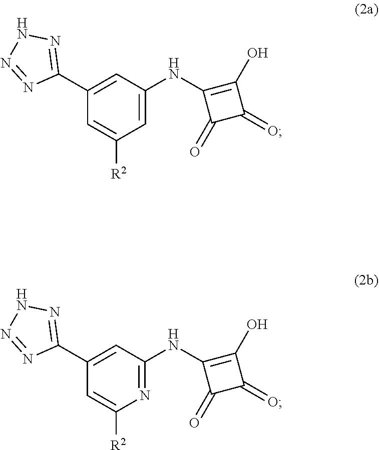
or a salt or tautomer thereof.

7. The compound according to claim 1, or a salt or tautomer thereof, wherein R² is H, C₁₋₆ alkyl optionally substituted with 1 to 6 fluorine atoms, C₃₋₆ cycloalkyl optionally substituted with 1 to 6 fluorine atoms or C₁₋₆ alkoxy optionally substituted with 1 to 6 fluorine atoms.

8. The compound according to claim 7, or a salt or tautomer thereof, wherein R² is H, trifluoromethyl, ethyl, cyclopropyl, cyclohexyl or methoxy.

9. The compound according to claim 1, or a salt or tautomer thereof, wherein R² is phenyl optionally substituted with R³, pyridyl optionally substituted with R³, O-phenyl optionally substituted with R³, indazolyl optionally substituted with R³ or pyridazinyl optionally substituted with R³, wherein R³ is H, halo, C₁₋₆ alkyl optionally substituted with 1 to 6 fluorine atoms, C₃₋₆ cycloalkyl optionally substituted with 1 to 6 fluorine atoms, C₁₋₆ alkoxy optionally substituted with 1 to 6 fluorine atoms, —CO₂R⁴, —CONHCH₂R⁴, —CONHCH₂CH₂OR⁴, —OR⁴, —OCH₂R⁴, —CH₂R⁴, —OCH₂R⁴, —CH₂CH₂OR⁴, —OCH₂CH₂OR⁴, —CONHR⁴ or —CON(CH₃)R⁴;
where R⁴ is H, C₁₋₆ alkyl optionally substituted with 1 to 6 fluorine atoms, or a group: 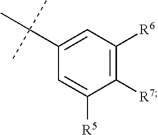
R⁵, R⁶ and R⁷ are independently H, halo, CO₂R⁸, CONR⁸R⁹ or C₁₋₆ alkyl optionally substituted with 1 to 6 fluorine atoms, wherein one or two carbon atoms of the C₁₋₆ alkyl group may be optionally replaced by O;
R⁸ and R⁹ are independently H or C₁₋₆ alkyl optionally substituted with 1 to 6 fluorine atoms.

10. The compound according to claim 9, wherein R³ is OMe, CO₂H, CO₂Et, CON(CH₃)₂, CONHCH₂CH₂OCH₃, or is selected from the group consisting of: 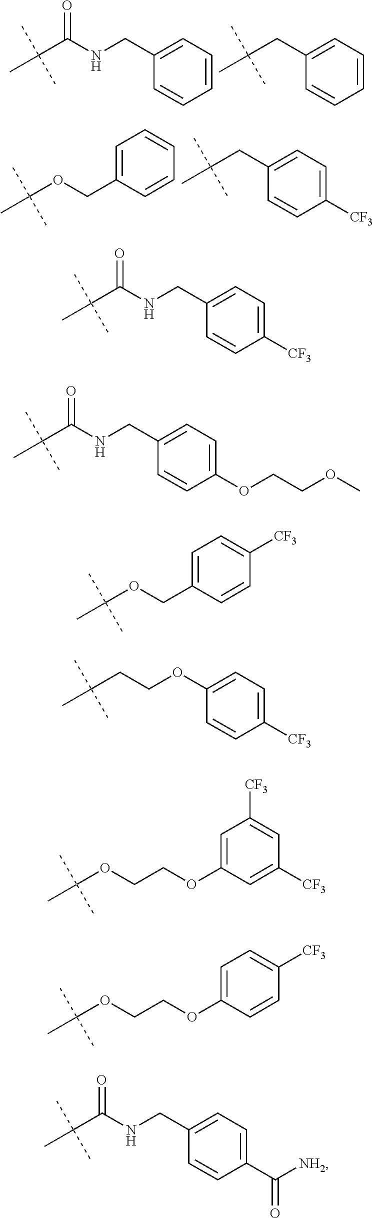
or a salt or tautomer thereof.

11. (canceled)

12. The compound according to claim 9, which is a compound of formula (4a), (4b), (4c), (4d) or (4e): 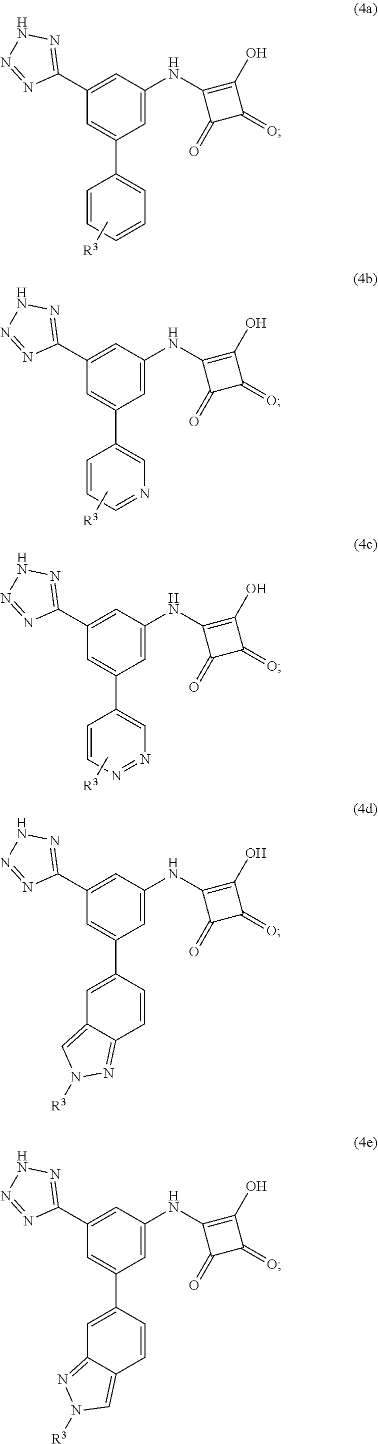
or a salt or tautomer thereof.

13. The compound according to claim 1, wherein R² is selected from the group consisting of: 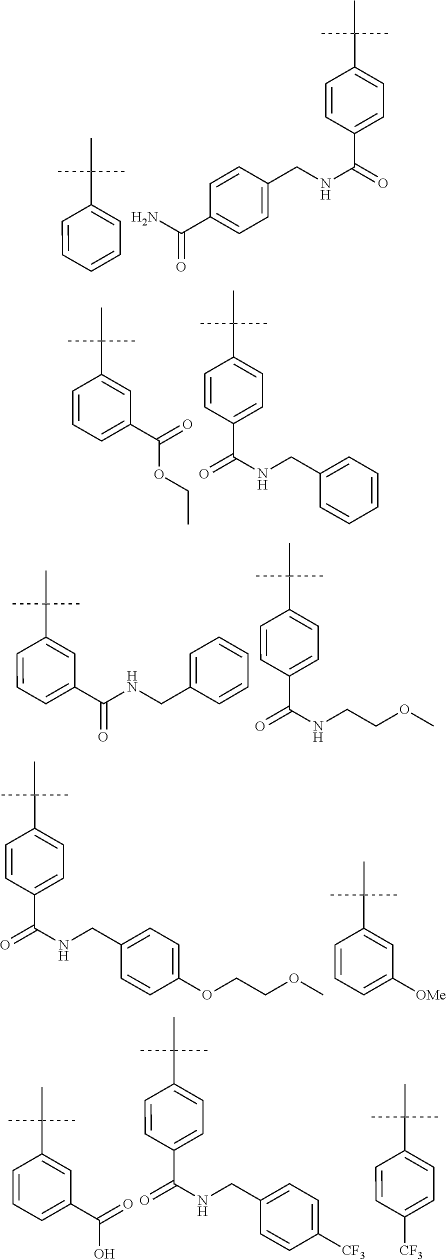 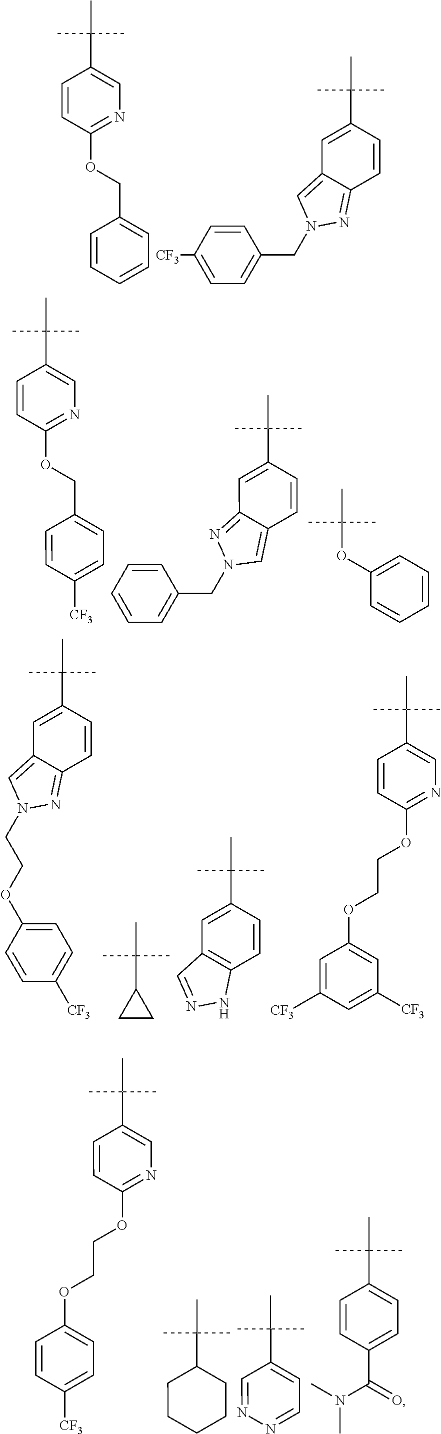
or a salt or tautomer thereof.

14. The compound according to claim 1, which is selected from the group consisting of:
3-((3-(1H-tetrazol-5-yl)phenyl)amino)-4-hydroxycyclobut-3-ene-1,2-dione;
3-((3-(1H-tetrazol-5-yl)-5-(trifluoromethyl)phenyl)amino)-4-hydroxycyclobut-3-ene-1,2-dione;
3-((5-(1H-tetrazol-5-yl)-[1,1′-biphenyl]-3-yl)amino)-4-hydroxycyclobut-3-ene-1,2-dione;
ethyl 3′-((2-hydroxy-3,4-dioxocyclobut-1-en-1-yl)amino)-5′-(1H-tetrazol-5-yl)-[1,1′-biphenyl]-3-carboxylate;
N-benzyl-3′-((2-hydroxy-3,4-dioxocyclobut-1-en-1-yl)amino)-5′-(1H-tetrazol-5-yl)-[1,1′-biphenyl]-4-carboxamide;
N-benzyl-3′-((2-hydroxy-3,4-dioxocyclobut-1-en-1-yl)amino)-5′-(1H-tetrazol-5-yl)-[1,1′-biphenyl]-3-carboxamide;
3′-((2-hydroxy-3,4-dioxocyclobut-1-en-1-yl)amino)-N-(2-methoxyethyl)-5′-(1H-tetrazol-5-yl)-[1,1′-biphenyl]-4-carboxamide;
3′-((2-hydroxy-3,4-dioxocyclobut-1-en-1-yl)amino)-N-(4-(2-methoxyethoxy)benzyl)-5′-(1H-tetrazol-5-yl)-[1,1′-biphenyl]-4-carboxamide;
3-hydroxy-4-((3′-methoxy-5-(1H-tetrazol-5-yl)-[1,1′-biphenyl]-3-yl)amino)cyclobut-3-ene-1,2-dione;
3-((3-ethyl-5-(1H-tetrazol-5-yl)phenyl)amino)-4-hydroxycyclobut-3-ene-1,2-dione;
3′-((2-hydroxy-3,4-dioxocyclobut-1-en-1-yl)amino)-5′-(1H-tetrazol-5-yl)-[1,1′-biphenyl]-3-carboxylic acid;
3′-((2-hydroxy-3,4-dioxocyclobut-1-en-1-yl)amino)-5′-(1H-tetrazol-5-yl)-N-(4-(trifluoromethyl)benzyl)-[1,1′-biphenyl]-4-carboxamide;
3-((4-fluoro-3-(1H-tetrazol-5-yl)phenyl)amino)-4-hydroxycyclobut-3-ene-1,2-dione;
3-((4-chloro-3-(1H-tetrazol-5-yl)phenyl)amino)-4-hydroxycyclobut-3-ene-1,2-dione;
3-((5-(1H-tetrazol-5-yl)-4′-(trifluoromethyl)-[1,1′-biphenyl]-3-yl)amino)-4-hydroxycyclobut-3-ene-1,2-dione;
3-((3-(6-(benzyloxy)pyridin-3-yl)-5-(1H-tetrazol-5-yl)phenyl)amino)-4-hydroxycyclobut-3-ene-1,2-dione;
3-hydroxy-4-((6-methoxy-4-(1H-tetrazol-5-yl)pyridin-2-yl)amino)cyclobut-3-ene-1,2-dione;
3-hydroxy-4-((6-phenyl-4-(1H-tetrazol-5-yl)pyridin-2-yl)amino)cyclobut-3-ene-1,2-dione;
3-((3-(1H-tetrazol-5-yl)-5-(2-(4-(trifluoromethyl)benzyl)-2H-indazol-5-yl)phenyl)amino)-4-hydroxycyclobut-3-ene-1,2-dione;
3-((3-(1H-tetrazol-5-yl)-5-(6-((4-(trifluoromethyl)benzyl)oxy)pyridin-3-yl)phenyl)amino)-4-hydroxycyclobut-3-ene-1,2-dione;
3-((3-(2-benzyl-2H-indazol-5-yl)-5-(1H-tetrazol-5-yl)phenyl)amino)-4-hydroxycyclobut-3-ene-1,2-dione;
3-hydroxy-4-((6-phenoxy-4-(1H-tetrazol-5-yl)pyridin-2-yl)amino)cyclobut-3-ene-1,2-dione;
3-((3-(1H-tetrazol-5-yl)-5-(2-(2-(4-(trifluoromethyl)phenoxy)ethyl)-2H-indazol-6-yl)phenyl)amino)-4-hydroxycyclobut-3-ene-1,2-dione;
3-((3-cyclopropyl-5-(1H-tetrazol-5-yl)phenyl)amino)-4-hydroxycyclobut-3-ene-1,2-dione;
3-((3-(1H-indazol-5-yl)-5-(1H-tetrazol-5-yl)phenyl)amino)-4-hydroxycyclobut-3-ene-1,2-dione;
3-((3-(6-(2-(3,5-bis(trifluoromethyl)phenoxy)ethoxy)pyridin-3-yl)-5-(1H-tetrazol-5-yl)phenyl)amino)-4-hydroxycyclobut-3-ene-1,2-dione;
3-((3-(1H-tetrazol-5-yl)-5-(6-(2-(4-(trifluoromethyl)phenoxy)ethoxy)pyridin-3-yl)phenyl)amino)-4-hydroxycyclobut-3-ene-1,2-dione;
3-((3-cyclohexyl-5-(1H-tetrazol-5-yl)phenyl)amino)-4-hydroxycyclobut-3-ene-1,2-dione;
3-hydroxy-4-((3-(pyridazin-4-yl)-5-(1H-tetrazol-5-yl)phenyl)amino)cyclobut-3-ene-1,2-dione;
3′-((2-hydroxy-3,4-dioxocyclobut-1-en-1-yl)amino)-N,N-dimethyl-5′-(1H-tetrazol-5-yl)-[1,1′-biphenyl]-4-carboxamide;
N-(4-carbamoylbenzyl)-3′-((2-hydroxy-3,4-dioxocyclobut-1-en-1-yl)amino)-5′-(1H-tetrazol-5-yl)-[1,1′-biphenyl]-4-carboxamide;
or a salt or tautomer thereof.

15. The salt according to claim 1, which is a pharmaceutically acceptable salt.

16. The compound or salt according to claim 1, wherein the compound is 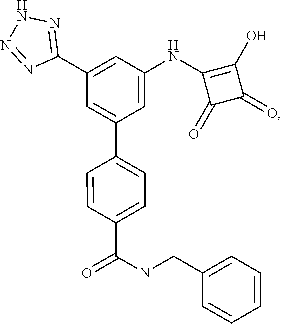 [SMILES: O=C(NCc1ccccc1)c1ccc(-c2cc(Nc3c(O)c(=O)c3=O)cc(-c3nn[nH]n3)c2)cc1]
or a pharmaceutically acceptable salt thereof.

17. The compound according to claim 1, wherein the compound is

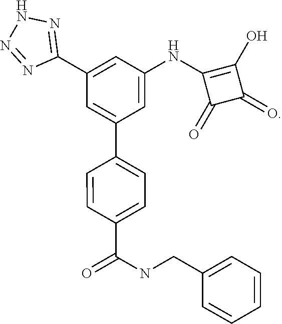 [SMILES: O=C(NCc1ccccc1)c1ccc(-c2cc(Nc3c(O)c(=O)c3=O)cc(-c3nn[nH]n3)c2)cc1]

18. The pharmaceutically acceptable salt according to claim 16, which is a tromethamine salt having the structure:

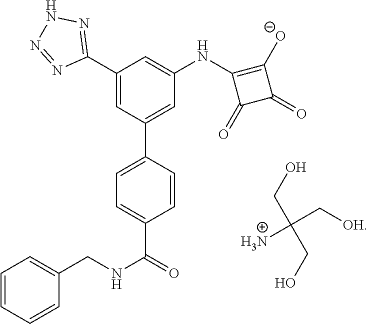 [SMILES: O=C(NCc1ccccc1)c1ccc(-c2cc(Nc3c([O-])c(=O)c3=O)cc(-c3nn[nH]n3)c2)cc1 ; [NH3+]C(CO)(CO)CO]

19. (canceled)

20. A pharmaceutical composition comprising the compound or salt according to claim 1 and a pharmaceutically acceptable excipient.

21. (canceled)

22. A method of treating gastrointestinal disorders or conditions, symptoms of pain associated with gastrointestinal disease or other visceral conditions, or pulmonary diseases or conditions in a subject in need thereof, comprising administering to the subject a therapeutically effective amount of a compound or salt according to claim 1.

23. The method according to claim 22, wherein the gastrointestinal disorder or condition is selected from the group consisting of food allergy, food intolerance and allergic disorders, celiac disease, gastrointestinal symptoms associated with systemic mastocytosis and other mast cell related disorders (mast cell activation syndrome, clonal mast cell disorder, monoclonal mast cell activation syndrome, idiopathic urticaria, idiopathic anaphylaxis), mastocytic colitis, irritable bowel syndrome (IBS), gastrointestinal motility disorders, functional gastrointestinal disorders, gastroesophageal reflux disease (GERD), duodenogastric reflux, diarrhoeal diseases, eosinophilic gastroenteritis, eosinophilic esophagitis, infectious diarrhea (such as Clostridium difficile, Salmonella, Shigella toxin), microscopic colitis, immune mediated gastrointestinal diseases, Crohn's disease, ulcerative colitis, inflammatory bowel disease, and visceral abdominal pain.

24. The method according to claim 22, wherein the symptoms of pain are associated with a gastrointestinal disease or other visceral condition selected from the group consisting of: Crohn's disease, ulcerative colitis, inflammatory bowel disease, radiation colitis, radiation cystitis, celiac disease, gluten enteropathy, radiation cystitis, interstitial cystitis, painful bladder syndrome, cancer, gastroesophageal reflux disease, chemotherapy and radiotherapy mucositis, pancreatitis, prostatitis, pelvic pain, endometriosis, hepatitis, hepatic fibrosis, and cirrhosis.

25. The method according to claim 22, wherein the pulmonary disease or condition is selected from the group consisting of: chronic obstructive pulmonary diseases, asthma, chronic bronchitis, cystic fibrosis, emphysema, chronic idiopathic cough, hyperactive airway disorder, and idiopathic pulmonary fibrosis.

26. (canceled)

27. The method according to claim 22, wherein the subject is a human.

28. The pharmaceutically acceptable salt according to claim 18, wherein the salt is in a crystalline form.

29. The crystalline form according to claim 28, which is a hydrate.

30. The crystalline form according to claim 29, which has an XRPD pattern substantially in accordance with FIG. 1.

31. The crystalline form according to claim 29, which has an XRPD pattern comprising diffraction angles at 3.9±0.2, 7.7±0.2, 10.0±0.2, and 15.8±0.2°2θ, when measured using Cu K_{α} radiation.

32. The crystalline form according to claim 29, which has an XRPD pattern substantially in accordance with FIG. 4.

33. The crystalline form according to claim 29, which has an XRPD pattern comprising diffraction angles at 4.4±0.2, 14.4±0.2, and 23.9±0.2 020, when measured using Cu K_{α} radiation.
